SEQUENCE LISTING (33 sequences)

SEQ ID NO: 1 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 4 - protein, 441 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 219 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 215 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 450 aa, synthetic construct; identical to SEQ ID NO: 7

SEQ ID NO: 9 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, synthetic construct; identical to SEQ ID NO: 9

SEQ ID NO: 11 - protein, 449 aa, synthetic construct
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLTVSSASTKGPSVFPLAPCSRSTSESTAALGCLVKDYFPEPVTVSWNSGALTSGVHTFPALQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 450 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 13 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 445 aa, synthetic construct
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 451 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTSKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 16 - protein, 214 aa, synthetic construct
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 21 - protein, 215 aa, synthetic construct; identical to SEQ ID NO: 17

SEQ ID NO: 22 - protein, 391 aa, Homo sapiens
MAEESSCTRDCMSFSVLNWDQVSRLHEVLTEVVPIHGRGNFPTLEITLKDIVQTVRSRLEEAGIKVHDVRLNGSAAGHVLVKDNGLGCKDLDLIFHVALPTEAEFQLVRDVVLCSLLNFLPEGVNKLKISPVTLKEAYVQKLVKVCTDTDRWSLISLSNKNGKNVELKFVDSIRRQFEFSVDSFQIILDSLLFFYDCSNNPISEHFHPTVIGESMYGDFEEAFDHLQNRLIATKNPEEIRGGGLLKYSNLLVRDFRPTDQEEIKTLERYMCSRFFIDFPDILEQQRKLETYLQNHFAEEERSKYDYLMILRRVVNESTVCLMGHERRQTLNLISLLALRVLAEQNIIPSATNVTCYYQPAPYVSDGNFSNYYVAHPPVTYSQPYPTWLPCN

SEQ ID NO: 23 - DNA, 1176 bp, Homo sapiens
  source 1..1176: mol_type = genomic DNA
atggcagaggagagcagctgtaccagggattgcatgtccttcagcgtgctcaactgggatcaggttagccggctgcatgaggtcctcactgaagttgtacctatccacggacgaggcaactttccaaccttggagataactctgaaggacatcgtccagaccgtccgcagtcggctggaggaggcaggcatcaaagtgcacgacgtccggctgaatggctccgcagctggccacgttttggtcaaagacaatggcttgggctgcaaagacctggacctaatcttccatgtggctcttccaacagaggcagaatttcagctggttagagatgtggttctgtgttcccttctgaacttcctgccagagggtgtgaacaagctcaaaatcagtccagtcactctgaaggaggcatatgtgcagaagctagtgaaggtttgcacggacactgaccgctggagcctgatctccctctccaacaagaacgggaagaacgtggagctgaagtttgtcgactccattcggcgtcagtttgagttcagtgtggactctttccaaatcatcctggattctttgcttttcttctatgactgttccaataatcccatctctgagcacttccaccccaccgtgattggggagagcatgtacggggactttgaggaagcttttgaccatctgcagaacagactgatcgccaccaagaacccagaagaaatcagaggcgggggacttctcaagtacagcaaccttcttgtgcgggacttcaggcccacagaccaggaagaaatcaaaactctagagcgctacatgtgctccaggttcttcatcgacttcccggacatccttgaacagcagaggaagttggagacttaccttcaaaaccacttcgctgaagaagagagaagcaagtacgactacctcatgatccttcgcagggtggtgaacgagagcaccgtgtgtctcatggggcatgaacgcaggcagactctgaacctcatctccctcctggccttgcgtgtgctggcggaacaaaacatcatccccagtgccaccaacgtcacctgttactaccagccggccccttacgtcagtgatggcaacttcagcaactactacgttgcccatcctccagtcacctacagccagccttaccctacctggctgccctgtaactaa

SEQ ID NO: 24 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 25 - DNA, 567 bp, Homo sapiens
  source 1..567: mol_type = genomic DNA
atgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 26 - protein, 383 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFMYPRESSTSVM

SEQ ID NO: 27 - DNA, 1152 bp, Homo sapiens
  source 1..1152: mol_type = genomic DNA
atggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcatgtacccaagggaatcctctacatcagtgatgtga

SEQ ID NO: 28 - protein, 323 aa, Homo sapiens
MAAELSMGPELPTSPLAMEYVNDFDLLKFDVKKEPLGRAERPGRPCTRLQPVGSVSSTPLSTPCSSVPSSPSFSPTEQKTHLEDLYWMASNYQQMNPEALNLTPEDAVEALIGSHPVPQPLQSFDSFRGAHHHHHHHHPHPHHAYPGAGVAHDELGPHAHPHHHHHHQASPPPSSAASPAQQLPTSHPGPGPHATASATAAGGNGSVEDRFSDDQLVSMSVRELNRHLRGFTKDEVIRLKHKRRTLKNRGYAQSCRYKRVQQKHHLENEKTQLIQQVEQLKQEVSRLARERDAYKVKCEKLANSGFREAGSTSDSPSSPEFFL

SEQ ID NO: 29 - DNA, 972 bp, Homo sapiens
  source 1..972: mol_type = genomic DNA
atggccgcggagctgagcatggggccagagctgcccaccagcccgctggccatggagtatgtcaacgacttcgacctgctcaagttcgacgtgaagaaggagccactggggcgcgcggagcgtccgggcaggccctgcacacgcctgcagccagtcggctcggtgtcctccacaccgctcagcactccgtgtagctccgtgccctcgtcgcccagcttcagcccgaccgaacagaagacacacctcgaggatctgtactggatggcgagcaactaccagcagatgaaccccgaggcgctcaacctgacgcccgaggacgcggtggaagcgctcatcggctcgcacccagtgccacagccgctgcaaagcttcgacagctttcgcggcgctcaccaccaccaccatcaccaccaccctcacccgcaccacgcgtacccgggcgccggcgtggcccacgacgagctgggcccgcacgctcacccgcaccatcaccatcatcaccaagcgtcgccgccgccgtccagcgccgctagcccggcgcaacagctgcccactagccaccccgggcccgggccgcacgcgacggcctcggcgacggcggcgggcggcaacggcagcgtggaggaccgcttctccgacgaccagctcgtgtccatgtccgtgcgcgagctgaaccgccacctgcggggcttcaccaaggacgaggtgatccgcctgaagcacaagcggcggaccctgaagaaccggggctacgcccagtcttgcaggtataaacgcgtccagcagaagcaccacctggagaatgagaagacgcagctcattcagcaggtggagcagcttaagcaggaggtgtcccggctggcccgcgagagagacgcctacaaggtcaagtgcgagaaactcgccaactccggcttcagggaggcgggctccaccagcgacagcccctcctctcccgagttctttctgtga

SEQ ID NO: 30 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 31 - DNA, 1320 bp, Homo sapiens
  source 1..1320: mol_type = genomic DNA
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 32 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 33 - DNA, 570 bp, Homo sapiens
  source 1..570: mol_type = genomic DNA
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

(19) United States
(12) Patent Application Publication
(10) Pub. No.: US 2026/0347948 A1
(43) Pub. Date: Nov. 5, 2026

(54) METHODS FOR TREATMENT AND ASSESSMENT OF MULTIPLE MYELOMA OR PRECURSORS THEREOF

(71) Applicants: DANA-FARBER CANCER INSTITUTE, INC., Boston, MA (US); THE GENERAL HOSPITAL CORPORATION, Boston, MA (US)

(72) Inventors: Gad Getz, Belmont, MA (US); Irene Ghobrial, Boston, MA (US); Jean-Baptiste Alberge, Vanves (FR); Ankit Kumar Dutta, Boston, MA (US)

(21) Appl. No.: 19/697,129

(22) Filed: Jun. 3, 2026

Related U.S. Application Data
(63) Continuation of application No. PCT/US2025/028186, filed on May 7, 2025.
(60) Provisional application No. 63/643,755, filed on May 7, 2024.

(51) Int. Cl. C12Q 1/6886

(52) U.S. Cl. CPC C12Q 1/6886; C12Q 2600/118; C12Q 2600/156

(57) ABSTRACT

Methods for treating and/or assessing a subject having a symptomatic multiple myeloma or a precursor thereof, where the methods involve characterizing a plasma cell dyscrasia (e.g., monoclonal gammopathy of undetermined significance, smoldering multiple myeloma, multiple myeloma, plasma cell leukemia) in a biological sample from a subject as being high, medium, or low risk by assigning to the plasma cell dyscrasia a multiple myeloma (MM)-like score. The methods may involve sequencing DNA from the biological sample using whole-genome sequencing (WGS).

### CROSS-REFERENCE TO RELATED APPLICATIONS

This application claims the benefit of U.S. Provisional Application Ser. No. 63/643,755, filed on May 7, 2024, the disclosure of which is incorporated by reference herein in its entirety.

### STATEMENT OF RIGHTS TO INVENTIONS MADE UNDER FEDERALLY SPONSORED RESEARCH

The claimed invention was made with government support under CA263817 awarded by the National Institutes of Health. The government has certain rights in the claimed invention.

### SEQUENCE LISTING

This application contains a Sequence Listing electronically submitted via EFS-Web to the United States Patent and Trademark Office as an XML file entitled “0680.003473WO01.xml” having a size of 44,085 bytes and created on May 1, 2025. The information contained in the Sequence Listing is incorporated by reference herein.

### BACKGROUND

Precursor stages of multiple myeloma (MM), including monoclonal gammopathy of undetermined significance (MGUS) and smoldering multiple myeloma (SMM), are heterogeneous diseases with overall rates of progression to overt MM of 1% to 10% per year. Currently, MGUS and SMM patients are closely observed in the clinic for signs of progression, using surrogate clinical variables that reflect tumor biology, such as levels of M-spike and free light chain, as well as plasma cell infiltration in the bone marrow (BM). Established stratification models use these biomarkers to prognosticate patients and determine their risk category; however, different models can yield discordant results. Furthermore, the commonly used stratification systems do not include somatic alterations that play a role in the disease. With the democratization of next-generation sequencing technologies, profiling of genetic biomarkers based on clinical whole genome sequencing (WGS) offers a robust solution for precision oncology with informed risk assessment, stratification, and prediction of progression. Being able to molecularly discriminate which premalignant patients are at risk and on a trajectory for the disease to progress will address a major unmet clinical need and allow for improved treatment decisions with the goal of intercepting transformation and preventing the irreversible organ damage that is manifested in MM.

### SUMMARY

As described below, the present disclosure features methods for treating a subject having a symptomatic multiple myeloma or a precursor thereof, where the methods involve characterizing a plasma cell dyscrasia (e.g., monoclonal gammopathy of undetermined significance, smoldering multiple myeloma, multiple myeloma, plasma cell leukemia) in a biological sample from a subject as being high, medium, or low risk by assigning to the plasma cell dyscrasia a multiple myeloma (MM)-like score. In some cases, the methods involve sequencing DNA from the biological sample using whole-genome sequencing (WGS).

In one aspect, the disclosure features a method for selecting a subject having monoclonal gammopathy of undetermined significance (MGUS) or smoldering multiple myeloma (SMM) for administration of a therapeutic agent. The method involves A) determining whether or not a biological sample from the subject contains a marker listed for each of the following categories a) to n): a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2). The method also involves B) calculating an MM-like score for the subject, where the MM-like score is calculated by summing values assigned to each of categories a) to n), where each category is assigned a value of 0 if no markers in the category are detected, where each of categories a) and b) are assigned a value of −1 if a marker within the category is detected, and where each of categories c) to n) are assigned a value of +1 if a marker within the category is detected. The method further involves C) selecting the subject for administration of the therapeutic agent if the MM-like score is equal to or greater than 4. The subject is not selected for administration of the therapeutic agent if the MM-like score is less than 4.

In another aspect, the disclosure features a method for selecting a subject diagnosed as having multiple myeloma (MM) for administration of a therapeutic agent or autologous stem cell transplant for treating MM. The method involves A) determining whether or not a biological sample from the subject contains a marker listed for each of the following categories a) to n): a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2). The method also involves B) calculating an MM-like score for the subject, where the MM-like score is calculated by summing values assigned to each of categories a) to n), where each category is assigned a value of 0 if no markers in the category are detected, where each of categories a) and b) are assigned a value of −1 if a marker within the category is detected, and where each of categories c) to n) are assigned a value of +1 if a marker within the category is detected. The method further involves C) selecting the subject for administration of the therapeutic agent or autologous stem cell transplant if the MM-like score is equal to or greater than 2 and the subject is eligible for an autologous stem cell transplant, or selecting the subject for administration of the therapeutic agent if the subject is not eligible for an autologous stem cell transplant. The subject is not selected for administration of the therapeutic agent or autologous stem cell transplant if the MM-like score is less than 2.

In another aspect, the disclosure features a method for selecting a subject having monoclonal gammopathy of undetermined significance (MGUS), smoldering multiple myeloma (SMM) or multiple myeloma (MM) for inclusion in or exclusion from a clinical trial to study an agent for treatment of MM. The method involves A) determining whether or not a biological sample from the subject contains a marker listed for each of the following categories a) to n): a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2); B) calculating an MM-like score for the subject, where the MM-like score is calculated by summing values assigned to each of categories a) to n), where each category is assigned a value of 0 if no markers in the category are detected, where each of categories a) and b) are assigned a value of −1 if a marker within the category is detected, and where each of categories c) to n) are assigned a value of +1 if a marker within the category is detected. The method further involves C) selecting the subject for inclusion in the clinical trial if the subject has MGUS or SMM and the MM-like score is greater than or equal to 4, and excluding the subject form the clinical trial if the subject has MGUS or SMM and the MM-like score is less than 4.

In any aspect of the disclosure, or embodiments thereof, the subject is selected for administration of the therapeutic agent if the MM-like score is greater than 5. In any aspect of the disclosure, or embodiments thereof, the subject is selected for administration of the therapeutic agent if the MM-like score is greater than 6. In any aspect of the disclosure, or embodiments thereof, the MM-like score is equal to 2 or 3. In any aspect of the disclosure, or embodiments thereof, the MM-like score is greater than or equal to 4. In any aspect of the disclosure, or embodiments thereof, the MM-like score is less than or equal to 3. In any aspect of the disclosure, or embodiments thereof, the MM-like score is greater than 4.

In any aspect of the disclosure, or embodiments thereof, the biological sample is a blood sample. In any aspect of the disclosure, or embodiments thereof, the biological sample is a bone marrow sample.

In any aspect of the disclosure, or embodiments thereof, determining whether or not the biological sample from the subject contains a marker listed for each of categories a) to n) involves analyzing sequencing data to identify the markers. The sequencing data is produced by sequencing polynucleotides from the biological sample using whole-genome sequencing. In any aspect of the disclosure, or embodiments thereof, the whole-genome sequencing involves sequencing the polynucleotides to a coverage of at least 10-fold. In any aspect of the disclosure, or embodiments thereof, the whole-genome sequencing involves sequencing the polynucleotides to a coverage of at least 100-fold. In any aspect of the disclosure, or embodiments thereof, sequencing polynucleotides from the biological sample using whole-genome sequencing involves enriching cancer cells from the biological sample and sequencing polynucleotides from the enriched cancer cells.

In any aspect of the disclosure, or embodiments thereof, the polynucleotides contain DNA. In any aspect of the disclosure, or embodiments thereof, the polynucleotides contain RNA.

In any aspect of the disclosure, or embodiments thereof, the therapeutic agent contains a BCMAxCD3 bispecific antibody. In any aspect of the disclosure, or embodiments thereof, the BCMAxCD3 bispecific antibody is selected from one or more of teclistamab, elranatamab, and linvoseltamab. In any aspect of the disclosure, or embodiments thereof, the therapeutic agent contains a GPRC5DxCD3 bispecific antibody. In any aspect of the disclosure, or embodiments thereof, the GPRC5DxCD3 bispecific antibody is talquetamab. In any aspect of the disclosure, or embodiments thereof, the therapeutic agent contains a BCMA-directed CAR-T cell. In any aspect of the disclosure, or embodiments thereof, the BCMA-directed CAR-T cell is ciltacabtagene autoleucel or idecabtagene vicleucel. In any aspect of the disclosure, or embodiments thereof, the therapeutic agent contains an immunomodulatory agent and a glucocorticoid. In any aspect of the disclosure, or embodiments thereof, the immunomodulatory agent is lenalidomide and the glucocorticoid is dexamethasone. In any aspect of the disclosure, or embodiments thereof, the therapeutic agent contains an anti-CD38 monoclonal antibody, an immunomodulatory agent, and a glucocorticoid. In any aspect of the disclosure, or embodiments thereof, the anti-CD38 monoclonal antibody is daratumumab or isatuximab, the immunomodulatory agent is lenalidomide, and the glucocorticoid is dexamethasone.

In any aspect of the disclosure, or embodiments thereof, the subject is eligible for an autologous stem cell transplant. In any aspect of the disclosure, or embodiments thereof, the therapeutic agent is a chemotherapeutic agent.

In any aspect of the disclosure, or embodiments thereof, the therapeutic agent is a combination of the following: a) bortezomib, b) lenalidomide, thalidomide, or cyclophosphamide, and c) dexamethasone. In any aspect of the disclosure, or embodiments thereof, the method further involves administering to the subject lenalinomide as a maintenance treatment.

In any aspect of the disclosure, or embodiments thereof, the method involves administering the autologous stem cell transplant to the subject.

In any aspect of the disclosure, or embodiments thereof, the therapeutic agent is a combination of the following: a) daratumumab, b) lenalidomide, iberdomide, or mezigdomide, d) bortezomib or carfilzomib, and d) dexamethasone.

In any aspect of the disclosure, or embodiments thereof, the method further involves determining whether or not the biological sample contains the marker t(11;14), where if the biological sample contains the marker t(11;14), the therapeutic agent contains venetoclax.

In any aspect of the disclosure, or embodiments thereof, the subject is not eligible for an autologous stem cell transplant.

In any aspect of the disclosure, or embodiments thereof, the therapeutic agent is a combination of the following: a) bortezomib, b) lenalidomide, and c) dexamethasone. In any aspect of the disclosure, or embodiments thereof, the therapeutic agent is a combination of the following: a) daratumumab, b) lenalidomide, and c) dexamethasone.

In any aspect of the disclosure, or embodiments thereof, the method further involves administering to the subject lenalinomide as a maintenance treatment.

In any aspect of the disclosure, or embodiments thereof, the method further involves administering to the subject lenalinomide and/or bortezomib as a maintenance treatment.

Compositions and articles defined by the disclosure were isolated or otherwise manufactured in connection with the examples provided below. Other features and advantages of the embodiments of the disclosure will be apparent from the detailed description, and from the claims.

### DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change in the structure, expression levels or activity of a polynucleotide or polypeptide as detected by standard art known methods such as those described herein. The alteration can be an increase or a decrease. As used herein, an alteration includes a 10% change in expression levels, a 25% change, a 40% change, and a 50% or greater change in expression levels.

The term “amplification” means any method employing a primer and a polymerase capable of replicating a target sequence with reasonable fidelity. In embodiments the target sequence is a genome, and the amplification is “whole-genome amplification.” In some instances, a method of the disclosure may comprise an amplification step (e.g., a PCR step for adapter ligation and/or final library amplification) but specifically exclude any whole-genome amplification step, which is typically a preceding step when sequencing samples contain low levels of DNA (e.g., low numbers of cells). In some embodiments, the methods of the disclosure do not include any amplification of a DNA sample (e.g., whole genome amplification) prior to library preparation. Amplification may be carried out by natural or recombinant DNA polymerases such as TAQGOLD, T7 DNA polymerase, Klenow fragment of E. coli DNA polymerase, and reverse transcriptase. Non-limiting examples of amplification methods include PCR and/or whole genome amplification. Amplification may involve thermocycling or isothermal amplification (such as through the methods RPA or LAMP).

By “analog” is meant a molecule that is not identical but has analogous functional or structural features. For example, a polypeptide analog retains the biological activity of a corresponding naturally occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “aneuploidy” is meant in the context of a cell having an abnormal number of chromosomes relative to a cell of normal ploidy.

As used herein, the term “antibody” or “antigen-binding domain” refers to an immunoglobulin molecule or a fragment thereof that specifically binds to, or is immunologically reactive with, a particular antigen. In some embodiments, an antibody is a “bispecific antibody,” which is an immunoglobulin molecule, polypeptide, polypeptide complex, or composition containing two antibodies that binds to, or is immunologically reactive with, two antigens. Non-limiting examples of antibodies or antigen-binding domains include polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments, as well as engineered antibodies, which include CrossMabs (e.g., CrossMab^{Fabs}, CrossMab^{CH1-CL} and CrossMab^{VH-VL} formats), or fragments thereof. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983).

Representative heavy chain and light chain sequences for antibodies of the disclosure are provided in Table A below. In various embodiments, the listed antibody contains a sequence having at least about 85% sequence identity to one or more of the listed heavy chains and/or light chains.

TABLE A
Representative antibody sequences.

| Antibody | | |
|---|---|---|
| Name | Heavy Chain(s) | Light Chain(s) |
| daratumumab | >Heavy Chain | >Light Chain |
| | EVQLLESGGGLVQPGGSLRLSCAVS | EIVLTQSPATLSLSPGERATLSCR |
| | GFTENS FAMSWVRQAPGKGLEWVSA | ASQSVSSYLAWYQQKPGQAPRLLI |
| | ISGSGGGTYYADSVKGRFTISRDNS | YDASNRATGIPARFSGSGSGTDFT |
| | KNTLYLQMNSLRAEDTAVYFCAKDK | LTISSLEPEDFAVYYCQQRSNWPP |
| | ILWFGEPVFDYWGQGTLVTVSSAST | TFGQGTKVEIKRTVAAPSVFIFPP |
| | KGPSVFPLAPSSKSTSGGTAALGCL | SDEQLKSGTASVVCLLNNFYPREA |
| | VKDYFPEPVTVSWNSGALTSGVHTF | KVQWKVDNALQSGNSQESVTEQDS |
| | PAVLQSSGLYSLSSVVTVPSSSLGT | KDSTYSLSSTLTLSKADYEKHKVY |
| | QTYICNVNHKPSNTKVDKRVEPKSC | ACEVTHQGLSSPVTKSENRGEC |
| | DKTHTCPPCPAPELLGGPSVFLFPP | (SEQ ID NO: 2) |
| | KPKDTLMISRTPEVTCVVVDVSHED | |
| | PEVKENWYVDGVEVHNAKTKPREEQ | |
| | YNSTYRVVSVLTVLHQDWLNGKEYK | |
| | CKVSNKALPAPIEKTISKAKGQPRE | |
| | PQVYTLPPSREEMTKNQVSLTCLVK | |
| | GFYPSDIAVEWESNGQPENNYKTTP | |
| | PVLDSDGSFFLYSKLTVDKSRWQQG | |
| | NVFSCSVMHEALHNHYTQKSLSLSP | |
| | GK (SEQ ID NO: 1) | |
| elranatamab | >Heavy chain 1 | >Light chain 1 |
| | EVQLVESGGGLVQPGGSLRLSCAAS | DIVMTQSPDSLAVSLGERATINCK |
| | GFTFSDYYMTWVRQAPGKGLEWVAF | SSQSLFNVRSRKNYLAWYQQKPGQ |
| | IRNRARGYTSDHNPSVKGRFTISRD | PPKLLISWASTRESGVPDRESGSG |
| | NAKNSLYLQMNSLRAEDTAVYYCAR | SGTDFTLTISSLQAEDVAVYYCKQ |
| | DRPSYYVLDYWGQGTTVTVSSASTK | SYDLFTFGSGTKLEIKRTVAAPSV |
| | GPSVFPLAPCSRSTSESTAALGCLV | FIFPPSDEQLKSGTASVVCLLNNF |
| | KDYFPEPVTVSWNSGALTSGVHTFP | YPREAKVQWKVDNALQSGNSQESV |
| | AVLQSSGLYSLSSVVTVPSSNFGTQ | TEQDSKDSTYSLSSTLTLSKADYE |
| | TYTCNVDHKPSNTKVDKTVERKCRV | KHKVYACEVTHQGLSSPVTKSENR |
| | RCPRCPAPPVAGPSVFLFPPKPKDT | GEC (SEQ ID NO: 5) |
| | LMISRTPEVTCVVVAVSHEDPEVQF | >Light chain 2 |
| | NWYVDGVEVHNAKTKPREEQFNSTF | EIVLTQSPGTLSLSPGERATLSCR |
| | RVVSVLTVVHQDWLNGKEYKCKVSN | ASQSVSSSYLAWYQQKPGQAPRLL |
| | KGLPSSIEKTI SKTKGQPREPQVYT | MYDASIRATGIPDRESGSGSGTDE |
| | LPPSREEMTKNQVSLTCLVKGFYPS | TLTISRLEPEDFAVYYCQQYQSWP |
| | DIAVEWESNGQPENNYKTTPPMLDS | LTFGQGTKVEIKRTVAAPSVFIFP |
| | DGSFFLYSRLTVDKSRWQQGNVFSC | PSDEQLKSGTASVVCLLNNFYPRE |
| | SVMHEALHNHYTQKSLSLSPGK | AKVQWKVDNALQSGNSQESVTEQD |
| | (SEQ ID NO: 3) | SKDSTYSLSSTLTLSKADYEKHKV |
| | >Heavy Chain 2 | YACEVTHQGLSSPVTKSENRGEC |
| | EVQLLESGGGLVQPGGSLRLSCAAS | (SEQ ID NO: 6) |
| | GFTFSSYPMSWVRQAPGKGLEWVSA | |
| | IGGSGGSLPYADIVKGRFTISRDNS | |
| | KNTLYLQMNSLRAEDTAVYYCARYW | |
| | PMDIWGQGTLVTVSSASTKGPSVFP | |
| | LAPCSRSTSESTAALGCLVKDYFPE | |
| | PVTVSWNSGALTSGVHTFPAVLQSS | |
| | GLYSLSSVVTVPSSNFGTQTYTCNV | |
| | DHKPSNTKVDKTVERKCEVECPECP | |
| | APPVAGPSVFLFPPKPKDTLMISRT | |
| | PEVTCVVVAVSHEDPEVQFNWYVDG | |
| | VEVHNAKTKPREEQFNSTFRVVSVL | |
| | TVVHQDWLNGKEYKCKVSNKGLPSS | |
| | IEKTISKTKGQPREPQVYTLPPSRE | |
| | EMTKNQVSLTCEVKGFYPSDIAVEW | |
| | ESNGQPENNYKTTPPMLDSDGSFFL | |
| | YSKLTVDKSRWQQGNVFSCSVMHEA | |
| | LHNHYTQKSLSLSPGK (SEQ ID | |
| | NO: 4) | |
| isatuximab | >A chain | >C chain |
| | QVQLVQSGAEVAKPGTSVKLSCKAS | DIVMTQSHLSMSTSLGDPVSITCK |
| | GYTFTDYWMQWVKQRPGQGLEWIGT | ASQDVSTVVAWYQQKPGQSPRRLI |
| | IYPGDGDTGYAQKFQGKATLTADKS | YSASYRYIGVPDRFTGSGAGTDFT |
| | SKTVYMHLSSLASEDSAVYYCARGD | FTISSVQAEDLAVYYCQQHYSPPY |
| | YYGSNSLDYWGQGTSVTVSSASTKG | TFGGGTKLEIKRTVAAPSVFIFPP |
| | PSVFPLAPSSKSTSGGTAALGCLVK | SDEQLKSGTASVVCLLNNFYPREA |
| | DYFPEPVTVSWNSGALTSGVHTFPA | KVQWKVDNALQSGNSQESVTEQDS |
| | VLQSSGLYSLSSVVTVPSSSLGTQT | KDSTYSLSSTLTLSKADYEKHKVY |
| | YICNVNHKPSNTKVDKKVEPKSCDK | ACEVTHQGLSSPVTKSENRGEC |
| | THTCPPCPAPELLGGPSVFLFPPKP | (SEQ ID NO: 9) |
| | KDTLMISRTPEVTCVVVDVSHEDPE | >D chain |
| | VKFNWYVDGVEVHNAKTKPREEQYN | DIVMTQSHLSMSTSLGDPVSITCK |
| | STYRVVSVLTVLHQDWLNGKEYKCK | ASQDVSTVVAWYQQKPGQSPRRLI |
| | VSNKALPAPIEKTISKAKGQPREPQ | YSASYRYIGVPDRFTGSGAGTDFT |
| | VYTLPPSRDELTKNQVSLTCLVKGF | FTISSVQAEDLAVYYCQQHYSPPY |
| | YPSDIAVEWESNGQPENNYKTTPPV | TFGGGTKLEIKRTVAAPSVFIFPP |
| | LDSDGSFFLYSKLTVDKSRWQQGNV | SDEQLKSGTASVVCLLNNFYPREA |
| | FSCSVMHEALHNHYTQKSLSLSPGK | KVQWKVDNALQSGNSQESVTEQDS |
| | (SEQ ID NO: 7) | KDSTYSLSSTLTLSKADYEKHKVY |
| | >B chain | ACEVTHQGLSSPVTKSENRGEC |
| | QVQLVQSGAEVAKPGTSVKLSCKAS | (SEQ ID NO: 10) |
| | GYTFTDYWMQWVKQRPGQGLEWIGT | |
| | IYPGDGDTGYAQKFQGKATLTADKS | |
| | SKTVYMHLSSLASEDSAVYYCARGD | |
| | YYGSNSLDYWGQGTSVTVSSASTKG | |
| | PSVFPLAPSSKSTSGGTAALGCLVK | |
| | DYFPEPVTVSWNSGALTSGVHTFPA | |
| | VLQSSGLYSLSSVVTVPSSSLGTQT | |
| | YICNVNHKPSNTKVDKKVEPKSCDK | |
| | THTCPPCPAPELLGGPSVFLFPPKP | |
| | KDTLMISRTPEVTCVVVDVSHEDPE | |
| | VKFNWYVDGVEVHNAKTKPREEQYN | |
| | STYRVVSVLTVLHQDWLNGKEYKCK | |
| | VSNKALPAPIEKTISKAKGQPREPQ | |
| | VYTLPPSRDELTKNQVSLTCLVKGF | |
| | YPSDIAVEWESNGQPENNYKTTPPV | |
| | LDSDGSFFLYSKLTVDKSRWQQGNV | |
| | FSCSVMHEALHNHYTQKSLSLSPGK | |
| | (SEQ ID NO: 8) | |
| linvoseltamab | >Heavy chain 1 | >Light chain |
| | EVQLVESGGGLVQPGGSLRLSCAAS | DIQMTQSPSSLSASVGDRVTITCR |
| | GFTFSNFWMTWVRQAPGKGLEWVAN | ASQSISSYLNWYQQKPGKAPKLLI |
| | MNQDGSEKYYVDSVKGRFTISRDNA | YAASSLQSGVPSRFSGSGSGTDFT |
| | KSSLYLQMNSLRAEDTAVYYCARDR | LTISSLQPEDFATYYCQQSYSTPP |
| | EYCISTSCYDDFDYWGQGTLTVSSA | ITFGQGTRLEIKRTVAAPSVFIFP |
| | STKGPSVFPLAPCSRSTSESTAALG | PSDEQLKSGTASVVCLLNNFYPRE |
| | CLVKDYFPEPVTVSWNSGALTSGVH | AKVQWKVDNALQSGNSQESVTEQD |
| | TFPALQSSGLYSLSSVVTVPSSSLG | SKDSTYSLSSTLTLSKADYEKHKV |
| | TKTYTCNVDHKPSNTKVDKRVESKY | YACEVTHQGLSSPVTKSENRGEC |
| | GPPCPPCPAPPVAGPSVFLFPPKPK | (SEQ ID NO: 13) |
| | DTLMISRTPEVTCVVVDVSQEDPEV | |
| | QFNWYVDGVEVHNAKTKPREEQENS | |
| | TYRVVSVLTVLHQDWLNGKEYKCKV | |
| | SNKGLPSSIEKTISKAKGQPREPQV | |
| | YTLPPSQEEMTKNQVSLTCLVKGFY | |
| | PSDIAVEWESNGQPENNYKTTPPVL | |
| | DSDGSFFLYSRLTVDKSRWQEGNVF | |
| | SCSVMHEALHNHYTQKSLSLSLGK | |
| | (SEQ ID NO: 11) | |
| | >Heavy chain 2 | |
| | EVQLVESGGGLVQPGRSLRLSCAAS | |
| | GFTEDDYSMHWVRQAPGKGLEWVSG | |
| | ISWNSGSKGYADSVKGRFTISRDNA | |
| | KNSLYLQMNSLRAEDTALYYCAKYG | |
| | SGYGKFYHYGLDVWGQGTTVTVSSA | |
| | STKGPSVFPLAPCSRSTSESTAALG | |
| | CLVKDYFPEPVTVSWNSGALTSGVH | |
| | TFPAVLQSSGLYSLSSVVTVPSSSL | |
| | GTKTYTCNVDHKPSNTKVDKRVESK | |
| | YGPPCPPCPAPPVAGPSVFLFPPKP | |
| | KDTLMISRTPEVTCVVVDVSQEDPE | |
| | VQFNWYVDGVEVHNAKTKPREEQFN | |
| | STYRVVSVLTVLHQDWLNGKEYKCK | |
| | VSNKGLPSSIEKTISKAKGQPREPQ | |
| | VYTLPPSQEEMTKNQVSLTCLVKGF | |
| | YPSDIAVEWESNGQPENNYKTTPPV | |
| | LDSDGSFFLYSRLTVDKSRWQEGNV | |
| | FSCSVMHEALHNRFTQKSLSLSPGK | |
| | (SEQ ID NO: 12) | |
| talquetamab | >Heavy chain 1 | >Light chain 1 |
| | QVQLVQSGAEVKKPGASVKVSCKAS | DIQMTQSPSSLSASVGDRVTITCK |
| | GYSFTGYTMNWVRQAPGQGLEWMGL | ASQNVATHVGWYQQKPGKAPKRLI |
| | INPYNSDTNYAQKLQGRVTMTTDTS | YSASYRYSGVPSRFSGSGSGTEFT |
| | TSTAYMELRSLRSDDTAVYYCARVA | LTISNLQPEDFATYYCQQYNRYPY |
| | LRVALDYWGQGTLVTVSSASTKGPS | TFGQGTKLEIKRTVAAPSVFIFPP |
| | VFPLAPCSRSTSESTAALGCLVKDY | SDEQLKSGTASVVCLLNNFYPREA |
| | FPEPVTVSWNSGALTSGVHTFPAVL | KVQWKVDNALQSGNSQESVTEQDS |
| | QSSGLYSLSSVVTVPSSSLGTKTYT | KDSTYSLSSTLTLSKADYEKHKVY |
| | CNVDHKPSNTKVDKRVESKYGPPCP | ACEVTHQGLSSPVTKSENRGEC |
| | PCPAPEAAGGPSVFLFPPKPKDTLM | (SEQ ID NO: 16) |
| | ISRTPEVTCVVVDVSQEDPEVQFNW | >Light chain 2 |
| | YVDGVEVHNAKTKPREEQFNSTYRV | QTVVTQEPSLTVSPGGTVTLTCRS |
| | VSVLTVLHQDWLNGKEYKCKVSNKG | STGAVTTSNYANWVQQKPGQAPRG |
| | LPSSIEKTISKAKGQPREPQVYTLP | LIGGTNKRAPGTPARFSGSLLGGK |
| | PSQEEMTKNQVSLTCLVKGFYPSDI | AALTLSGVQPEDEAEYYCALWYSN |
| | AVEWESNGQPENNYKTTPPVLDSDG | LWVFGGGTKLTVLGQPKAAPSVTL |
| | SFFLYSRLTVDKSRWQEGNVFSCSV | FPPSSEELQANKATLVCLISDFYP |
| | MHEALHNHYTQKSLSLSLGK (SEQ | GAVTVAWKADSSPVKAGVETTTPS |
| | ID NO: 14) | KQSNNKYAASSYLSLTPEQWKSHR |
| | >Heavy chain 2 | SYSCQVTHEGSTVEKTVAPTECS |
| | EVQLVESGGGLVQPGGSLRLSCAAS | (SEQ ID NO: 17) |
| | GFTENTYAMNWVRQAPGKGLEWVAR | |
| | IRSKYNNYATYYAASVKGRFTISRD | |
| | DSKNSLYLQMNSLKTEDTAVYYCAR | |
| | HGNFGNSYVSWFAYWGQGTLVTVSS | |
| | ASTKGPSVFPLAPCSRSTSESTAAL | |
| | GCLVKDYFPEPVTVSWNSGALTSGV | |
| | HTFPAVLQSSGLYSLSSVVTVPSSS | |
| | LGTKTYTCNVDHKPSNTKVDKRVES | |
| | KYGPPCPPCPAPEAAGGPSVFLFPP | |
| | KPKDTLMISRTPEVTCVVVDVSQED | |
| | PEVQFNWYVDGVEVHNAKTKPREEQ | |
| | FNSTYRVVSVLTVLHQDWLNGKEYK | |
| | CKVSNKGLPSSIEKTSKAKGQPREP | |
| | QVYTLPPSQEEMTKNQVSLTCLVKG | |
| | FYPSDIAVEWESNGQPENNYKTTPP | |
| | VLDSDGSFLLYSKLTVDKSRWQEGN | |
| | VFSCSVMHEALHNHYTQKSLSLSLG | |
| | K (SEQ ID NO: 15) | |
| teclistamab | >Heavy chain 1 | >Light chain 1 |
| | QLQLQESGPGLVKPSETLSLTCTVS | SYVLTQPPSVSVAPGQTARITCGG |
| | GGSISSGSYFWGWIRQPPGKGLEWI | NNIGSKSVHWYQQPPGQAPVVVVY |
| | GSIYYSGITYYNPSLKSRVTISVDT | DDSDRPSGIPERFSGSNSGNTATL |
| | SKNQFSLKLSSVTAADTAVYYCARH | TISRVEAGDEAVYYCQVWDSSSDH |
| | DGAVAGLFDYWGQGTLVTVSSASTK | VVFGGGTKLTVLGQPKAAPSVTLF |
| | GPSVFPLAPCSRSTSESTAALGCLV | PPSSEELQANKATLVCLISDFYPG |
| | KDYFPEPVTVSWNSGALTSGVHTFP | AVTVAWKGDSSPVKAGVETTTPSK |
| | AVLQSSGLYSLSSVVTVPSSSLGTK | QSNNKYAASSYLSLTPEQWKSHRS |
| | TYTCNVDHKPSNTKVDKRVESKYGP | YSCQVTHEGSTVEKTVAPTECS |
| | PCPPCPAPEAAGGPSVFLFPPKPKD | (SEQ ID NO: 20) |
| | TLMISRTPEVTCVVVDVSQEDPEVQ | >Light chain 2 |
| | FNWYVDGVEVHNAKTKPREEQFNST | QTVVTQEPSLTVSPGGTVTLTCRS |
| | YRVVSVLTVLHQDWLNGKEYKCKVS | STGAVTTSNYANWVQQKPGQAPRG |
| | NKGLPSSIEKTISKAKGQPREPQVY | LIGGTNKRAPGTPARFSGSLLGGK |
| | TLPPSQEEMTKNQVSLTCLVKGFYP | AALTLSGVQPEDEAEYYCALWYSN |
| | SDIAVEWESNGQPENNYKTTPPVLD | LWVFGGGTKLTVLGQPKAAPSVTL |
| | SDGSFFLYSRLTVDKSRWQEGNVES | FPPSSEELQANKATLVCLISDFYP |
| | CSVMHEALHNHYTQKSLSLSLGK | GAVTVAWKADSSPVKAGVETTTPS |
| | (SEQ ID NO: 18) | KQSNNKYAASSYLSLTPEQWKSHR |
| | >Heavy chain 2 | SYSCQVTHEGSTVEKTVAPTECS |
| | EVQLVESGGGLVQPGGSLRLSCAAS | (SEQ ID NO: 21) |
| | GFTFNTYAMNWVRQAPGKGLEWVAR | |
| | IRSKYNNYATYYAASVKGRFTISRD | |
| | DSKNSLYLQMNSLKTEDTAVYYCAR | |
| | HGNFGNSYVSWFAYWGQGTLVTVSS | |
| | ASTKGPSVFPLAPCSRSTSESTAAL | |
| | GCLVKDYFPEPVTVSWNSGALTSGV | |
| | HTFPAVLQSSGLYSLSSVVTVPSSS | |
| | LGTKTYTCNVDHKPSNTKVDKRVES | |
| | KYGPPCPPCPAPEAAGGPSVFLFPP | |
| | KPKDTLMISRTPEVTCVVVDVSQED | |
| | PEVQFNWYVDGVEVHNAKTKPREEQ | |
| | FNSTYRVVSVLTVLHQDWLNGKEYK | |
| | CKVSNKGLPSSIEKTISKAKGQPRE | |
| | PQVYTLPPSQEEMTKNQVSLTCLVK | |
| | GFYPSDIAVEWESNGQPENNYKTTP | |
| | PVLDSDGSFLLYSKLTVDKSRWQEG | |
| | NVFSCSVMHEALHNHYTQKSLSLSL | |
| | GK (SEQ ID NO: 19) | |

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

By “biological sample” is meant a sample obtained from a subject. In some embodiments, a biological sample is a blood, sera, plasma, or bone marrow sample.

By “bortezomib” is meant a compound having the structure

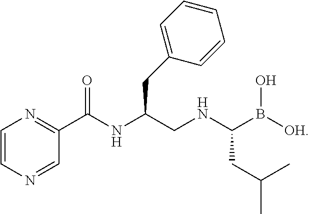 [SMILES: CC(C)C[C@H](NC[C@H](Cc1ccccc1)NC(=O)c1cnccn1)B(O)O]

and corresponding CAS No. 179324-69-7, or a pharmaceutically acceptable salt thereof.

By “Chimeric Antigen Receptor” or alternatively a “CAR” is meant a polypeptide capable of providing an immune effector cell with specificity for a target cell. In embodiments, the target cell is a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain. In some embodiments, the intracellular signaling domain contains a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain. In various embodiments, the intracellular signaling domain comprises two functional signaling domains derived from one or more costimulatory molecule(s), and a functional signaling domain derived from a stimulatory molecule. In one embodiment, the CAR comprises a chimeric fusion protein comprising an extracellular antigen binding domain, a transmembrane domain and an intracellular signaling domain comprising. In some cases, the intracellular signaling domain contains at least two functional signaling domains derived from one or more costimulatory molecule(s), and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

By “CAR-T cell” is meant a T cell modified to express a chimeric antigen receptor.

By “therapeutic agent” is meant an agent that inhibits cancer cell proliferation, inhibits cancer cell survival, increases cancer cell death, inhibits and/or stabilizes tumor growth, or that is otherwise useful in the treatment of cancer. In embodiments, therapeutic agents provided herein are used as part of an immunotherapy.

One of skill in the art can readily identify a therapeutic agent of use in a method for treating a cancer described herein (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L., Berkery R. (eds): Oncology Pocket Guide to Chemotherapy, 2nd ed. St. Louis, Mosby-Year Book, 1995; Fischer D.S., Knobf M. F., Durivage H. J. (eds): The Cancer Chemotherapy Handbook, 4th ed. St. Louis, Mosby-Year Book, 1993).

By “ciltacabtagene autoleucel” or “idecabtagene vicleucel (ide-cel)” is meant a commercially-available T cell expressing a chimeric antigen receptor capable of binding a B-cell maturation antigen (BCMA).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

As used herein, the term “coverage” refers to the percentage of genome covered by reads.

In one embodiment, low coverage or ultra-low pass coverage is less than about 1×. In some embodiments, high coverage is greater than 1×, 2×, 3×, 4×, 5×, 10×, 15×, 20×, 25×, 50×, 75×, 100×, 150×, 200×, 250×, 300×, 350×, 400×, 450×, 500×, 1000×, or higher. Coverage also refers to, in shotgun sequencing, the average number of reads representing a given nucleotide in the reconstructed sequence. It can be calculated from the length of the original genome (G), the number of reads (N), and the average read length (L) as N*(L/G). Biases in sample preparation, sequencing, and genomic alignment and assembly can result in regions of the genome that lack coverage (that is, gaps) and in regions with much higher coverage than theoretically expected. It is important to assess the uniformity of coverage, and thus data quality, by calculating the variance in sequencing depth across the genome. The term depth may also be used to describe how much of the complexity in a sequencing library has been sampled. All sequencing libraries contain finite pools of distinct DNA fragments. In a sequencing experiment, only some of these fragments are sampled. “Detect” refers to identifying the presence, absence, or amount of the analyte to be detected. In various embodiments, the analyte is one or more of the markers listed herein.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “dexamethasone” is meant a compound having the structure

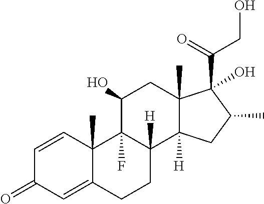

and corresponding to CAS No. 50-02-2, or a pharmaceutically acceptable salt thereof.

By “disease” is meant any condition or disorder that damages or interferes with the normal function of a cell, tissue, or organ. In some instances, the disease is a hematological malignancy. Examples of diseases include multiple myeloma (MM) and MM precursor diseases, such as smoldering multiple myeloma (SMM) and monoclonal gammopathy of undetermined significance (MGUS). In some embodiments, the MM is a plasma cell leukemia, which is a rare aggressive form of MM.

By “effective amount” is meant the amount of an agent required to ameliorate the symptoms of a disease of a patient relative to an untreated patient having the disease. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The disclosure provides a number of targets that are useful for the development of highly specific drugs to treat, or a disorder characterized by the methods delineated herein. In addition, the methods of the disclosure provide a route for analyzing any number of compounds for effects on a disease described herein with high-volume throughput, high sensitivity, and low complexity.

By “FAM46C polypeptide” is meant a polypeptide having at least about 85% amino acid sequence identity to GenBank Accession No. EAW56679.1, which is provided below, or a functional fragment thereof. In various embodiments, the FAM46C polypeptide is capable of enhancing replication of a yellow fever virus or a Venezuelan equine encephalitis virus. >EAW56679.1 family with sequence similarity 46, member C [Homo sapiens]

(SEQ ID NO: 22)
MAEESSCTRDCMSFSVLNWDQVSRLHEVLTEVVPIHGRGNFPTLEITLKD
IVQTVRSRLEEAGIKVHDVRLNGSAAGHVLVKDNGLGCKDLDLIFHVALP
TEAEFQLVRDVVLCSLLNFLPEGVNKLKISPVTLKEAYVQKLVKVCTDTD
RWSLISLSNKNGKNVELKFVDSIRRQFEFSVDSFQIILDSLLFFYDCSNN
PISEHFHPTVIGESMYGDFEEAFDHLQNRLIATKNPEEIRGGGLLKYSNL
LVRDERPTDQEEIKTLERYMCSRFFIDFPDILEQQRKLETYLQNHFAEEE
RSKYDYLMILRRVVNESTVCLMGHERRQTLNLISLLALRVLAEQNIIPSA
TNVTCYYQPAPYVSDGNESNYYVAHPPVTYSQPYPTWLPCN.

By “FAM46C polynucleotide” is meant a nucleic acid molecule that encodes an FAM46C polypeptide as well as the introns, exons, 3′ untranslated regions, 5′ untranslated regions, and regulatory sequences associated with its expression, or fragments thereof. A representative FAM46C polynucleotide sequence is provided below (GenBank Accession No. CH471122.1). A representative FAM46C gene sequence is provided at Ensembl Accession No. ENSG00000183508.

>CH471122.1:9111361-9112536 Homo sapiens 211000035831403 genomic scaffold, whole genome shotgun sequence

(SEQ ID NO: 23)
ATGGCAGAGGAGAGCAGCTGTACCAGGGATTGCATGTCCTTCAGCGTGCT
CAACTGGGATCAGGTTAGCCGGCTGCATGAGGTCCTCACTGAAGTTGTAC
CTATCCACGGACGAGGCAACTTTCCAACCTTGGAGATAACTCTGAAGGAC
ATCGTCCAGACCGTCCGCAGTCGGCTGGAGGAGGCAGGCATCAAAGTGCA
CGACGTCCGGCTGAATGGCTCCGCAGCTGGCCACGTTTTGGTCAAAGACA
ATGGCTTGGGCTGCAAAGACCTGGACCTAATCTTCCATGTGGCTCTTCCA
ACAGAGGCAGAATTTCAGCTGGTTAGAGATGTGGTTCTGTGTTCCCTTCT
GAACTTCCTGCCAGAGGGTGTGAACAAGCTCAAAATCAGTCCAGTCACTC
TGAAGGAGGCATATGTGCAGAAGCTAGTGAAGGTTTGCACGGACACTGAC
CGCTGGAGCCTGATCTCCCTCTCCAACAAGAACGGGAAGAACGTGGAGCT
GAAGTTTGTCGACTCCATTCGGCGTCAGTTTGAGTTCAGTGTGGACTCTT
TCCAAATCATCCTGGATTCTTTGCTTTTCTTCTATGACTGTTCCAATAAT
CCCATCTCTGAGCACTTCCACCCCACCGTGATTGGGGAGAGCATGTACGG
GGACTTTGAGGAAGCTTTTGACCATCTGCAGAACAGACTGATCGCCACCA
AGAACCCAGAAGAAATCAGAGGCGGGGGACTTCTCAAGTACAGCAACCTT
CTTGTGCGGGACTTCAGGCCCACAGACCAGGAAGAAATCAAAACTCTAGA
GCGCTACATGTGCTCCAGGTTCTTCATCGACTTCCCGGACATCCTTGAAC
AGCAGAGGAAGTTGGAGACTTACCTTCAAAACCACTTCGCTGAAGAAGAG
AGAAGCAAGTACGACTACCTCATGATCCTTCGCAGGGTGGTGAACGAGAG
CACCGTGTGTCTCATGGGGCATGAACGCAGGCAGACTCTGAACCTCATCT
CCCTCCTGGCCTTGCGTGTGCTGGCGGAACAAAACATCATCCCCAGTGCC
ACCAACGTCACCTGTTACTACCAGCCGGCCCCTTACGTCAGTGATGGCAA
CTTCAGCAACTACTACGTTGCCCATCCTCCAGTCACCTACAGCCAGCCTT
ACCCTACCTGGCTGCCCTGTAACTAA.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. “Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “iberdomide” is meant a compound having the structure

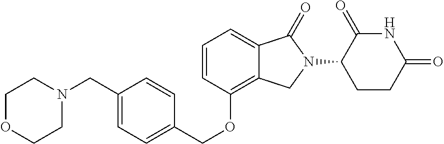 [SMILES: O=C1CC[C@H](N2Cc3c(OCc4ccc(CN5CCOCC5)cc4)cccc3C2=O)C(=O)N1]

and corresponding to CAS No. 1323403-33-3, or a pharmaceutically acceptable salt thereof.

By “increase” is meant to alter positively relative to a reference. An increase may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from an original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding an additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “KRAS polypeptide” is meant a polypeptide having at least about 85% amino acid sequence identity to GenBank Accession No. AAB41942.1, which is provided below, or a fragment thereof capable of binding GDP/GTP and acting as an intracellular signal transducer. >AAB41942.1 K-ras oncogene protein [Homo sapiens]

(SEQ ID NO: 24)
MTEYKLVVVGACGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
GVDDAFYTLVREIRKHKEKMSKDGKKKKKKSKTKCVIM.

By “KRAS polynucleotide” is meant a nucleic acid molecule that encodes an KRAS polypeptide as well as the introns, exons, 3′ untranslated regions, 5′ untranslated regions, and regulatory sequences associated with its expression, or fragments thereof. A representative KRAS polynucleotide sequence is provided below (GenBank Accession No. M54968.1). A representative KRAS gene sequence is provided at Ensembl Accession No. ENSG00000133703. >M54968.1:193-759 Homo sapiens K-ras oncogene protein (KRAS) mRNA, complete cds

(SEQ ID NO: 25)
ATGACTGAATATAAACTTGTGGTAGTTGGAGCTTGTGGCGTAGGCAAGAG
TGCCTTGACGATACAGCTAATTCAGAATCATTTTGTGGACGAATATGATC
CAACAATAGAGGATTCCTACAGGAAGCAAGTAGTAATTGATGGAGAAACC
TGTCTCTTGGATATTCTCGACACAGCAGGTCAAGAGGAGTACAGTGCAAT
GAGGGACCAGTACATGAGGACTGGGGAGGGCTTTCTTTGTGTATTTGCCA
TAAATAATACTAAATCATTTGAAGATATTCACCATTATAGAGAACAAATT
AAAAGAGTTAAGGACTCTGAAGATGTACCTATGGTCCTAGTAGGAAATAA
ATGTGATTTGCCTTCTAGAACAGTAGACACAAAACAGGCTCAGGACTTAG
CAAGAAGTTATGGAATTCCTTTTATTGAAACATCAGCAAAGACAAGACAG
GGTGTTGATGATGCCTTCTATACATTAGTTCGAGAAATTCGAAAACATAA
AGAAAAGATGAGCAAAGATGGTAAAAAGAAGAAAAAGAAGTCAAAGACAA
AGTGTGTAATTATGTAA.

By “lenalidomide” is meant a compound having the structure

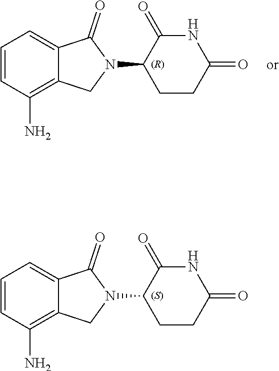

and corresponding to CAS No. 191732-72-6, or a pharmaceutically acceptable salt thereof.

By “MAF polypeptide” is meant a polypeptide having at least about 85% amino acid sequence identity to NCBI Ref. Seq. Accession No. XP_016878722.1, which is provided below, or a fragment thereof capable of controlling gene transcription. In various embodiments, MAF is used as a marker to identify the translocation of two chromosomes, where chromosome 14 resulting from the translocation contains IGH and chromosome 16 resulting from the translocation contains MAF.

>XP_016878722.1 transcription factor Maf isoform X1 [Homo sapiens]

(SEQ ID NO: 26)
MASELAMSNSDLPTSPLAMEYVNDFDLMKFEVKKEPVETDRIISQCGRLI
AGGSLSSTPMSTPCSSVPPSPSFSAPSPGSGSEQKAHLEDYYWMTGYPQQ
LNPEALGESPEDAVEALISNSHQLQGGFDGYARGAQQLAAAAGAGAGASL
GGSGEEMGPAAAVVSAVIAAAAAQSGAGPHYHHHHHHAAGHHHHPTAGAP
GAAGSAAASAGGAGGAGGGGPASAGGGGGGGGGGGGGGAAGAGGALHPHH
AAGGLHFDDRFSDEQLVTMSVRELNRQLRGVSKEEVIRLKQKRRTLKNRG
YAQSCRFKRVQQRHVLESEKNQLLQQVDHLKQEISRLVRERDAYKEKYEK
LVSSGFRENGSSSDNPSSPEFFMYPRESSTSVM.

By “MAF polynucleotide” is meant a nucleic acid molecule that encodes an MAF polypeptide as well as the introns, exons, 3′ untranslated regions, 5′ untranslated regions, and regulatory sequences associated with its expression, or fragments thereof. A representative MAF polynucleotide sequence is provided below (NCBI Ref. Seq. Accession No. XM_017023233.3). A representative MAF gene sequence is provided at Ensembl Accession No.

ENSG00000178573.

>XM_017023233.3:836-1987 PREDICTED: Homo sapiens MAF bZIP transcription factor (MAF), transcript variant X7, mRNA

(SEQ ID NO: 27)
ATGGCATCAGAACTGGCAATGAGCAACTCCGACCTGCCCACCAGTCCCCT
GGCCATGGAATATGTTAATGACTTCGATCTGATGAAGTTTGAAGTGAAAA
AGGAACCGGTGGAGACCGACCGCATCATCAGCCAGTGCGGCCGTCTCATC
GCCGGGGGCTCGCTGTCCTCCACCCCCATGAGCACGCCGTGCAGCTCGGT
GCCCCCTTCCCCCAGCTTCTCGGCGCCCAGCCCGGGCTCGGGCAGCGAGC
AGAAGGCGCACCTGGAAGACTACTACTGGATGACCGGCTACCCGCAGCAG
CTGAACCCCGAGGCGCTGGGCTTCAGCCCCGAGGACGCGGTCGAGGCGCT
CATCAGCAACAGCCACCAGCTCCAGGGCGGCTTCGATGGCTACGCGCGCG
GGGCGCAGCAGCTGGCCGCGGCGGCCGGGGCCGGTGCCGGCGCCTCCTTG
GGCGGCAGCGGCGAGGAGATGGGCCCCGCCGCCGCCGTGGTGTCCGCCGT
GATCGCCGCGGCCGCCGCGCAGAGCGGCGCGGGCCCGCACTACCACCACC
ACCACCACCACGCCGCCGGCCACCACCACCACCCGACGGCCGGCGCGCCC
GGCGCCGCGGGCAGCGCGGCCGCCTCGGCCGGTGGCGCTGGGGGCGCGGG
CGGCGGTGGCCCGGCCAGCGCTGGGGGCGGCGGCGGCGGCGGCGGCGGCG
GAGGCGGCGGGGGCGCGGCGGGGGGGGGGGGCGCCCTGCACCCGCACCAC
GCCGCCGGCGGCCTGCACTTCGACGACCGCTTCTCCGACGAGCAGCTGGT
GACCATGTCTGTGCGCGAGCTGAACCGGCAGCTGCGCGGGGTCAGCAAGG
AGGAGGTGATCCGGCTGAAGCAGAAGAGGCGGACCCTGAAAAACCGCGGC
TATGCCCAGTCCTGCCGCTTCAAGAGGGTGCAGCAGAGACACGTCCTGGA
GTCGGAGAAGAACCAGCTGCTGCAGCAAGTCGACCACCTCAAGCAGGAGA
TCTCCAGGCTGGTGCGCGAGAGGGACGCGTACAAGGAGAAATACGAGAAG
TTGGTGAGCAGCGGCTTCCGAGAAAACGGCTCGAGCAGCGACAACCCGTC
CTCTCCCGAGTTTTTCATGTACCCAAGGGAATCCTCTACATCAGTGATGT
GA.

By “MAFB polypeptide” is meant a polypeptide having at least about 85% amino acid sequence identity to GenBank Accession No. AAD30106.1, which is provided below, or a fragment thereof capable of controlling gene transcription. In various embodiments, MAFB is used as a marker to identify the translocation of two chromosomes, where chromosome 14 resulting from the translocation contains IGH and chromosome 20 resulting from the translocation contains MAFB.

>AAD30106.1 MAFB/Kreisler basic region/leucine zipper transcription factor [Homo sapiens]

(SEQ ID NO: 28)
MAAELSMGPELPTSPLAMEYVNDEDLLKEDVKKEPLGRAERPGRPCTRLQ
PVGSVSSTPLSTPCSSVPSSPSFSPTEQKTHLEDLYWMASNYQQMNPEAL
NLTPEDAVEALIGSHPVPQPLQSFDSFRGAHHHHHHHHPHPHHAYPGAGV
AHDELGPHAHPHHHHHHQASPPPSSAASPAQQLPTSHPGPGPHATASATA
AGGNGSVEDRESDDQLVSMSVRELNRHLRGFTKDEVIRLKHKRRTLKNRG
YAQSCRYKRVQQKHHLENEKTQLIQQVEQLKQEVSRLARERDAYKVKCEK
LANSGFREAGSTSDSPSSPEFFL.

By “MAFB polynucleotide” is meant a nucleic acid molecule that encodes an MAFB polypeptide as well as the introns, exons, 3′ untranslated regions, 5′ untranslated regions, and regulatory sequences associated with its expression, or fragments thereof. A representative MAFB polynucleotide sequence is provided below (GenBank Accession No. AF134157.1). A representative MAFB gene sequence is provided at Ensembl Accession No. ENSG00000204103. >AF134157.1:74-1045 Homo sapiens MAFB/Kreisler basic region/leucine zipper transcription factor (MAFB) mRNA, complete cds

(SEQ ID NO: 29_
ATGGCCGCGGAGCTGAGCATGGGGCCAGAGCTGCCCACCAGCCCGCTGGC
CATGGAGTATGTCAACGACTTCGACCTGCTCAAGTTCGACGTGAAGAAGG
AGCCACTGGGGCGCGCGGAGCGTCCGGGCAGGCCCTGCACACGCCTGCAG
CCAGTCGGCTCGGTGTCCTCCACACCGCTCAGCACTCCGTGTAGCTCCGT
GCCCTCGTCGCCCAGCTTCAGCCCGACCGAACAGAAGACACACCTCGAGG
ATCTGTACTGGATGGCGAGCAACTACCAGCAGATGAACCCCGAGGCGCTC
AACCTGACGCCCGAGGACGCGGTGGAAGCGCTCATCGGCTCGCACCCAGT
GCCACAGCCGCTGCAAAGCTTCGACAGCTTTCGCGGCGCTCACCACCACC
ACCATCACCACCACCCTCACCCGCACCACGCGTACCCGGGCGCCGGCGTG
GCCCACGACGAGCTGGGCCCGCACGCTCACCCGCACCATCACCATCATCA
CCAAGCGTCGCCGCCGCCGTCCAGCGCCGCTAGCCCGGCGCAACAGCTGC
CCACTAGCCACCCCGGGCCCGGGCCGCACGCGACGGCCTCGGCGACGGCG
GCGGGCGGCAACGGCAGCGTGGAGGACCGCTTCTCCGACGACCAGCTCGT
GTCCATGTCCGTGCGCGAGCTGAACCGCCACCTGCGGGGCTTCACCAAGG
ACGAGGTGATCCGCCTGAAGCACAAGCGGCGGACCCTGAAGAACCGGGGC
TACGCCCAGTCTTGCAGGTATAAACGCGTCCAGCAGAAGCACCACCTGGA
GAATGAGAAGACGCAGCTCATTCAGCAGGTGGAGCAGCTTAAGCAGGAGG
TGTCCCGGCTGGCCCGCGAGAGAGACGCCTACAAGGTCAAGTGCGAGAAA
CTCGCCAACTCCGGCTTCAGGGAGGCGGGCTCCACCAGCGACAGCCCCTC
CTCTCCCGAGTTCTTTCTGTGA.

By “mezigdomide” is meant a compound having the structure

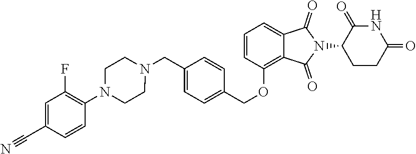 [SMILES: N#Cc1ccc(N2CCN(Cc3ccc(COc4cccc5c4C(=O)N([C@H]4CCC(=O)NC4=O)C5=O)cc3)CC2)c(F)c1]

and corresponding to CAS No. 2259648-80-9, or a pharmaceutically acceptable salt thereof.

By “marker” is meant any protein or polynucleotide having an alteration in structure, sequence, expression level, and/or activity that is associated with a developmental state, condition, disease, or disorder. In some embodiments, an alteration in a marker's expression level, concentration, abundance, activity or structure is detected. In some embodiments, a marker can include a genomic event, such as aneuploidy (e.g., hyperploidies, such as trisomies, or tetrasomies; and monoploidies), translocations, insertions or deletions, chromosomal arm gains or deletions, driver mutations, and/or mutations to a gene associated with multiple myeloma or a precursor thereof. The event detected can be predictive of risk and MM progression. In embodiments, a driver mutation is selected from one or more of t(14;16)(MAF), t(14;20)(MAFB), KRAS, NRAS, FAM46C, hyperdiploidy, gain(3q26.2), Del(1p), del(1p22.1), del(1p12), del(1p32), Gain(1q), gain(1q21.2), Del(4p16.3), Del(4q34.3), Del(8p), del(8p23.3), Del(8q24.21), gain(8q24.21), structural variation of MYC, Del(16q), del(16q12.1), and Del(17q21.2). In some embodiments, a driver mutation is selected from one or more of KRAS, NRAS, DIS3, FAM46C, BRAF, TP53, HIST1H1E, FGFR3, SP140, MAX, TRAF3, CYLD, PRDM1, DUSP2, IGLL5, IRF4, PTPN11, PRKD2, KLHL6, ATM, LTB, CCND1, TCLIA, SAMHDI, IKZF3, IKBKB, HISTIHIB, HNRNPU, DDX5, SMU1, BCL7A, RB1, RNASEH2C, PIM1, HNRNPA2B1, RPL5, EGRI, IDHI, ATP5D, SETD2, FRMPD2, FIPIL1, CDKN1B, EEF1A1, TRAF2, SGPP1, FAM196A, IRFI, ACTG1, TGDS, HIST1H1D, HISTIH4E, BTG1, ANKRD9, HOOK1, RASA2, SP3, NFKBIA, EFTUD2, KRT2, SF3B1, FKBP14, POT1, MCPH1, and CASP3.

By “MYC polypeptide” is meant a polypeptide having at least about 85% amino acid sequence identity to GenBank Accession No. AAA59880.1, which is provided below, or a fragment thereof capable of activating or repressing transcription.

>AAA59880.1 c-m c protein [Homo sapiens]

(SEQ ID NO: 30)
MPLNVSFTNRNYDLDYDSVQPYFYCDEEENFYQQQQQSELQPPAPSEDIW
KKFELLPTPPLSPSRRSGLCSPSYVAVTPFSLRGDNDGGGGSFSTADQLE
MVTELLGGDMVNQSFICDPDDETFIKNIIIQDCMWSGFSAAAKLVSEKLA
SYQAARKDSGSPNPARGHSVCSTSSLYLQDLSAAASECIDPSVVFPYPLN
DSSSPKSCASQDSSAFSPSSDSLLSSTESSPQGSPEPLVLHEETPPTTSS
DSEEEQEDEEEIDVVSVEKRQAPGKRSESGSPSAGGHSKPPHSPLVLKRC
HVSTHQHNYAAPPSTRKDYPAAKRVKLDSVRVLRQISNNRKCTSPRSSDT
EENVKRRTHNVLERQRRNELKRSFFALRDQIPELENNEKAPKVVILKKAT
AYILSVQAEEQKLISEEDLLRKRREQLKHKLEQLRNSCA.

By “MYC polynucleotide” is meant a nucleic acid molecule that encodes an MYC polypeptide as well as the introns, exons, 3′ untranslated regions, 5′ untranslated regions, and regulatory sequences associated with its expression, or fragments thereof. A representative MYC polynucleotide sequence is provided below (GenBank Accession No. AH002905.2). A representative MYC gene sequence is provided at Ensembl Accession No. ENSG00000136997. >AH002905.2:1490-2246,2660-3222 Homo sapiens c-myc protein (MYC) gene, complete cds, alternatively spliced

(SEQ ID NO: 31)
ATGCCCCTCAACGTTAGCTTCACCAACAGGAACTATGACCTCGACTACGA
CTCGGTGCAGCCGTATTTCTACTGCGACGAGGAGGAGAACTTCTACCAGC
AGCAGCAGCAGAGCGAGCTGCAGCCCCCGGCGCCCAGCGAGGATATCTGG
AAGAAATTCGAGCTGCTGCCCACCCCGCCCCTGTCCCCTAGCCGCCGCTC
CGGGCTCTGCTCGCCCTCCTACGTTGCGGTCACACCCTTCTCCCTTCGGG
GAGACAACGACGGCGGTGGCGGGAGCTTCTCCACGGCCGACCAGCTGGAG
ATGGTGACCGAGCTGCTGGGAGGAGACATGGTGAACCAGAGTTTCATCTG
CGACCCGGACGACGAGACCTTCATCAAAAACATCATCATCCAGGACTGTA
TGTGGAGCGGCTTCTCGGCCGCCGCCAAGCTCGTCTCAGAGAAGCTGGCC
TCCTACCAGGCTGCGCGCAAAGACAGCGGCAGCCCGAACCCCGCCCGCGG
CCACAGCGTCTGCTCCACCTCCAGCTTGTACCTGCAGGATCTGAGCGCCG
CCGCCTCAGAGTGCATCGACCCCTCGGTGGTCTTCCCCTACCCTCTCAAC
GACAGCAGCTCGCCCAAGTCCTGCGCCTCGCAAGACTCCAGCGCCTTCTC
TCCGTCCTCGGATTCTCTGCTCTCCTCGACGGAGTCCTCCCCGCAGGGCA
GCCCCGAGCCCCTGGTGCTCCATGAGGAGACACCGCCCACCACCAGCAGC
GACTCTGAGGAGGAACAAGAAGATGAGGAAGAAATCGATGTTGTTTCTGT
GGAAAAGAGGCAGGCTCCTGGCAAAAGGTCAGAGTCTGGATCACCTTCTG
CTGGAGGCCACAGCAAACCTCCTCACAGCCCACTGGTCCTCAAGAGGTGC
CACGTCTCCACACATCAGCACAACTACGCAGCGCCTCCCTCCACTCGGAA
GGACTATCCTGCTGCCAAGAGGGTCAAGTTGGACAGTGTCAGAGTCCTGA
GACAGATCAGCAACAACCGAAAATGCACCAGCCCCAGGTCCTCGGACACC
GAGGAGAATGTCAAGAGGCGAACACACAACGTCTTGGAGCGCCAGAGGAG
GAACGAGCTAAAACGGAGCTTTTTTGCCCTGCGTGACCAGATCCCGGAGT
TGGAAAACAATGAAAAGGCCCCCAAGGTAGTTATCCTTAAAAAAGCCACA
GCATACATCCTGTCCGTCCAAGCAGAGGAGCAAAAGCTCATTTCTGAAGA
GGACTTGTTGCGGAAACGACGAGAACAGTTGAAACACAAACTTGAACAGC
TACGGAACTCTTGTGCGTAA

By “NRAS polypeptide” is meant a polypeptide having at least about 85% amino acid sequence identity to GenBank Accession No. AAA60255.1, which is provided below, or a fragment thereof capable of regulating cell division.

>AAA60255.1 N-ras onco ene [Homo sapiens]

(SEQ ID NO: 32)
MTEYKLVVVGAGGVGKSALTIQLIQNHFVDEYDPTIEDSYRKQVVIDGET
CLLDILDTAGQEEYSAMRDQYMRTGEGFLCVFAINNSKSFADINLYREQI
KRVKDSDDVPMVLVGNKCDLPTRTVDTKQAHELAKSYGIPFIETSAKTRQ
GVEDAFYTLVREIRQYRMKKLNSSDDGTQGCMGLPCVVM

By “NRAS polynucleotide” is meant a nucleic acid molecule that encodes an NRAS polypeptide as well as the introns, exons, 3′ untranslated regions, 5′ untranslated regions, and regulatory sequences associated with its expression, or fragments thereof. A representative NRAS polynucleotide sequence is provided below (GenBank Accession No. AH002961.2). A representative NRAS gene sequence is provided at Ensembl Accession No. ENSG00000213281. >AH002961.2:21-131,252-430,551-710,811-930 Homo sapiens N-ras oncogene (NRAS) gene, complete cds

(SEQ ID NO: 33)
ATGACTGAGTACAAACTGGTGGTGGTTGGAGCAGGTGGTGTTGGGAAAAG
CGCACTGACAATCCAGCTAATCCAGAACCACTTTGTAGATGAATATGATC
CCACCATAGAGGATTCTTACAGAAAACAAGTGGTTATAGATGGTGAAACC
TGTTTGTTGGACATACTGGATACAGCTGGACAAGAAGAGTACAGTGCCAT
GAGAGACCAATACATGAGGACAGGCGAAGGCTTCCTCTGTGTATTTGCCA
TCAATAATAGCAAGTCATTTGCGGATATTAACCTCTACAGGGAGCAGATT
AAGCGAGTAAAAGACTCGGATGATGTACCTATGGTGCTAGTGGGAAACAA
GTGTGATTTGCCAACAAGGACAGTTGATACAAAACAAGCCCACGAACTGG
CCAAGAGTTACGGGATTCCATTCATTGAAACCTCAGCCAAGACCAGACAG
GGTGTTGAAGATGCTTTTTACACACTGGTAAGAGAAATACGCCAGTACCG
AATGAAAAAACTCAACAGCAGTGATGATGGGACTCAGGGTTGTATGGGAT
TGCCATGTGTGGTGATGTAA.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing, a disorder or condition.

By “polynucleotide” or “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often advantageous to use rather than native forms because of properties such as, for example, enhanced stability in the presence of nucleases.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects, the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively relative to a reference. A reduction may be by 1%, 5%, 10%, 25%, 30%, 50%, 75%, 100%, or more, or by 1.5-fold, 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In embodiments, a reference is a healthy subject, population, or cell, or the genome sequence of a healthy subject, population, or cell. In some cases, a reference is a subject prior to treatment or prior to a change in a treatment for a disease, such as multiple myeloma. In some cases, a reference is a subject, population, or cell, or the genome sequence of a healthy subject, population, or cell at a particular point in time (e.g., a time at which a treatment was stopped or begun or a time at which disease status of a subject was previously characterized).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

By “remission” is meant a subject having substantially no signs or symptoms of multiple myeloma. In embodiments, a multiple myeloma subject in remission shows little-to-no signs or symptoms of multiple myeloma and/or shows signs or symptoms of multiple myeloma similar to those observed in a healthy subject and/or a subject having a non-active multiple myeloma (e.g., MGUS or SMM).

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30 °C, of at least about 37 °C, or of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30 °C in 750 mM NaCl, 75 mM trisodium citrate, and 1% SDS. In another embodiment, hybridization will occur at 37 °C in 500 mM NaCl, 50 mM trisodium citrate, 1% SDS, 35% formamide, and 100 μg/ml denatured salmon sperm DNA (ssDNA). In another embodiment, hybridization will occur at 42 °C in 250 mM NaCl, 25 mM trisodium citrate, 1% SDS, 50% formamide, and 200 g/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25 °C, of at least about 42 °C, or of at least about 68 °C. In one embodiment, wash steps will occur at 25 °C in 30 mM NaCl, 3 mM trisodium citrate, and 0.1% SDS. In some embodiments, wash steps will occur at 42 °C in 15 mM NaCl, 1.5 mM trisodium citrate, and 0.1% SDS. In some cases, wash steps will occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid level to the sequence used for comparison. In various embodiments, a polypeptide or polynucleotide suitable for use in compositions or methods of the disclosure comprises an amino acid or polynucleotide sequence having about or at least about 85%, 90%, 95%, 96%, 97%, 98%, 99%, or greater sequence identity to a sequence provided herein.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. Numerical ranges, for example “between x and y” or “from x to y”, include the endpoint values of x and y. Also herein, the recitations of numerical ranges by endpoints include all numbers subsumed within that range as well as the endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

By “venetoclax” is meant a compound having the structure

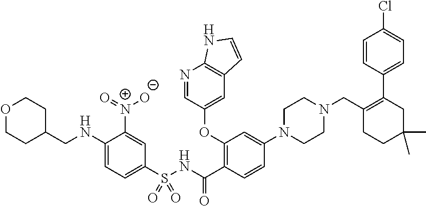 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

and corresponding to CAS No. 1257044-40-8, or a pharmaceutically acceptable salt thereof.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The symbol “>,” used in the context “x>y” indicates that the first number (e.g., x) is greater than the second (e.g., y). The symbol “≥,” used in the context “x≥y” indicates that the first number (e.g., x) is greater than or equal to the second (e.g., y). The symbol “<,” used in the context “x<y” indicates that the second number (e.g., y) is greater than the first (e.g., x). The symbol “≤,” used in the context “x≤y” indicates that the second number (e.g., y) is greater than or equal to the first (e.g., x).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments. Any compositions or methods provided herein can be combined with one or more of any of the other compositions and methods provided herein.

### BRIEF DESCRIPTION OF THE DRAWINGS

FIGS. 1A-1 to 1E provide a waterfall plot, violin plots, and Kaplan Meier curves showing the landscape of somatic mutations in monoclonal gammopathy of undetermined significance/smoldering multiple myeloma (MGUS/SMM) and a multiple myeloma-like (MM-like) signature to score disease development. FIGS. 1A-1 to 1A-5 (where FIGS. 1A-1 to 1A-5 are each continuations of each other, respectively, and FIG. 1A-5 provides the legend corresponding to FIGS. 1A-1 to 1A-4) provide a waterfall plot illustrating the genomic architecture of MM and its precursors MGUS/SMM across mutation classes (single nucleotide variants (SNVs), insertions/deletions (Indels), copy number alterations (CNAs), structural variants (SVs), and Translocations) from a comprehensive collection and harmonization of tumor next generation sequencing (NGS) data (n=1030 patients). Enrichment of association in precursors is shaded dark grey (−1), while those in MM are shaded light grey (+1) in the rightmost panel to define the “MM-like” signature score. FIG. 1B provides a violin plot showing “MM-like” signature scores distribution from MGUS to SMM to MM. FIG. 1C provides a violin plot showing “MM-like” signature scores distribution in the SMM cohort, subgrouped by International Myeloma Working Group (IMWG) 2/20/20 score. FIGS. 1D and 1E provide Kaplan Meier curves illustrating probability of progression of SMM to MM based on “MM-like” signature scoring in whole genome sequencing data (WGS) (FIG. 1D) and validated in an orthogonal dataset (FIG. 1E) (Bustoros, et. al., “Genomic Profiling of Smoldering Multiple Myeloma Identifies Patients at a High Risk of Disease Progression,” Journal of Clinical Oncology, 38:2380-90 (2020), doi: 10.1200/JCO.20.00437).

FIGS. 2A to 2C provide a swimmer plot, schematic diagrams, and stacked area plots showing application of the “MM-like” signature in serial tumor samples from patients. FIG. 2A provides a swimmer plot demonstrating the clinical efficacy of applying “MM-like” score in longitudinal sample setting up to 54 months from initial biopsy. Patients characterized by either stable disease trajectory (n=12, black radio button) or evolving genome (n=5, dark grey radio button) were identified, with increasing “MM-like” score driven by the acquisition of MM driver mutations (KRAS, NRAS, del13q, del1p). FIGS. 2B and 2C provide schematic diagrams and stacked area plots showing two examples of clinical relevance demonstrating the longitudinal impact of clinical whole-genome sequencing (WGS) in detecting a “MM-like” score and high-risk disease emergence in association with surrogate clinical biomarkers. FIG. 2B provides schematic diagrams and a stacked area plot showing “MGUS” monitoring over 3 years, with phylogenetic tree reconstruction to estimate clone driving growth and novel detection of Hyperdiploidy within a bi-allelic TP53 mutant context. FIG. 2C provides schematic diagrams and a stacked area plot showing “HR-SMM” monitoring over 2 years: phylogenetic analyses reveal newly acquired recurrent MM event of deletion 1p and SP140 identified and assigned to emerging clone (cluster 5). Concurrently, clinical M-spike levels show stable overall tumor burden.

FIGS. 3A to 3E provide plots, a schematic diagram, and box-and-whisker plots showing timing and cell of origin analyses of clonal and subclonal features of MGUS/SMM. FIG. 3A provides a plot showing clonal trisomies were used to estimate the age of emergence of the last clonal population in patients across the MM spectrum. FIG. 3B provides a box-and-whisker plot showing estimates of the molecular age across disease stages from MGUS (n=14), SMM (N=46), and MM (N=31). FIG. 3C provides a box-and-whisker plot showing estimates of the molecular age of the tumor in MGUS/SMM who progressed during the study course versus those who remained stable. FIG. 3D provides a schematic diagram and bar graph showing a cell of origin analysis (Kübler, et al., “Tumor mutational landscape is a record of the pre-malignant state,” bioRxiv. 2019:517565. doi: 10.1101/517565) showing mutation profile of MM and MGUS/SMM origin correlate with germinal center, memory B cells and plasma cells. FIG. 3E provides a plot showing Bradley-Terry scores estimates from the clonality league competition in MGUS/SMM (grey and closed circles) and multiple myeloma (black and open circles). Grey dots on the right represent events more likely to be subclonal in MGUS/SMM than in MM.

FIGS. 4A to 4E provide stacked bar graphs, a circus plot, and a bar graph showing mutational processes and structural variation in MGUS/SMM. FIG. 4A provides a stacked bar graph showing the proportion of mutational signatures obtained with the hdp algorithm, for whole genomes (top row), restricted to clustered mutations only (within 1,000 bases of another, middle row), and within immunoglobulin loci (bottom row). Annotation from COSMIC reference v3. FIG. 4B provides a stacked bar graph showing relative frequency of mutational processes in whole-genomes (FIG. 4B, top row), close to structural variants (SV) breakpoints (<10,000 base pairs, FIG. 4B, middle row), and in significant SV drivers (FIG. 4B, bottom row). FIG. 4C provides a stacked bar graph showing for each SV driver and known canonical myeloma translocations (MMSET, CCND1, WWOX/MAF, MYC), frequency of mutational processes close to SV breakpoints (<10,000 base pairs). FIG. 4D provides a circus plot depicting partners of MYC (chromosome 8) in MGUS/SMM, with recurrent partners (>3 patients) shown in dark grey. FIG. 4E provides a bar graph showing the fraction of patients with deep whole genome sequencing (WGS) performed and MYC translocation detected (immunoglobulin and nonimmunoglobulin).

FIGS. 5A to 5E provide a filtered heatmap, a schematic diagram, a plot, and box-and-whisker plots showing hotspots and candidate drivers in non-coding gene elements. FIG. 5A provides a filtered heatmap representing significantly hypermutated non-coding gene and regulatory elements detected with the DIG algorithm on MGUS/SMM/MM participants (overall and IMWG subgroup in different shades of grey). Left panel: Fraction of normal B cell expansions with mutations in the same elements (data from Machado, et al., “Diverse mutational landscapes in human lymphocytes,” Nature, 608:724-732 (2022)). Middle-right panel: mutational signature weights (including APOBEC and AID) in each element. Right panel: q score for each element hypermutation in MGUS/SMM/MM. FIG. 5B provides a schematic diagram showing the ILF2 promoter element, genomic coordinates, counts, and tumor allele identified in the Examples provided herein. FIG. 5C provides a plot showing cancer cell fraction (CCF) estimates from ABSOLUTE for each mutant allele of the Examples provided herein. FIGS. 5D and 5E provide box-and-whisker plots showing ILF2 gene expression levels by mutation status (FIG. 5D, 1^{st} hotspot, FIG. 5E, 2^{nd} hotspot) and Gain(1q21.2) status in newly diagnosed MM from the CoMMpass study.

FIGS. 6A and 6B present plots demonstrating that MM-like score correlated with overall survival (OS) and progression-free survival (PFS) in newly diagnosed multiple myeloma (MM).

FIGS. 7A to 7C shows a comparison of the MM-like scores, progression, and 2/20/20 risk scores for multiple cohorts of patients. (A) Kaplan-Meier curves from progression-free survival in SMM from an external validation cohort (N=77, Boyle et al.) with deep target sequencing panels (copy number abnormalities excluded, HR=1.8, CI_{95%}=[1.1-3.0], P=0.03). (B) Hazard ratios and 95% confidence intervals for a Cox regression to model time to progression with MM-like score >1 and 20/2/20 risk system stratified by study of origin (n=225). (C) Hazard ratios and 95% confidence intervals from the Cox proportional hazard models for progression to MM with MM-like score (>1 versus ≤1) and 20/2/20 clinical risk stratification (reference: low) as predictors, for this study, Bustoros et al., and Boyle et al. (minus two patients without 20/2/20 risk available). The central point and error bars represent the 15 Hazard ratio and its 95% confidence interval from the model described. Significance levels *: P<0.05; **: P<0.01; ***: P<0.001; ****: P<0.0001.)

### DETAILED DESCRIPTION

The disclosure features methods for treating a subject having a symptomatic multiple myeloma or a precursor thereof, where the methods involve characterizing a plasma cell dyscrasia (e.g., monoclonal gammopathy of undetermined significance, smoldering multiple myeloma, multiple myeloma, and plasma cell leukemia) in a biological sample from a subject as being high, medium, or low risk by assigning a multiple myeloma (MM)-like score (sometimes referred to simply as “MM-score”) to a plasma cell dyscrasia. In some cases, the methods involve sequencing DNA from the biological sample using whole-genome sequencing (WGS).

Multiple myeloma (MM) and its precursor stages, monoclonal gammopathy of undetermined significance (MGUS) and smoldering multiple myeloma (SMM), are clinically well defined; however, few reliable strategies exist to capture patients at risk of progression to overt MM. Common methods used to identify patients at risk of progression from asymptomatic, precursor stages of Multiple Myeloma do not include somatic alterations that play a role in the disease. The present disclosure is based, at least in part, upon the development of a method for assessing patient risk for progression to overt MM that include analysis of somatic alterations that play a role in the disease. A comprehensive collection of MM genomic data containing 1,030 patients (218 MGUS/SMM) was used to identify recurrent coding and non-coding candidate drivers, as well as significant hotspots of structural variation. Those drivers were used to define and validate a simple “MM-like” score that associates with disease stages (MGUS to MM), risk classification (low to high risk SMM), subclonal outgrowth (with repeated whole-genome sequencing over time), and time to progression (SMM to overt MM). The MM genomic map provided new insights on the time of initiation and cell of origin of the disease, on the order of acquisition of genomic alterations, and on mutational processes found across stages of the transformation. The newly developed “myeloma-like” genomic signature serves as a tumor-based biomarker of precursors for the prediction and higher probability of progression from MGUS/SMM to overt MM. Taken together, the present disclosure demonstrates the potential of clinical genome sequencing to better inform progression risk, monitoring, and early intervention strategies. Among individuals with the intermediate SMM stage, the MM-like score increased from low, to intermediate, to high-risk subgroups. In individuals with survival data, the MM-like score independently correlated with a higher likelihood of progressing to symptomatic cancer. Accordingly, the present disclosure features methods for characterizing plasma cell dyscrasia in a subject to assist in selecting a treatment for the subject.

### Hematological Malignancies

Multiple myeloma (MM) is a plasma cell dyscrasia. Plasma cell dyscrasias are cancers of the plasma cells. They are produced as a result of malignant proliferation of a monoclonal population of plasma cells that may secrete detectable levels of a monoclonal immunoglobulin or paraprotein commonly referred to as M protein. Other non-limiting examples of plasma cell dyscrasias include monoclonal gammopathy of undermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. MGUS, smoldering MM, and symptomatic MM represent a spectrum of the same disease, with MGUS being the least advanced and symptomatic MM being the most advanced.

Monoclonal Gammopathy of Undermined Significance (MGUS) MGUS is characterized by a serum monoclonal protein of less than 30 g/L, <10% plasma cells in the bone marrow, and the absence of end-organ damage. Asymptomatic MGUS stage consistently precedes multiple myeloma (MM). MGUS is present in 3% of persons >50 years and in 5%>70 years of age. The risk of progression to MM or a related disorder is 1% per year. Patients with (1) an abnormal serum free light chain ratio, (2) non-immunoglobulin G (IgG) MGUS, and (3) an elevated serum M protein (≥15 g/1) have a risk of progression at 20 years of 58%, compared with 37% among patients with two of the three aforementioned risk factors, 21% for those with one risk factor, and 5% for individuals with no risk factors. The cumulative probability of progression to active MM or amyloidosis is 51% at 5 years, 66% at 10 years and 73% at 15 years; the median time to progression was 4.8 years.

Smoldering Multiple Myeloma (SMM), also known as asymptomatic MM, is characterized by a serum immunoglobulin (Ig) G or IgA monoclonal protein of 30 g/L or higher and/or 10% or more plasma cells in the bone marrow but no evidence of end-organ damage. Not intending to be bound by theory, there are 2 different types of SMM: evolving smoldering MM and non-evolving Smoldering MM. Evolving SMM is characterized by a progressive increase in M protein and a shorter median time to progression (TTP) to active multiple myeloma of 1.3 years. Non-evolving SMM has a more stable M protein that can then change abruptly at the time of progression to active multiple myeloma, with a median TTP of 3.9 years.

Symptomatic or Active Multiple myeloma (MM) is a form of cancer that affects a type of white blood cell called the plasma cell. Multiple myeloma appears in the bone marrow, which is the soft tissue inside the bones that makes stem cells. In multiple myeloma, plasma cells, which mature from stem cells and typically produce antibodies to fight germs and other harmful substances, become abnormal. These abnormal cells are called myeloma cells. In 2021, an estimated 34,920 cases of multiple myeloma were diagnosed in the United States and over 12,410 patient deaths associated with multiple myeloma were reported. As the most common type of plasma cell cancer, effective treatment requires an accurate diagnosis and precise treatment.

In embodiments, symptomatic or active MM is characterized by any level of monoclonal protein and the presence of end-organ damage that consists of the CRAB criteria (hyperCalcemia, Renal insufficiency, Anemia, or Bone lesions). In some instances, multiple myeloma diagnosis is made using the detection of a biomarker for a myeloma defining event, as described, for example, in Rajkumar, S., et al., The Lancet Oncology, 15:E538-548 (2014), doi: 10.1016/S1470-2045(14)70442-5. MM is a plasma cell malignancy that characteristically involves extensive infiltration of bone marrow (BM), with the formation of plasmacytomas, as clusters of malignant plasma cells inside or outside of the BM milieu. Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia.

### Conventional Detection Methods

To date, the gold standard for characterizing MM disease state has required a bone marrow biopsy. In various embodiments, the methods of the disclosure involve sequencing polynucleotides obtained from a bone marrow biopsy. The methods of the disclosure are suitable for use alone, or if desired, may be used in concert with one or more conventional diagnostic methods.

Traditionally, the initial evaluation of a suspected hematological malignancy (e.g., a monoclonal gammopathy) included both serum and urine protein electrophoresis with immunofixation to identify and quantify the M protein. The majority of patients are expected to have a detectable M protein, but approximately 1-3% can present with a non-secretory myeloma that does not produce light or heavy chains. True non-secretory myeloma is thus rare, not least because, with the availability of serum free light chain testing, it is recognized that M protein is present. The most common M protein is IgG, followed by IgA, and light-chain-only disease. IgD and IgE are relatively uncommon and can be more difficult to diagnose because their M spikes are often very small. Up to 20% of patients will produce only light chains, which may not be detectable in the serum because they pass through the glomeruli and are excreted in the urine. The present disclosure provides methods that can also be used to detect and/or characterize a monoclonal gammopathy in a patient.

A standard evaluation of a documented monoclonal gammopathy includes a complete blood count with differential, calcium, serum urea nitrogen, and creatinine. Serum free light chain testing is also a useful diagnostic test (Piehler A. P. et al, Clin. Chem., 54: 1823-30 (2008)). Bone disease is best assessed by skeletal survey. Bone scans are not a sensitive measure of myelomatous bone lesions because the radioisotope is poorly taken up by lytic lesions in MM, as a result of osteoblast inhibition. Magnetic resonance imaging (MRI) is useful for the evaluation of solitary plasmacytoma of bone and for the evaluation of paraspinal and epidural components. 18F-FDG Positron Emission Tomography (PET)/CT scans are more sensitive in the detection of active lesions in the whole body (Fonti R. et al., J. Nucl. Meds, 49: 195-200 (2008)). Bone marrow aspiration and biopsy are helpful to quantify the plasma cell infiltrate and add important prognostic information with cytogenetic evaluation, including Fluorescent In Situ Hybridization (FISH). Additional prognostic information can be obtained with serum Beta-2 Microglobulin (%2M) and C-Reactive Protein (CRP).

Exemplary conventional criteria for the diagnosis of MM, SMM, and MGUS are detailed in Table 1 below. Distinction among these disease states may inform treatment decisions and prognostic recommendations.

TABLE 1
Conventional criteria for the diagnosis of MM, SMM, and MGUS

| Disorder | Disease definition |
|---|---|
| MGUS | Serum monoclonal protein level <3 g/dL, |
| | bone marrow plasma cells <10%, and |
| | absence of end-organ damage, such as lytic |
| | bone lesions, anemia, hypercalcemia, or renal |
| | failure, that can be attributed to a plasma cell |
| | proliferative disorder. |
| SMM | Serum monoclonal protein (IgG or IgA) level |
| | ≥3 g/dL and/or bone marrow plasma cells ≥10%, |
| | absence of end-organ damage, such as |
| | lytic bone lesions, anemia, hypercalcemia, or |
| | renal failure that can be attributed to a plasma |
| | cell proliferative disorder. |
| | Alternatively, or additionally, SMM may be |
| | defined by the absence of biomarkers (e.g., |
| | Myeloma Defining Event biomarkers), by |
| | bone-marrow plasma cell infiltration ≥60%, |
| | by serum-free light chain ratio ≥100, and/or |
| | by >1 focal lesion in the skeleton on |
| | magnetic resonance imaging analysis (see, |
| | Rajkumar SV, et al. International Myeloma |
| | Working Group updated criteria for the |
| | diagnosis of multiple myeloma. Lancet Oncol |
| | 15, e538-548 (2014)). In some instances, |
| | SMM may be associated with organ damage. |
| MM | Bone marrow plasma cells ≥10%, presence |
| | of serum and/or urinary monoclonal protein |
| | (except in patients with true nonsecretory |
| | multiple myeloma), plus evidence of lytic |
| | bone lesions, anemia, hypercalcemia, or renal |
| | failure that can be attributed to the underlying |
| | plasma cell proliferative disorder. |

Conventional staging systems involve the following. The most widely used myeloma staging system since 1975 has been the Durie-Salmon, in which the clinical stage of disease is based on several measurements including levels of M protein, serum hemoglobin value, serum calcium level, and the number of bone lesions. The International Staging System (ISS), developed by the International Myeloma Working Group is now also widely used (Greipp PR. et al, J. Clin. Oncol, 23:3412-20 (2005)). ISS is based on two prognostic factors: serum levels of B2M and albumin and is comprised of three stages: B2M 3.5 mg/L and albumin 3.5 g/dL (median survival, 62 months; stage I); B2M <3.5 mg/L and albumin <3.5 g/dL or B2M 3.5 to <5.5 mg/L (median survival, 44 months; stage II); and B2M 5.5 mg/L (median survival, 29 months; stage III). With an increased understanding of the biology of myeloma, other factors have been shown to correlate well with clinical outcome and are now commonly used. For example, cytogenetic abnormalities as detected by FISH techniques have been shown to identify patient populations with very different outcomes. For instance, loss of the long arm of chromosome 13 is found in up to 50% of patients and, when detected by metaphase chromosome analysis, is associated with poor prognosis. In addition, a hypodiploid karyotyped t(4;14), and -17p13.1 is typically associated with poor outcome, while the t(11; 14) and hypodiploidy are associated with improved survival (Kyrtsonis M. C. et al., Semin. Hematol, 46: 110-7, (2009)).

### Multiple Myeloma (MM)-Like Score (MM-Score)

The present disclosure features in various aspects a method for characterizing the severity of a cancer in a subject and/or risk for progression to symptomatic cancer from an asymptomatic state (e.g., MGUS or SMM) to inform the selection of a treatment for the subject. In various embodiments, the method involves collecting a biological sample from a subject (e.g., a bone marrow biopsy, a liquid biopsy, and/or a cancer cell or polynucleotides and/or polypeptides from a cancer cell) and determining whether or not any of the markers listed in each row of Table 4 are present in the biological sample. For each row of Table 4 containing one or more markers detected in the cancer cells of interest, the scores corresponding to each row were summed to determine the MM-like score. For example, if the biological sample contains the marker t(14;16)(MAF), a KRAS mutation, and an NRAS mutation, the biological sample is assigned an MM-like score of +1. It should be understood that when any one category includes more than one marker, a single category score (e.g., −1, +1) is assigned. While described herein using values of “1” when calculating an MM-like score, it should be understood that other values may be used to the same effect. In other words, the methods would effectively not change if a value of 10, rather than 1, were assigned according to detection of each marker, and the risk score criteria were similarly modified. In various embodiments, the subject has been diagnosed with MGUS, SMM, or MM.

In some embodiments, the methods of the disclosure involve determining whether or not a biological sample contains any of the following markers, or a subset thereof: t(14;16)(MAF), t(14;20)(MAFB), KRAS, NRAS, FAM46C, Hyperdiploidy, gain(3q26.2), Del(1p), del(1p22.1), del(1p12), del(1p32), Gain(1q), gain(1q21.2), Del(4p16.3), Del(4q34.3), Del(8p), del(8p23.3), Del(8q24.21), gain(8q24.21), structural variation of MYC, Del(16q), del(16q12.1), and Del(17q21.2). In some embodiments, the methods of the disclosure involve determining whether or not a biological sample contains any of the following markers or a subset thereof: t(14;16)(MAF), t(14;20)(MAFB), KRAS, NRAS, FAM46C, Hyperdiploidy, Del(1p), Gain(1q), Del(4p16.3), Del(4q34.3), Del(8p), structural variation of MYC, Del(16q), and Del(17q21.2). The markers may be detected according to any method described herein or otherwise available to one of skill in the art, or a combination thereof.

In various embodiments, an MM-like score of about or less than about −2, −1, 0, 1, or 2 is considered “low risk.” In some embodiments, an MM-like score of about 2, 3, and/or 4 is considered “intermediate risk.” In some cases, an MM-like score of about or greater than about 3, 4, 5, 6, or 7 is considered “high risk.” The MM-like scores corresponding to low risk, intermediate risk, and high risk are selected from among the values listed here such that they are non-overlapping. In some embodiments “low risk” corresponds to an MM-like score of between −2 and 2, “intermediate risk” corresponds to an MM-like score of between 3 and 4, and “high risk” corresponds to an MM-like score of between 5 and 12. In some embodiments “low risk” corresponds to an MM-like score of between −2 and 1, “intermediate risk” corresponds to an MM-like score of between 2 and 3, and “high risk” corresponds to an MM-like score of between 4 and 12.

### Types of Samples

This disclosure provides methods to extract and sequence a polynucleotide present in a sample. In one embodiment, the samples are biological samples generally derived from a human subject, such as a bodily fluid (such as ascites, blood, plasma, pleural fluid, serum, cerebrospinal fluid, phlegm, saliva, stool, urine, semen, prostate fluid, breast milk, or tears, or tissue sample (e.g., a tissue sample obtained by biopsy). In some embodiments, the biological sample is a bone marrow sample. In some embodiments, the samples are biological samples derived from an animal, such as a bodily fluid (such as blood, cerebrospinal fluid, phlegm, saliva, or urine) or tissue sample (e.g., a tissue sample obtained by biopsy). In some embodiments, the samples are biological samples from in vitro sources (such as cell culture medium). Cell free (cfDNA) attached to a substrate may be first suspended in a liquid medium, such as a buffer or a water, and then subject to sequencing and/or analysis. In some embodiments, the sample contains DNA within a cell, which may be extracted, sequenced and subject to the same analysis. In some embodiments, the sample is a biopsy (e.g., a needle biopsy) or a section. In some embodiments, the methods of the disclosure involve enriching a biological sample for tumor cells and subsequently characterizing the enriched tumor cells according to the methods provided herein.

### Treatments

Methods of inhibiting and/or treating cancer and tumors (e.g., a multiple myeloma) in a subject with cancer or a predisposition for developing cancer as identified by methods of the disclosure are also contemplated. Methods described herein are useful as clinical or companion diagnostics for therapies or can be used to guide treatment decisions based on clinical response/resistance.

In various embodiments, MM-like scores may be used to select a treatment strategy for a subject having MGUS or SMM as follows. A low MM-like score (e.g., −2 to 1) or intermediate MM-like score (e.g., 2 to 3) may indicate that the subject should be treated with close monitoring unless they are known to have high-risk SMM by other classification systems (e.g., 2/20/20 IMWG risk classification, PANGEA classification, or GEM/PETHEMA high risk classification).

A high MM-like score (e.g., >=4) may indicate that the subject should be enrolled in a clinical trial or administered one or more of the following treatments:
One or more BCMAxCD3 bispecific antibodies (such as teclistamab, elranatamab, and linvoseltamab);
One or more GPRC5DxCD3 bispecific antibodies (such as talquetamab);
A BCMA-directed CAR-T cell (such as ciltacabtagene autoleucel);
An immunomodulatory drug alone (e.g., for a fixed duration, such as 1 or 2 years) (such as lenalidomide);
An immunomodulatory agent (such as lenalidomide) in combination with a glucocorticoid (such as dexamethasone); and/or
An anti-CD38 monoclonal antibody (such as daratumumab or isatuximab) in combination with an immunomodulatory agent (such as lenalidomide) and a glucocorticoid (such as dexamethasone).

In some embodiments, MM-like scores may be used to select a treatment strategy for a subject who is newly diagnosed as having MM and found to be eligible for a transplant.

Treatment of a subject with a low to intermediate MM-like score may follow a standard risk protocol in absence of a high-risk disease and/or administer to the subject VRd (bortezomib, lenalidomide, and dexamethasone) or DRd (daratumumab, lenalidomide, and dexamethasone combined treatment), optionally with maintenance with lenalidomide, and/or an early or delayed autologous stem cell transplant (ASCT). Lenalidomide may be replaced with thalidomide or cyclophosphamide. Treatment of a subject with a high-risk MM-like score (e.g., >=4 or >=5) may follow a high-risk myeloma protocol, such as D-RVd (daratumumab, lenalidomide, bortezomib, and dexamethasone). Bortezomib may be replaced with carfilzomib. Currently high-risk cytogenetic abnormalities are defined by FISH positive for t(4;14), t(14;16); t(14;20), gain(1q), or del(17p). In some embodiments, a subject having a high-risk MM-like score may be enrolled in a clinical trial or administered one or more of the following treatments:
One or more BCMAxCD3 bispecific antibodies (such as teclistamab, elranatamab, and linvoseltamab);
One or more GPRC5DxCD3 bispecific antibodies (such as talquetamab);
One or more BCMA-targeted CAR-T cell therapies (ciltacabtagene autoleucel, idecabtagene vicleucel);
A combination of the above with a bispecific antibody;
Combinations of the above with replacement of lenalidomide with iberdomide or mezigdomide; and/or
Venetoclax for subjects detected as containing the marker t(11;14).

In some embodiments, MM-like scores may be used to select a treatment strategy for a subject who is newly diagnosed multiple myeloma and found to be transplant-ineligible as follows. A subject having a low or intermediate MM-like score may be treated with VRd (bortezomib, lenalidomide, and dexamethasone) or DRd (daratumumab, lenalidomide, and dexamethasone combined treatment), optionally with maintenance with lenalidomide. A subject having a high MM-like score may be treated with VRd (bortezomib, lenalidomide, and dexamethasone) or DRd (daratumumab, lenalidomide, and dexamethasone combined treatment), optionally with maintenance with lenalidomide and/or bortezomib. In some embodiments, a subject having a high-risk MM-like score may be enrolled in a clinical trial or administered one or more of the following treatments:
One or more BCMAxCD3 bispecific antibodies (such as teclistamab, elranatamab, and linvoseltamab);
One or more GPRC5DxCD3 bispecific antibodies (such as talquetamab);
One or more BCMA-targeted CAR-T cell therapies (ciltacabtagene autoleucel, idecabtagene vicleucel);
An immunomodulatory agent alone (e.g., for a fixed duration, such as 1 or 2 years) (such as lenalidomide);
A combination of an immunomodulatory agent (such as lenalidomide) and a glucocorticoid (such as dexamethasone);
A combination of an anti-CD38 monoclonal antibody (such as daratumumab or isatuximab) with an immunomodulatory (such as lenalidomide) and a glucocorticoid (such as dexamethasone);
Combinations of the above with a bispecific antibody;
Combinations of the above, with replacement of lenalidomide with iberdomide or mezigdomide; and/or
Venetoclax for subjects detected as containing the marker t(11;14).

Typically, a subject having a multiple myeloma is transplant eligible if organ functions are good (e.g., injection fraction >50%; pulmonary function tests, such as FEV1 and DLCO, each >50%, and good performance status).

Frontline therapy for MM typically includes either conventional chemotherapy or high-dose chemotherapy (HDT) supported by autologous or allogeneic stem cell transplantation (SCT), depending on patient characteristics such as performance status, age, availability of a sibling donor, comorbidities, and, in some cases, patient and physician preferences. Other treatments include: bortezomib, thalidomide, lenalidomide, dexamethasone, cyclophosphamide, melphalan, and stem cell transplant. For a patient under 70 years of age, autologous stem cell transplant is typically proposed after induction.

Non-limiting examples of agents suitable for use to treat a multiple myeloma include a therapeutic agent, such as a chemotherapeutic agent, radiation, or immunotherapy. Any suitable therapeutic treatment for a particular cancer may be administered. Examples of therapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (TAXOL), pilocarpine, prochloroperazine, rituximab, tamoxifen, taxol, topotecan hydrochloride, trastuzumab, vinblastine, vincristine and vinorelbine tartrate. Further non-limiting examples of therapeutic agents include an alkylating agent (e.g. busulfan, chlorambucil, cisplatin, cyclophosphamide (Cytoxan), dacarbazine, ifosfamide, mechlorethamine (mustargen), and melphalan), a topoisomerase inhibitor, an antimetabolite (e.g. 5-fluorouracil (5-FU), cytarabine (Ara-C), fludarabine, gemcitabine, and methotrexate), an anthracycline, an antitumor antibiotic (e.g. bleomycin, dactinomycin, daunorubicin, doxorubicin (Adriamycin), and idarubicin), an epipodophyllotoxin, nitrosureas (e.g. carmustine and lomustine), topotecan, irinotecan, doxorubicin, etoposide, mitoxantrone, bleomycin, busultan, mitomycin C, cisplatin, carboplatin, oxaliplatin and docetaxel.

In embodiments, response to therapy is measured by a reduction in M protein levels in serum and/or urine and the reduction in size or disappearance of plasmacytomas. The international uniform response criteria for MM have expanded upon the European Group for Blood and Marrow Transplantation criteria to provide a more comprehensive evaluation system (Dure B. G. et al., Leukemia, 20: 1467-73 (2006)). Meeting response criteria has been associated with improved survival in SCT trials with high-dose therapy. Similarly, time to progression (TTP) has been shown to be an important surrogate for improved survival. Despite high response rates to frontline therapy, virtually all patients eventually relapse. Table 2 shows the international uniform response criteria for MM.

TABLE 2
International uniform response criteria for multiple myeloma (MM)

| Response | |
|---|---|
| Subcategory | Response Criteria |
| CR (complete | Negative immunofixation on the serum and urine and disappearance of |
| response) | any soft tissue plasmacytomas and ≤5% plasma cells in bone marrow |
| sCR (stringent | CR as described above, plus: |
| complete response) | normal free light chain (FLC) ratio and absence of clonal cells in bone |
| | marrow by immunohistochemistry or immunofluorescence |
| VGPR (very good | Serum and urine M-protein detectable by immunofluorescence but not |
| partial response) | on electrophoresis or 90% or greater reduction in serum M-protein plus |
| | urine M-protein level <100 mg per 24 hours |
| PR (partial | >50% reduction of serum M-protein and reduction in 24-h urinary M- |
| response) | protein by >90% or to <200 mg per 24 h |
| | If the serum and urine M-protein are unmeasurable, a ≥50% decrease |
| | in the difference between involved and uninvolved FLC levels is |
| | required in place of the M-protein criteria |
| | If serum and urine M-protein are unmeasurable, and serum free light |
| | assay is also unmeasurable, ≥50% reduction in plasma cells is required |
| | in place of M-protein, provided baseline bone marrow plasma cell |
| | percentage was ≥30% |
| | In addition to the above listed criteria, if present at baseline, a ≥50% |
| | reduction in the size of soft tissue plasmacytomas is also required. |
| SD (stable disease) | Not meeting criteria for CR, VGPR, PR or progressive disease |

In embodiments, the subject has been diagnosed with cancer or is at risk of developing cancer, such as a multiple myeloma.

For therapeutic use, administration of an agent can begin at the detection of and/or surgical removal of tumors. Treatment can be followed by boosting doses until at least symptoms are substantially abated and for a period thereafter.

Some of the pharmaceutical compositions for therapeutic treatment described herein are intended for parenteral, topical, nasal, oral or local administration. In some embodiments, the pharmaceutical compositions are administered parenterally, e.g., intravenously, subcutaneously, intradermally, or intramuscularly. The disclosure provides compositions for parenteral administration which comprise a solution of a suitable agent dissolved or suspended in an acceptable carrier, such as, for example, an aqueous carrier. A variety of aqueous carriers may be used, e.g., water, buffered water, saline, glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an advantageous embodiment, the cancer therapeutic is an immunotherapeutic (e.g., an antibody). The cancer therapeutic can be a chimeric antigen receptor (CAR) T cell. The immunotherapeutic may be a cytokine therapeutic (such as an interferon or an interleukin), a dendritic cell therapeutic or an antibody therapeutic, such as a monoclonal antibody. In a particularly advantageous embodiment, the immunotherapeutic is a neoantigen (see, e.g., U.S. Pat. No. 9,115,402 and U.S. Pat. App. Pub. Nos. 2011/0293637, 2016/0008447, 2016/0101170, 2016/0331822 and 2016/0339090).

### Detection of Markers

The present disclosure provides for the detection of a variety of clinical variables associated with MM and/or an MM precursor disease (e.g., MGUS, SMM) for use in characterizing a plasma cell dyscrasia according to the methods provided herein. In some embodiments, the clinical variable is the level or presence of a marker, which may be a genetic marker (e.g., a mutation, a SNV), or a chemical marker (e.g., a biomolecule detected in the blood). Markers include but are not limited to creatinine, hemoglobin, serum M-protein, serum Free Light Chain (FLC) ratio, Bone Marrow Plasma Cell percent (BMPC %), total protein, IgA, IgM, IgG, kappa FLC), lambda FLC, calcium, albumin, LDH, beta-2 microglobulin, M-spike, LDH, beta-2 microglobulin, urine M-protein. Further non-limiting examples of markers include t(14;16)(MAF), t(14;20)(MAFB), KRAS, NRAS, FAM46C, Hyperdiploidy, gain(3q26.2), Del(1p), del(1p22.1), del(1p12), del(1p32), Gain(1q), gain(1q21.2), Del(4p16.3), Del(4q34.3), Del(8p), del(8p23.3), Del(8q24.21), gain(8q24.21), structural variation of MYC, Del(16q), del(16q12.1), and Del(17q21.2). In some embodiments, a level or the presence of a marker is detected at a single time point or serial values are annotated over time (e.g., days, weeks, months). In some embodiments, levels of a marker are detected at 3, 4, 5, 6, 7, 8, 9, 10, 11, 12, 16, or 24-month time intervals from the date of MGUS or SMM diagnosis. In some embodiments, levels of a marker described herein are detected prior to, during, or following initiation of a treatment. In some embodiments, levels of a marker are detected in a biological sample (e.g., blood, serum, plasma, or bone marrow biopsy).

The markers of this disclosure can be detected by any suitable method. The methods described herein can be used individually or in combination for a more accurate detection of the markers (e.g., biochip in combination with mass spectrometry, immunoassay in combination with mass spectrometry, single cell RNA sequencing, and the like).

One of skill in the art is familiar with how to detect markers in a sample. For example, commercial kits and/or well-developed methods are available for detection of creatinine (e.g., the “Creatinine Assay Kit” from Cell Biolabs, Inc.), free light chain ratio (see, e.g., Tosi, et al. Ther Adv Hematol 4:37-41 (2013)), M-spike (see, e.g., Noori, et al. Clinical Chemistry and Laboratory Medicine 59:1963-1971 (2021)), or hemoglobin (e.g., the “Hemoglobin Assay Kit” available from Millipore Sigma) in a sample.

Detection paradigms that can be employed in the disclosure include, but are not limited to, optical methods, electrochemical methods (e.g., voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Detection by Sequencing and/or Probes

In particular embodiments, the markers of the disclosure are analyzed by a sequencing- and/or probe-based technique (e.g., RNA-seq).

Any suitable method for isolation of DNA or RNA from the cells may be used in the methods of the disclosure (e.g., proteinase K-based purification methods). Various kits are commercially available for the purification of polynucleotides from a sample and are suitable for use in the methods of the disclosure (e.g., an ARCTURUS PICOPURE DNA Extraction Kit, Thermo Fisher Scientific). In an embodiment, the genomic DNA is purified using a proteinase K digestion-based technique (e.g., ARCTURUS PICOPURE DNA Extraction Kit, Thermo Fisher Scientific). In some embodiments, whole genome sequencing (WGS), such as deep WGS, is used. Sequencing methods may be used to detect sequence signatures such as somatic point mutations (e.g., single nucleotide variants (SNVs) and indels), copy-number alterations, translocations, and structural variants (e.g., non-coding mutations).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24;109(4):1347-52). After PCR, paired-end deep sequencing can be used to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample. Other transcriptomic methods may be suitable for use in the methods described herein.

Detecting a target polynucleotide sequence or fragment thereof associated with a marker that hybridizes to a probe sequence may involve sequencing, Fluorescence-Activated Cell Sorting (FACS), qPCR, RT-PCR, a genotyping array, and/or a NanoString assay, or any of various other techniques known to one of skill in the art.

Preparation of a library for sequencing typically involves an amplification step. Amplification may involve thermocycling or isothermal amplification (such as through the methods RPA or LAMP). Amplification may be used to add a known sequence, such as an adaptor, and/or an unknown sequence, such as a barcode, to a nucleic acid of interest. Amplification can refer to any method employing a primer and a polymerase capable of replicating a target sequence with reasonable fidelity. Amplification may be carried out by natural or recombinant DNA polymerases such as TAQGOLD, T7 DNA polymerase, Klenow fragment of E. coli DNA polymerase, and reverse transcriptase. One amplification method is PCR. In particular, the isolated RNA can be subjected to a reverse transcription assay that is coupled with a quantitative polymerase chain reaction (RT-PCR) in order to quantify the expression level of a marker.

Detection of the expression level of a marker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalator incorporated into the double-stranded DNA molecules is typically proportional to the amount of the amplified DNA products, one can determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TAQMAN probes) resulting in increased specificity and sensitivity. Methods for performing probe-based quantitative amplification are described, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated herein by reference).

In various embodiments, polynucleotides in a biological sample are sequenced using whole-genome sequencing and/or whole-exome sequencing. In some cases, it may be advantageous to sequence a sample to a high coverage (i.e., using “deep sequencing”). Deep sequencing is typically thought to provide more accurate sequencing results than lower-coverage methods.

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., ILLUMINA sequencing), sequencing with mass spectrometry, and tunneling currents DNA sequencing.

In embodiments, levels of markers in a sample are quantified using targeted sequencing. Methods for targeted sequencing are well known in the art (see, e.g., Rehm, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, 3-galactosidase, urease, alkaline phosphatase or peroxidase, avidin/biotin complex.

Methods used to detect or quantify binding of a probe to a target marker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 1251, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (CIBACRON Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the disclosure.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent barcode.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Immunoassay

In particular embodiments, the markers of the disclosure are measured by immunoassay. Immunoassay typically utilizes an antibody (or other agent that specifically binds the marker) to detect the presence or level of a marker in a sample. Antibodies can be produced by methods well known in the art, e.g., by immunizing animals with the markers. Markers can be isolated from samples based on their binding characteristics. Alternatively, if the amino acid sequence of a polypeptide marker is known, the polypeptide can be synthesized and used to generate antibodies by methods well known in the art.

This disclosure contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time, or a multiplex format may be used. Multiplex immunoanalysis may involve planar microarrays (protein chips) and bead-based microarrays (suspension arrays).

In a SELDI-based immunoassay, a biospecific capture reagent for the marker is attached to the surface of an MS probe, such as a pre-activated ProteinChip array. The marker is then specifically captured on the biochip through this reagent, and the captured marker is detected by mass spectrometry.

### Detection by Biochip

In embodiments, a sample is analyzed by means of a biochip (also known as a microarray). The polypeptides and nucleic acid molecules of the disclosure are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996). Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665.

### Detection by Protein Biochip

In embodiments, a sample is analyzed by means of a protein biochip (also known as a protein microarray). Such biochips are useful in high-throughput low-cost screens to identify alterations in the expression or post-translation modification of a marker, or a fragment thereof. In embodiments, a protein biochip of the disclosure binds a marker present in a sample and detects an alteration in the level of the marker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the disclosure) are spotted on a substrate using any convenient method known to the skilled artisan (e.g., by hand or by inkjet printer).

In embodiments, the protein biochip is hybridized with a detectable probe. Such probes can be polypeptides, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); tissue (e.g., bone marrow), a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In some embodiments of the disclosure, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT Publication No. WO95/251116 (Baldeschwieler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed, as described above.

The removal of nonhybridized probes may be accomplished, for example, by washing. The washing steps that follow hybridization can also vary in stringency, as described above.

Detection systems for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the markers of this disclosure are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in U.S. Pat. App. Pub. Nos: 20050023454; 20050035286; U.S. Pat. No. 5,800,979 and the references disclosed therein.

### Laser Desorption/Ionization

In embodiments, the mass spectrometer is a Laser Desorption/Ionization (LDI) mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of Matrix Associated Laser Desorption/Ionization (MALDI) or of Surface-Enhanced Laser Desorption/Ionization (SELDI).

LDI in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the disclosure is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead-based sample processing.

MALDI-TOF MS allows scanning of the fragments of many proteins at once. Thus, many proteins can be run simultaneously on a polyacrylamide gel, subjected to a method of the disclosure to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the disclosure. Fourier-transform ion cyclotron resonance (FTMS) can also be used for some embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in embodiments of the disclosure is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the markers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's PROTEINCHIP arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Pat. App. Pub. No. U.S. 2003/0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Pat. App. Pub. No. US 2003/0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the marker or markers that may be present in the sample to bind to the adsorbent. After an incubation period, the substrate is washed to remove unbound material. Any suitable washing solutions can be used, such as aqueous solutions. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound markers.

In yet another method, one can capture the markers with a solid phase bound immuno-adsorbent that has antibodies that bind the markers. After washing the adsorbent to remove unbound material, the markers are eluted from the solid phase and detected by applying to a SELDI biochip that binds the markers and analyzing by SELDI.

The markers bound to the substrates are detected in a gas phase ion spectrometer such as a time-of-flight mass spectrometer. The markers are ionized by an ionization source such as a laser, the generated ions are collected by an ion optic assembly, and then a mass analyzer disperses and analyzes the passing ions. The detector then translates information of the detected ions into mass-to-charge ratios. Detection of a marker typically will involve detection of signal intensity. Thus, both the quantity and mass of the marker can be determined.

Purification and/or Counting of Cancer Cells

The methods of the disclosure may involve characterizing the genomes of MM cells isolated from the peripheral blood or bone marrow of a subject. Such characterization may be facilitated by the isolation of circulating tumor cells from a sample (e.g., a liquid biopsy, such as a peripheral blood sample). In embodiments, genomic DNA from the circulating tumor cells is isolated and sequenced. In various embodiments, the MM cells are purified using an immunophenotype-based enrichment technique, such as Fluorescence-activated cell sorting (FACS) or CELLSEARCH.

In various embodiments, the methods of the disclosure involve sorting cells (e.g., circulating tumor cells, such as circulating multiple myeloma cells) obtained from a liquid biopsy (e.g., a blood sample) from a subject. The cells can be sorted and counted using any suitable method known in the art, such as an immunophenotype-based enrichment method. The cells can be sorted using a commercially available kit, such as the Silicon Biosystems Circulating Multiple Myeloma Cell Assay kit, which can be used in combination with a CELLSEARCH system. Non-limiting examples of immunophenotype-based enrichment methods include a CELLSEARCH system (an immunomagnetic and immunofluorescence imaging technology), and fluorescence activated cell sorting (FACS; e.g., high-sensitivity fluorescence-activated cell sorting). The immunophenotypes CD138+ and/or CD38+ can be used to select for plasma cells and the immunophenotypes CD45− and/or CD19− can be used to exclude non-PC leukocytes from a selection. In embodiments, a DAPI stain can be used to select for and/or detect nucleated cells.

In embodiments, the methods of the disclosure involve sequencing of DNA isolated from the enriched cells according to methods described herein. The sequence data obtained according to the methods of the disclosure allow for mutational analyses of iterating, clinically relevant and prognostic events of multiple myeloma including, as non-limiting examples, structural variation, copy number variation, and single nucleotide variation. In embodiments, the cells are enriched or isolated from a large background of mononuclear cells.

### Monitoring Hematological Malignancy Stage

Subjects being treated for a hematological malignancy (e.g., a monoclonal gammopathy) may be characterized using any of the methods described herein. Cells characteristic of a hematological malignancy typically display alterations in their genome compared to corresponding normal reference cells. Genetic alterations (e.g., mutations, chromosomal rearrangements, or aneuploidy) are correlated with multiple myeloma and related pathologies (e.g., MGUS, SMM).

In embodiments, the methods of the disclosure are used to monitor a patient. In some instances, monitoring of a patient involves characterizing a biological sample (e.g., a bone marrow biopsy, circulating tumor cells, or cell free DNA) from a subject according to the methods provided herein or at least about every 1 month, 6 months, 12 months, 18 months, or 24 months.

In these methods, a biological sample (e.g., a bone marrow biopsy or a liquid biopsy) is obtained from the subject characterized according to the methods provided herein. The biological sample can be, e.g., a body fluid such as blood or plasma, or a sample from a tumor from the subject. Typically, the biological sample is a blood sample (e.g., a peripheral blood (PB) sample). In various embodiments, the method involves assigning a MM-like score to the biological sample. The MM-like score is then used to inform the selection of a treatment for the subject.

### Subject Management

In certain embodiments, the methods of the disclosure involve managing subject treatment based on disease risk (e.g., low-risk, intermediate-risk, or high-risk) determined through measurement of an MM-like score according to the methods provided herein. Such management includes referral, for example, to a qualified specialist (e.g., an oncologist) and/or selecting a treatment strategy for the subject. In one embodiment, if a physician determines that a subject has an MM-like score associated with a low-risk, intermediate-risk, or high-risk MM, then a certain regime of treatment, such as prescription or administration of therapeutic agent might follow. Treatments that may be administered to the subject to treat a MM include, but are not limited to, chemotherapy, radiotherapy, immunotherapy, and surgery. Alternatively, a diagnosis of non-cancer might be followed with further testing to determine a specific disease that the patient might be suffering from or to determine whether a multiple myeloma in the subject has progressed (e.g., from one state in the development of a multiple myeloma to another, such as from MGUS to SMM or from SMM to MM). In some embodiments, subject management involves routine monitoring of multiple myeloma (MM) status in the subject through regular (e.g., weekly, monthly, yearly, etc.) characterization of biological samples (e.g., a bone marrow biopsy) from the subject.

Additional embodiments of the disclosure relate to the communication of assay results or diagnoses or both to technicians, physicians, or patients. In certain embodiments, computers will be used to communicate assay results or diagnoses or both to interested parties, e.g., physicians and their patients. In some embodiments, the assays will be performed, or the assay results analyzed in a country or jurisdiction which differs from the country or jurisdiction to which the results or diagnoses are communicated.

### Hardware and Software

The present disclosure also relates to a computer system involved in carrying out the methods of the disclosure relating to both computations and sequencing. The methods described herein, analyses can be performed on general-purpose or specially programmed hardware or software. One can then record the results (e.g., characterization of a CTC) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In some embodiments, the analysis is performed by an algorithm. The analysis of sequences will generate results that are subject to data processing. Data processing can be performed by the algorithm. One of ordinary skill can readily select and use the appropriate software and/or hardware to analyze a sequence. In some embodiments, an algorithm may be used to identify driver mutations, such as those identified in the Examples. An algorithm may be used to perform upstream analysis of raw sequencing data. Additionally, an algorithm may be used to calculate an MM-like score. In some embodiments, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In some embodiments, software used to analyze the data can include code that applies an algorithm to the analysis of the results. The software can also use input data (e.g., sequence) to characterize CTCs.

A computer system (or digital device) may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or processor on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine-readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high-capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in characterizing a biological sample from a subject. Kits of the instant disclosure may include one or more containers comprising an agent for enriching/isolating and/or characterization of a plasma cell sample and/or for treatment of a plasma cell dyscrasia, such as MM. In some embodiments, the kits further include instructions for use in accordance with the methods of this disclosure. In some embodiments, these instructions comprise a description of use of the agent to enrich/isolate and/or characterize a plasma cell sample and/or use of the agent for treatment of a plasma cell dyscrasia, such as MM. In some embodiments, the instructions comprise a description of how to isolate polynucleotides from a sample and/or to characterize a plasma cell sample. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed MYLAR or plastic bags), and the like. Kits may optionally provide additional components such as buffers and interpretive information. Normally, the kit comprises a container and a label or package insert(s) on or associated with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for specific embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### ILLUSTRATIVE EMBODIMENTS

Embodiment 1 is a method for selecting a subject having monoclonal gammopathy of undetermined significance (MGUS) or smoldering multiple myeloma (SMM) for administration of a therapeutic agent, the method including:
determining whether or not a biological sample from the subject contains a marker listed for each of categories a) to n) listed herein: a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2);
calculating an MM-like score for the subject, wherein the MM-like score is calculated by summing values assigned to each of categories a) to n), wherein each category is assigned a value of 0 if no markers in the category are detected, wherein each of categories a) and b) are assigned a value of −1 if a marker within the category is detected, and wherein each of categories c) to n) are assigned a value of +1 if a marker within the category is detected; and
selecting the subject for administration of the therapeutic agent if the MM-like score is equal to or greater than 4, wherein the subject is not selected for administration of the therapeutic agent if the MM-like score is less than 4.

Embodiment 2 is the method of any of the embodiments described herein, particularly embodiment 1, wherein the subject is selected for administration of the therapeutic agent if the MM-like score is greater than 5.

Embodiment 3 is the method of any of the embodiments described herein, particularly embodiment 1, wherein the subject is selected for administration of the therapeutic agent if the MM-like score is greater than 6.

Embodiment 4 is the method of any of the embodiments described herein, particularly embodiment 1, wherein the biological sample is a blood sample.

Embodiment 5 is the method of any of the embodiments described herein, particularly embodiment 1, wherein the biological sample is a bone marrow sample.

Embodiment 6 is the method of any of the embodiments described herein, particularly embodiment 1, wherein determining whether or not the biological sample from the subject contains a marker listed for each of categories a) to n) includes: analyzing sequencing data to identify the markers, wherein the sequencing data is produced by sequencing polynucleotides from the biological sample using whole-genome sequencing.

Embodiment 7 is the method of any of the embodiments described herein, particularly embodiment 6, wherein the whole-genome sequencing includes sequencing the polynucleotides to a coverage of at least 10-fold.

Embodiment 8 is the method of any of the embodiments described herein, particularly embodiment 7, wherein the whole-genome sequencing includes sequencing the polynucleotides to a coverage of at least 100-fold.

Embodiment 9 is the method of any of the embodiments described herein, particularly embodiments 6-8, wherein sequencing polynucleotides from the biological sample using whole-genome sequencing includes: enriching cancer cells from the biological sample and sequencing polynucleotides from the enriched cancer cells.

Embodiment 10 is the method of any of the embodiments described herein, particularly embodiments 6-8, wherein the polynucleotides include DNA.

Embodiment 11 is the method of any of the embodiments described herein, particularly embodiments 6-8, wherein the polynucleotides include RNA.

Embodiment 12 is the method of any of the embodiments described herein, particularly embodiment 1, wherein the therapeutic agent includes a BCMAxCD3 bispecific antibody.

Embodiment 13 is the method of any of the embodiments described herein, particularly embodiment 12, wherein the BCMAxCD3 bispecific antibody is selected from the group consisting of teclistamab, elranatamab, and linvoseltamab.

Embodiment 14 is the method of any of the embodiments described herein, particularly embodiment 1, wherein the therapeutic agent includes a GPRC5DxCD3 bispecific antibody. Embodiment 15 is the method of any of the embodiments described herein, particularly embodiment 14, wherein the GPRC5DxCD3 bispecific antibody is talquetamab.

Embodiment 16 is the method of any of the embodiments described herein, particularly embodiment 1, wherein the therapeutic agent includes a BCMA-directed CAR-T cell.

Embodiment 17 is the method of any of the embodiments described herein, particularly embodiment 16, wherein the BCMA-directed CAR-T cell is ciltacabtagene autoleucel or idecabtagene vicleucel.

Embodiment 18 is the method of any of the embodiments described herein, particularly embodiment 1, wherein the therapeutic agent includes an immunomodulatory agent and a glucocorticoid.

Embodiment 19 is the method of any of the embodiments described herein, particularly embodiment 18, wherein the immunomodulatory agent is lenalidomide and the glucocorticoid is dexamethasone.

Embodiment 20 is the method of any of the embodiments described herein, particularly 15 embodiment 1, wherein the therapeutic agent includes an anti-CD38 monoclonal antibody, an immunomodulatory agent, and a glucocorticoid.

Embodiment 21 is the method of any of the embodiments described herein, particularly embodiment 20, wherein the anti-CD38 monoclonal antibody is daratumumab or isatuximab, the immunomodulatory agent is lenalidomide, and the glucocorticoid is dexamethasone.

Embodiment 22 is a method for selecting a subject diagnosed as having multiple myeloma (MM) for administration of a therapeutic agent or autologous stem cell transplant for treating MM, the method including:
determining whether or not a biological sample from the subject contains a marker listed for each of categories a) to n): a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2);
calculating an MM-like score for the subject, wherein the MM-like score is calculated by summing values assigned to each of categories a) to n), wherein each category is assigned a value of 0 if no markers in the category are detected, wherein each of categories a) and b) are assigned a value of −1 if a marker within the category is detected, and wherein each of categories c) to n) are assigned a value of +1 if a marker within the category is detected; and
selecting the subject for administration of the therapeutic agent or autologous stem cell transplant if the MM-like score is equal to or greater than 2 and the subject is eligible for an autologous stem cell transplant, or selecting the subject for administration of the therapeutic agent if the subject is not eligible for an autologous stem cell transplant, and wherein the subject is not selected for administration of the therapeutic agent or autologous stem cell transplant if the MM-like score is less than 2.

Embodiment 23 is the method of any of the embodiments described herein, particularly embodiment 23, wherein the subject is eligible for an autologous stem cell transplant.

Embodiment 24 is the method of any of the embodiments described herein, particularly embodiment 23, wherein the MM-like score is equal to 2 or 3.

Embodiment 25 is the method of any of the embodiments described herein, particularly embodiment 24, wherein the therapeutic agent is a combination of the following: bortezomib; lenalidomide, thalidomide, or cyclophosphamide; and dexamethasone.

Embodiment 26 is the method of any of the embodiments described herein, particularly embodiment 25, wherein the method further includes administering to the subject lenalinomide as a maintenance treatment.

Embodiment 27 is the method of any of the embodiments described herein, particularly embodiments 22, 25, and 26, wherein the method includes administering the autologous stem cell transplant to the subject.

Embodiment 28 is the method of any of the embodiments described herein, particularly embodiment 23, wherein the MM-like score is greater than or equal to 4.

Embodiment 29 is the method of any of the embodiments described herein, particularly embodiment 28, wherein the therapeutic agent is a combination of the following: daratumumab; lenalidomide, iberdomide, or mezigdomide; bortezomib or carfilzomib; and dexamethasone.

Embodiment 30 is the method of any of the embodiments described herein, particularly embodiment 28, wherein the therapeutic agent includes a BCMAxCD3 bispecific antibody.

Embodiment 31 is the method of any of the embodiments described herein, particularly embodiment 30, wherein the BCMAxCD3 bispecific antibody is selected from the group consisting of teclistamab, elranatamab, and linvoseltamab.

Embodiment 32 is the method of any of the embodiments described herein, particularly embodiment 28, wherein the therapeutic agent includes a GPRC5DxCD3 bispecific antibody.

Embodiment 33 is the method of any of the embodiments described herein, particularly embodiment 32, wherein the GPRC5DxCD3 bispecific antibody is talquetamab.

Embodiment 34 is the method of any of the embodiments described herein, particularly embodiment 28, wherein the therapeutic agent includes a BCMA-directed CAR-T cell.

Embodiment 35 is the method of any of the embodiments described herein, particularly embodiment 34, wherein the BCMA-directed CAR-T cell is ciltacabtagene autoleucel or idecabtagene vicleucel.

Embodiment 36 is the method of any of the embodiments described herein, particularly embodiment 28, further including determining whether or not the biological sample contains the marker t(11;14), wherein if the biological sample contains the marker t(11;14), the therapeutic agent includes venetoclax.

Embodiment 37 is the method of any of the embodiments described herein, particularly embodiment 22, wherein the subject is not eligible for an autologous stem cell transplant.

Embodiment 38 is the method of any of the embodiments described herein, particularly embodiment 37, wherein the MM-like score is less than or equal to 3.

Embodiment 39 is the method of any of the embodiments described herein, particularly embodiment 38, wherein the therapeutic agent includes bortezomib, lenalidomide, and dexamethasone.

Embodiment 40 is the method of any of the embodiments described herein, particularly embodiment 38, wherein the therapeutic agent includes daratumumab, lenalidomide, and dexamethasone.

Embodiment 41 is the method of any of the embodiments described herein, particularly embodiments 39 and 40, wherein the method further includes administering to the subject lenalinomide as a maintenance treatment.

Embodiment 42 is the method of any of the embodiments described herein, particularly embodiment 37, wherein the MM-like score is greater than 4.

Embodiment 43 is the method of any of the embodiments described herein, particularly embodiment 42, wherein the therapeutic agent includes a combination of the following: a) bortezomib; b) lenalidomide, iberdomide, or mezigdomide; and c) dexamethasone.

Embodiment 44 is the method of any of the embodiments described herein, particularly embodiment 42, wherein the therapeutic agent includes a combination of the following: a) daratumumab; b) lenalidomide, iberdomide, or mezigdomide; and c) dexamethasone.

Embodiment 45 is the method of any of the embodiments described herein, particularly embodiments 43 and 44, wherein the method further includes administering to the subject lenalinomide and/or bortezomib as a maintenance treatment.

Embodiment 46 is the method of any of the embodiments described herein, particularly embodiment 42, wherein the therapeutic agent includes a BCMAxCD3 bispecific antibody.

Embodiment 47 is the method of any of the embodiments described herein, particularly embodiment 46, wherein the BCMAxCD3 bispecific antibody is selected from the group consisting of teclistamab, elranatamab, and linvoseltamab.

Embodiment 48 is the method of any of the embodiments described herein, particularly embodiment 42, wherein the therapeutic agent includes a GPRC5DxCD3 bispecific antibody.

Embodiment 49 is the method of any of the embodiments described herein, particularly embodiment 48, wherein the GPRC5DxCD3 bispecific antibody is talquetamab.

Embodiment 50 is the method of any of the embodiments described herein, particularly embodiment 42, wherein the therapeutic agent includes a BCMA-directed CAR-T cell.

Embodiment 51 is the method of any of the embodiments described herein, particularly embodiment 50, wherein the BCMA-directed CAR-T cell is ciltacabtagene autoleucel or idecabtagene vicleucel.

Embodiment 52 is the method of any of the embodiments described herein, particularly embodiment 42, wherein the therapeutic agent includes an immunomodulatory agent and a glucocorticoid.

Embodiment 53 is the method of any of the embodiments described herein, particularly embodiment 52, wherein the immunomodulatory agent is lenalidomide and the glucocorticoid is dexamethasone.

Embodiment 54 is the method of any of the embodiments described herein, particularly embodiment 42, wherein the therapeutic agent includes an anti-CD38 monoclonal antibody, an immunomodulatory agent, and a glucocorticoid.

Embodiment 55 is the method of any of the embodiments described herein, particularly embodiment 54, wherein the anti-CD38 monoclonal antibody is daratumumab or isatuximab, the immunomodulatory agent is lenalidomide, and the glucocorticoid is dexamethasone.

Embodiment 56 is the method of any of the embodiments described herein, particularly embodiment 42, further including:
determining whether or not the biological sample contains the marker t(11;14), wherein if the biological sample contains the marker t(11;14), the therapeutic agent includes venetoclax.

Embodiment 57 is the method of any of the embodiments described herein, particularly embodiment 22, wherein the biological sample is a blood sample.

Embodiment 58 is the method of any of the embodiments described herein, particularly embodiment 22, wherein the biological sample is a bone marrow sample.

Embodiment 59 is the method of any of the embodiments described herein, particularly embodiment 22, wherein determining whether or not the biological sample from the subject contains a marker listed for each of categories a) to n) includes:
analyzing sequencing data to identify the markers, wherein the sequencing data is produced by sequencing polynucleotides from the biological sample using whole-genome sequencing.

Embodiment 60 is the method of any of the embodiments described herein, particularly embodiment 59, wherein the whole-genome sequencing includes sequencing the polynucleotides to a coverage of at least 10-fold.

Embodiment 61 is the method of any of the embodiments described herein, particularly embodiment 60, wherein the whole-genome sequencing includes sequencing the polynucleotides to a coverage of at least 100-fold.

Embodiment 62 is a method for selecting a subject having monoclonal gammopathy of undetermined significance (MGUS), smoldering multiple myeloma (SMM) or multiple myeloma (MM) for inclusion in or exclusion from a clinical trial to study an agent for treatment of MM, the method including:
determining whether or not a biological sample from the subject contains a marker listed for each of categories a) to n): a) t(14;16)(MAF); b) t(14;20)(MAFB); c) a mutation in a KRAS gene; d) a mutation in an NRAS gene; e) a mutation in an FAM46C gene; f) hyperdiploidy or gain(3q26.2); g) del(1p), del(1p22.1), del(1p12), or del(1p32); h) gain(1q) or gain(1q21.2); i) del(4p16.3); j) del(4q34.3); k) del(8p) or del(8p23.3); 1) del(8q24.21), gain(8q24.21), or structural variation of MYC; m) del(16q), del(16q12.1); and n) del(17q21.2);
calculating an MM-like score for the subject, wherein the MM-like score is calculated by summing values assigned to each of categories a) to n), wherein each category is assigned a value of 0 if no markers in the category are detected, wherein each of categories a) and b) are assigned a value of −1 if a marker within the category is detected, and wherein each of categories c) to n) are assigned a value of +1 if a marker within the category is detected; and selecting the subject for inclusion in the clinical trial if the subject has MGUS or SMM and the MM-like score is greater than or equal to 4, and excluding the subject form the clinical trial if the subject has MGUS or SMM and the MM-like score is less than 4.

### EXAMPLES

### Example 1: Comprehensive Landscape of Somatic Mutations Across Precursor Multiple Myeloma (MM) and their Prognostic Value

To characterize the somatic mutation landscape and drivers in precursor conditions of MM, deep whole-genome sequencing (WGS) data was generated and analyzed on bone marrow (BM) samples from monoclonal gammopathy of undetermined significance (MGUS) (n=19), smoldering multiple myeloma (SMM) (n=119), and multiple myeloma (MM) (n=20) patients and their patient-matched germline controls. Since BM samples contain a small fraction of MGUS/SMM cells (median of 15%), these cells were enriched for and a low-input DNA protocol was used (Elis, et al., Nat Protoc. 16:841-71 (2021)) to reliably extract and sequence tumor DNA. This enabled the even characterization of patients from the three currently used SMM risk groups (IMWG 2/20/20 criteria: 44 low-risk, 29 intermediate-risk, and 46 high-risk SMM patients).

To increase cohort size and enable power for early driver discovery, the precursor-enriched data set was combined with 810 genomes and 853 exome profiles from 872 individuals with MGUS (n=18), SMM (n=62), and MM (n=792), yielding a MM and precursor map of somatic mutations from 1,030 individuals. For each tumor, somatic point mutations (single nucleotide variants (SNVs) and indels), copy-number alterations, and structural variants (non-coding mutations and SVs are for 969/1030 with WGS data; Methods) were identified. The analysis was well-powered to discover candidate drivers mutated in 2% of patients (Lawrence, et al. Nature, 505:495-501 (2014)). Indeed, down sampling analysis confirmed that the number of drivers found in 2% of patients plateaued at around 1,000 patients.

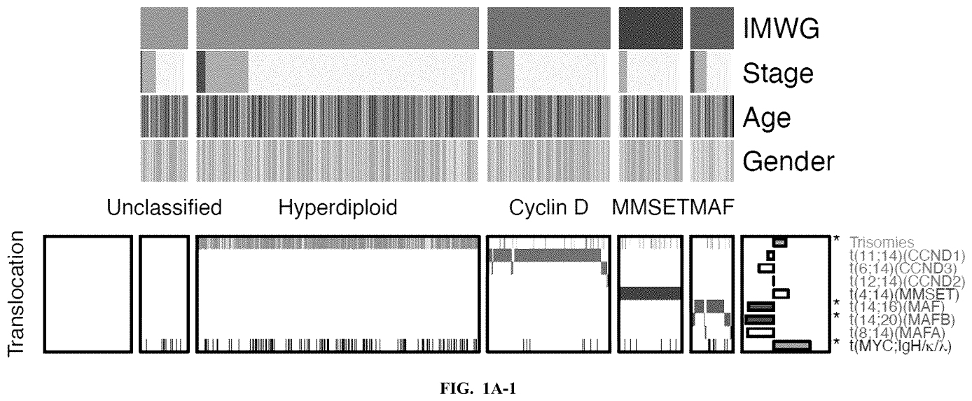

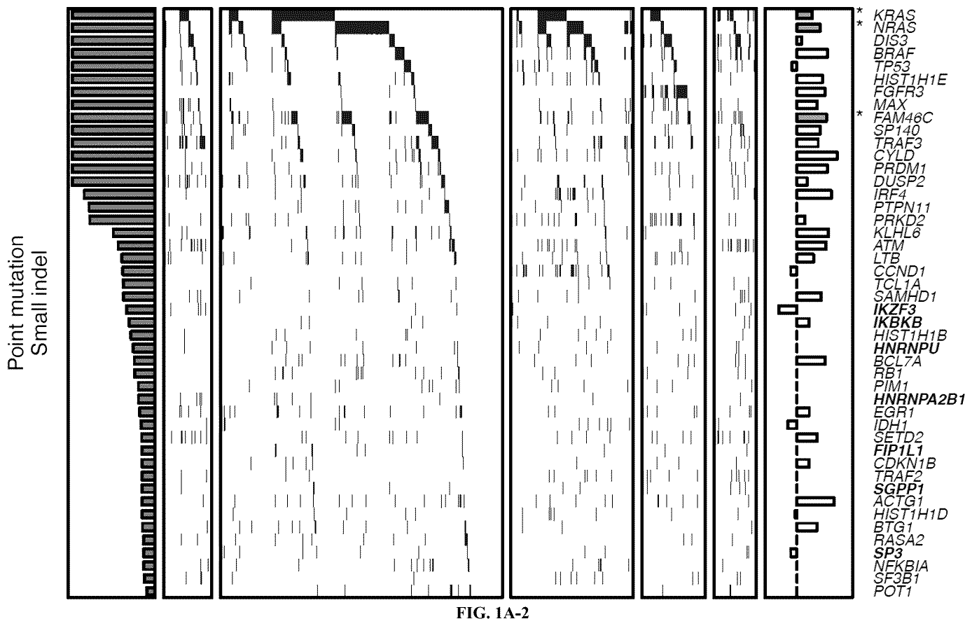

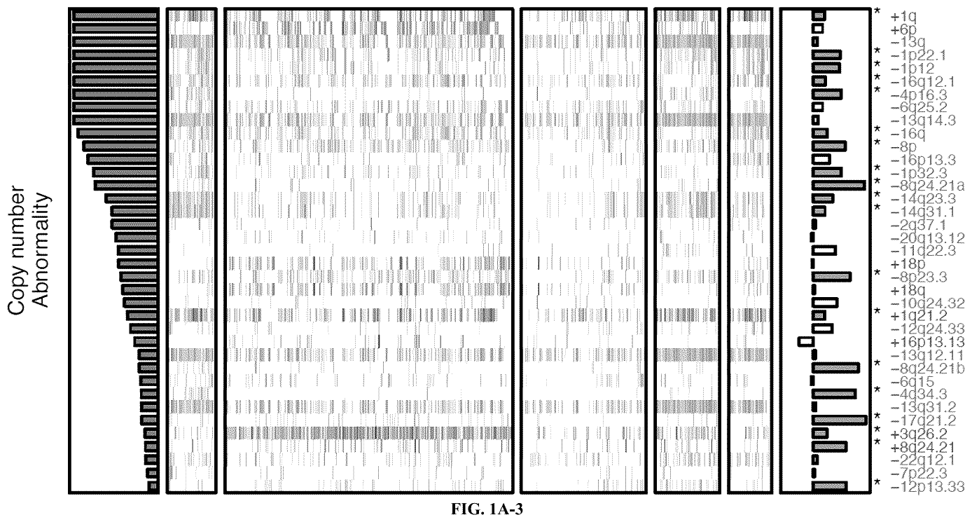

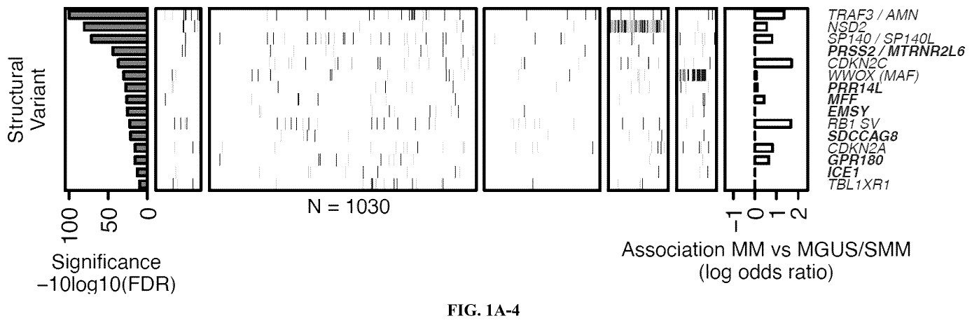

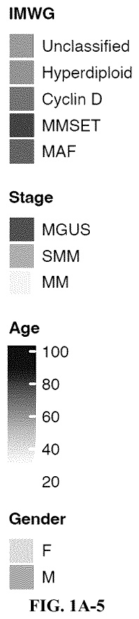

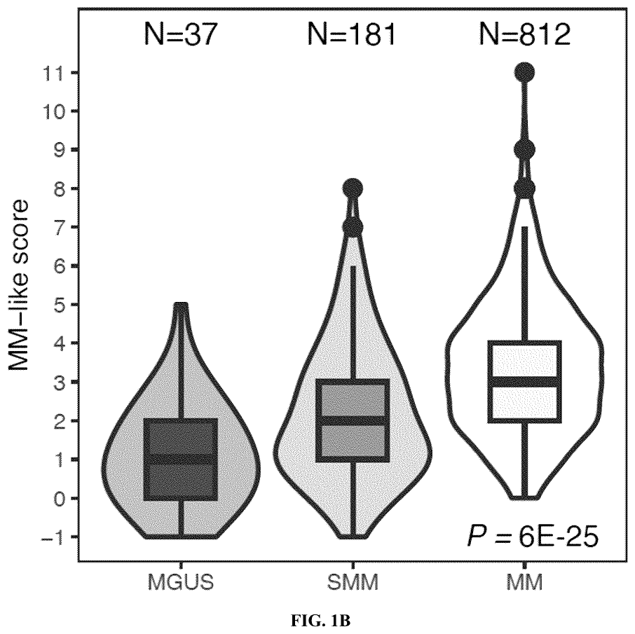

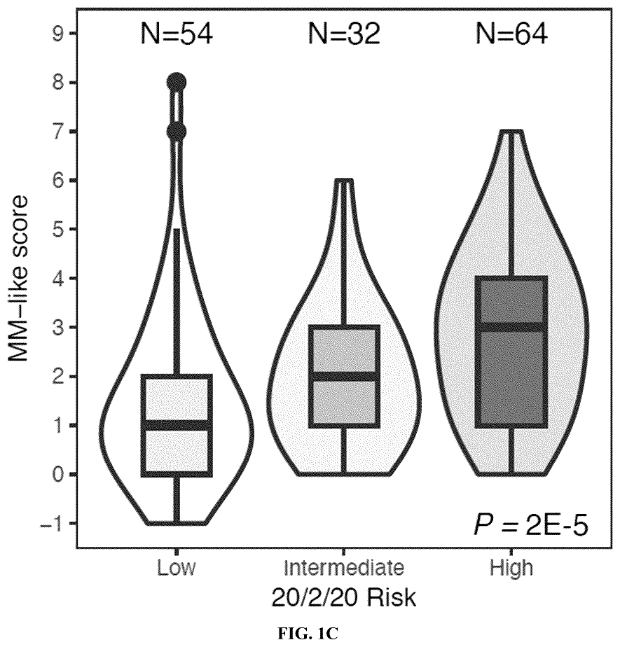

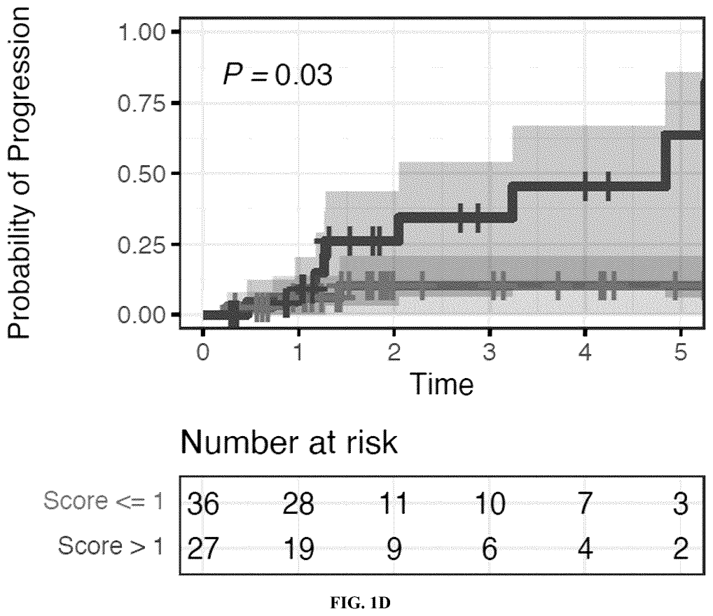

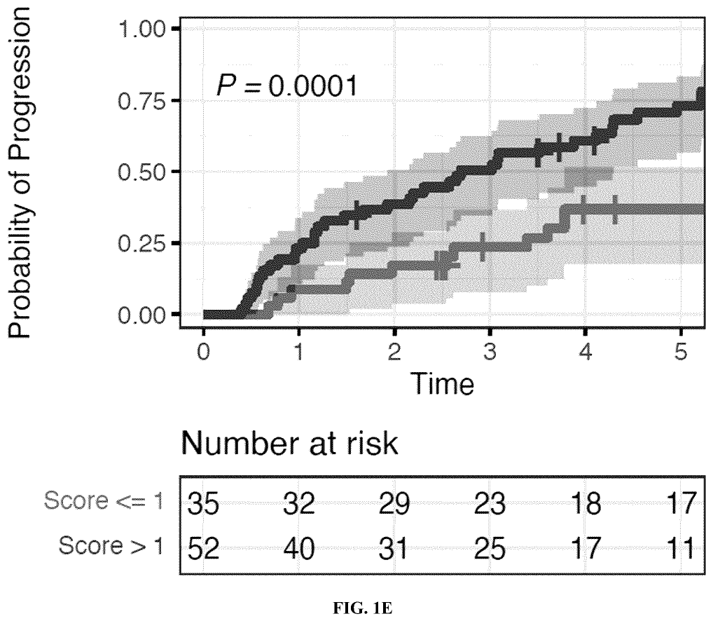

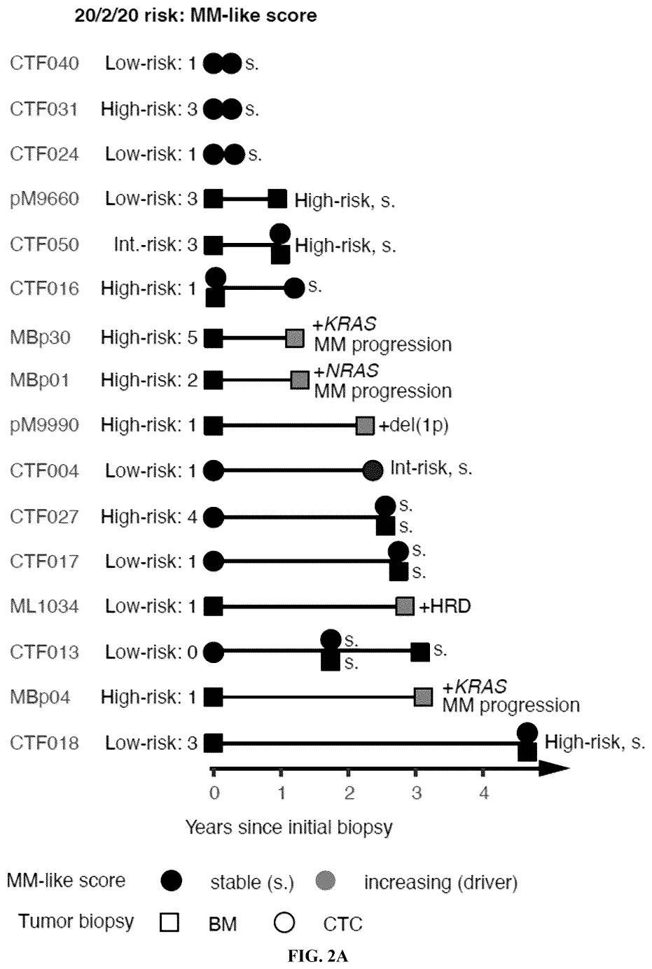

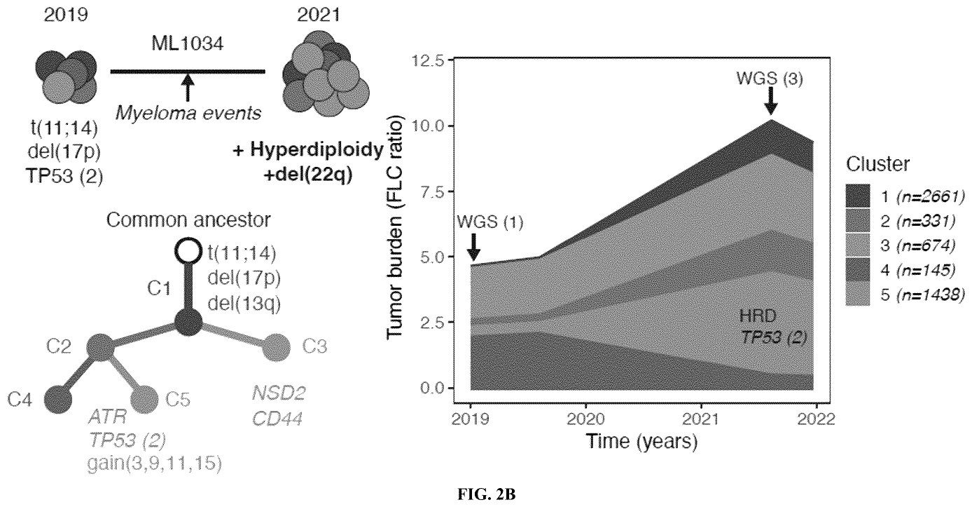

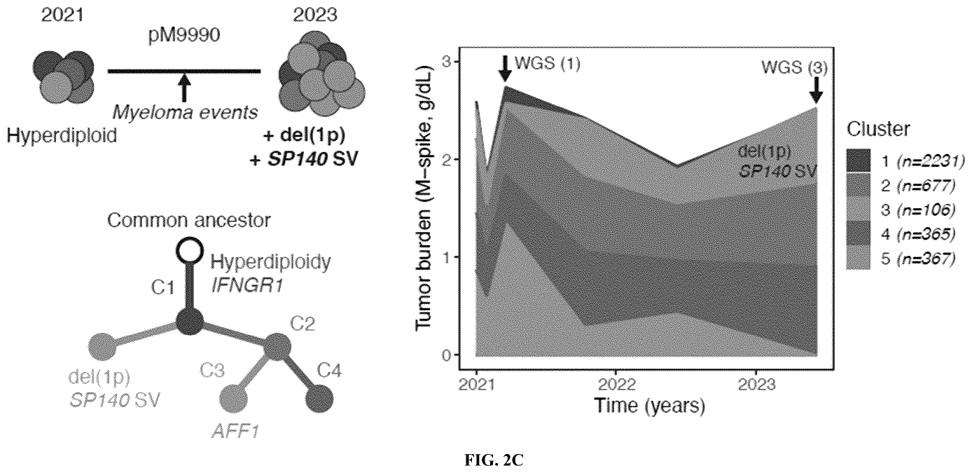

First, analyses were undertaken to discover candidate drivers of MM and precursor conditions. 62 genes with significantly recurrent point mutations (MutSig2CV q<0.1) were identified, including: (i) 39 already known MM drivers, such as KRAS, NRAS, FAM46C, DIS3, BRAF, and TP53; (ii) 7 new candidate drivers (IKZF3, IKBKB, HNRNPU, SP3, SGPP1, HNRNPA2B1 and FIPIL1); and (iii) 16 additional candidates mutated in <1% of patients (FIGS. 1A-1 to 1A-5). Notably, IKZF3 (mutated in 1% of patients) is required for the generation of high-affinity long-lived plasma cells and is also a known driver of chronic lymphocytic leukemia (CLL) and diffuse large B-cell lymphoma (DLBCL). For individuals where no MM SNV driver event was found, at least one canonical initiating MM event was evident, either one of known translocations, hyperdiploidy (trisomy of two or more of the odd chromosomes 3, 5, 7, 9, 11, 15, 19, 21), gain(1q) or del(13q). Next, copy number alterations were analyzed and 54 significant recurrently altered regions (21 chromosome arms: 18 gains and 3 losses; 33 focal events: 4 gains and 29 losses; GISTIC2.0 q<0.1) were found. Among these were the well-known hyperdiploidy gains, 1q, MYC, BCMA, FAM46C, CDKN2C, CYLD, and FGFR3 MAISET, as well as other significant regions including TERC GPRI60, EVI5, BCL6, BIRC2/3, and XBP1 (FIGS. 1A-1 to 1A-5). Finally, 15 additional candidate driver structural variations were discovered that included two known canonical drivers (FIGS. 1A-1 to 1A-5). When comparing the number of driver events across MM stages, it was observed that precursor patients already harbored at least one MM driver alteration (FIGS. 1A-1 to 1A-5). However, there was an increase in number of drivers with MM progression. For example, point mutation drivers were rare in MGUS (median of 0), increased in SMM (median of 1), and further increased in MM (median of 2) (FIG. 2A).

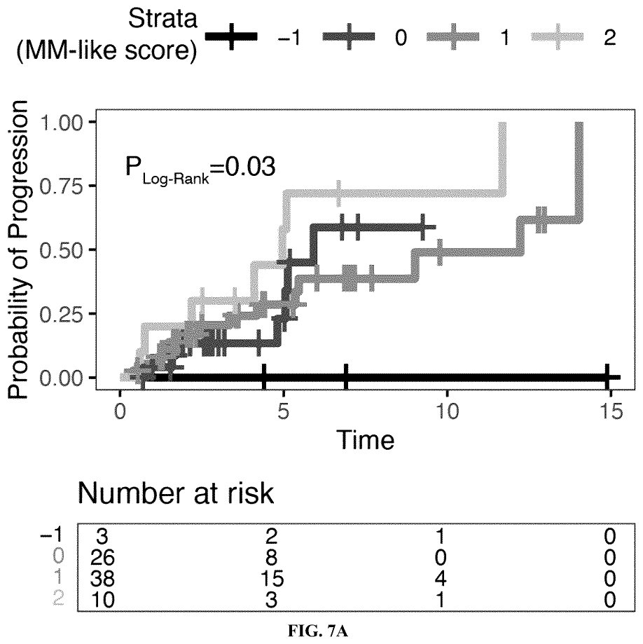

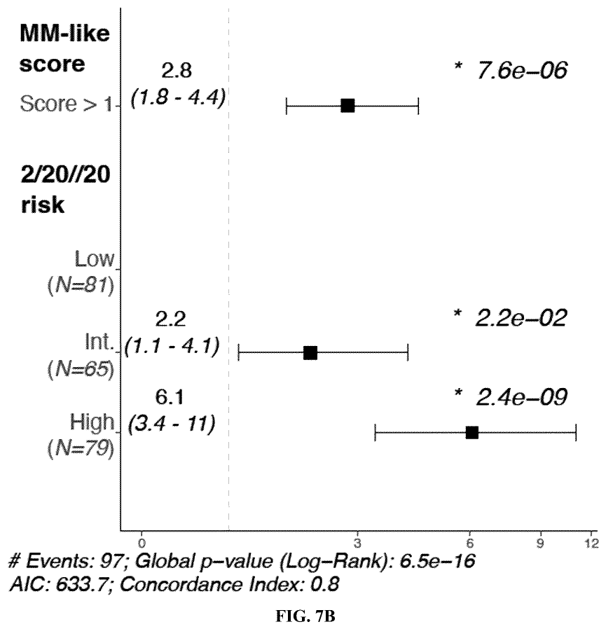

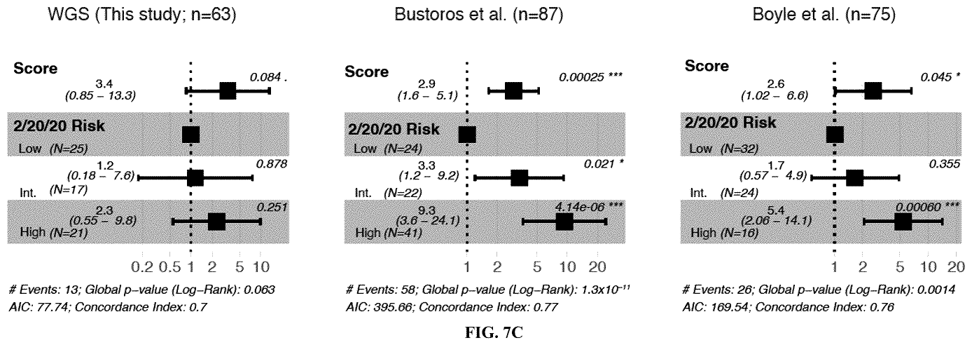

Next, work was undertaken to develop a simple score that reflects the similarity of precursor samples to overt MM and then test whether it can be used to estimate risk of progression. To do that, the 107 common driver events (occurring in >1% of patients) and initiating translocations were searched for ones that were differently altered in precursor conditions versus MM. Using the 812 WGS cases, 26 differentially altered events (q<0.1) were found. Only MAF and MAFB translocations were enriched in MGUS/SMM (MAF: OR=3.1; CI: 1.6-5.8; q=0.0026; MAFB: OR=3.4, CI:1.1-9.8, q=0.04), whereas 24 events were enriched in MM, including known MM drivers (MYC: OR=5.0, CI:2.2-14, q=5E-5; NRAS, OR=2.7, CI:1.5-5.3, q=0.009; KRAS, OR=1.9, CI:1.2-3.3, q=0.08; FIGS. 1A-1 to 1A-5). A simple “MM-like” score was then defined for each tumor as the number of MM-enriched drivers minus the number of MGUS-enriched drivers. The MM-like score in the cohort increased throughout MM stages of development (MM-like score median of 1 in MGUS, 2 in SMM and 3 in MM; FIG. 1B). Even among SMM patients, a gradual increase of the MM-like score was found from low- (median=1), to intermediate (median=2) to high-risk (median=3) groups that matched the overall medians in MGUS, SMM and MM, respectively (P=2×10⁻⁵; FIG. 1C). Among untreated SMM patients characterized with WGS, a higher MM-like score was associated with a higher likelihood of progression (Hazard ratio (HR)=5.3, CI_{95%}[1.1, 25], P=0.03; FIG. 1D). Using an independent dataset of 87 patients with SMM from Bustoros, et al., “Genomic Profiling of Smoldering Multiple Myeloma Identifies Patients at a High Risk of Disease Progression,” Journal of Clinical Oncology, 38:2380-90 (2020), doi: 10.1200/JCO.20.00437, it was validated that the MM-like score was indeed associated with a higher risk of progression (HR=3.3, CI_{95%}[1.8, 6.2], P=5×10-5; FIG. 1E). To provide additional validation of this association, an external validation dataset of 77 patients with SMM from Boyle et al., “The molecular make up of smoldering myeloma highlights the evolutionary pathways leading to multiple myeloma.” Nature Communications 12.1 (2021): 293.with deep targeted sequencing data, despite the fact that this dataset included only mutations and translocations (HR=1.8, CI_{95%}=[1.1-3.0], P=0.03; FIG. 7A). When analyzed in multivariate regression, the MM-like score was independently associated with a higher chance of progression in a model stratified by cohort (HR=2.8, CI_{95%}=[1.8-4.4], P=7.6E-6; FIG. 7B). The MM-like score independently correlated with progression in the two validation cohorts (Bustoros et al., and Boyle et al.). In the WGS cohort, the overall multivariate model was just above significance (P=0.06), and the MM-like score had the highest hazard ratio estimate compared to the 2/20/20 risk classification variables (FIG. 7C).

Overall, it was demonstrated that it was possible to stratify precursor stages of MM by discovering drivers, detecting those that were associated with the stage of the disease, and defining a genomically-based score that may be used to assess risk of progression, and is easy to implement in the clinic.

Table 3 below provides a list of new genes that were found to be drivers of MM such that mutations to these genes may be associated with the development of MM in a subject.

TABLE 3
List of new drivers.

| Gene | | |
|---|---|---|
| symbol | Long name | Comment |
| KRAS | v-Ki-ras2 Kirsten rat sarcoma viral | |
| | oncogene homolog | |
| NRAS | neuroblastoma RAS viral (v-ras) | |
| | oncogene homolog | |
| DIS3 | DIS3 mitotic control homolog (S. | |
| | cerevisiae) | |
| FAM46C | family with sequence similarity 46, | |
| | member C | |
| BRAF | v-raf murine sarcoma viral oncogene | |
| | homolog B1 | |
| TP53 | tumor protein p53 | |
| HIST1H1E | histone cluster 1, H1e | |
| FGFR3 | fibroblast growth factor receptor 3 | |
| | (achondroplasia, thanatophoric | |
| | dwarfism) | |
| SP140 | SP140 nuclear body protein | |
| MAX | MYC associated factor X | |
| TRAF3 | TNF receptor-associated factor 3 | |
| CYLD | cylindromatosis (turban tumor | |
| | syndrome) | |
| PRDM1 | PR domain containing 1, with ZNF | |
| | domain | |
| DUSP2 | dual specificity phosphatase 2 | |
| IGLL5 | immunoglobulin lambda-like | |
| | polypeptide 5 | |
| IRF4 | interferon regulatory factor 4 | |
| PTPN11 | protein tyrosine phosphatase, non- | |
| | receptor type 11 (Noonan syndrome 1) | |
| PRKD2 | protein kinase D2 | |
| KLHL6 | kelch-like 6 (Drosophila) | |
| ATM | ataxia telangiectasia mutated | |
| LTB | lymphotoxin beta (TNF superfamily, | |
| | member 3) | |
| CCND1 | cyclin D1 | |
| TCL1A | T-cell leukemia/lymphoma 1A | |
| SAMHD1 | SAM domain and HD domain 1 | |
| IKZF3 | IKAROS family zinc finger 3 (Aiolos) | New candidate driver; overlaps |
| | | with copy-number abnormalities |
| | | Del(17q21.2) |
| IKBKB | inhibitor of kappa light polypeptide | New candidate driver |
| | gene enhancer in B-cells, kinase beta | |
| HIST1H1B | histone cluster 1, H1b | |
| HNRNPU | heterogeneous nuclear | New candidate driver |
| | ribonucleoprotein U (scaffold | |
| | attachment factor A) | |
| DDX5 | DEAD (Asp-Glu-Ala-Asp) box | Rare (<1%) |
| | polypeptide 5 | |
| SMU1 | smu-1 suppressor of mec-8 and unc-52 | Rare (<1%) |
| | homolog (C. elegans) | |
| BCL7A | B-cell CLL/lymphoma 7A | |
| RB1 | retinoblastoma 1 (including | |
| | osteosarcoma) | |
| RNASEH2C | ribonuclease H2, subunit C | Rare (<1%) |
| PIM1 | pim-1 oncogene | |
| HNRNPA2B1 | heterogeneous nuclear | New candidate driver |
| | ribonucleoprotein A2/B1 | |
| RPL5 | ribosomal protein L5 | Rare (<1%) |
| EGR1 | early growth response 1 | |
| IDH1 | isocitrate dehydrogenase 1 (NADP+), | |
| | soluble | |
| ATP5D | ATP synthase, H+ transporting, | Rare (<1%) |
| | mitochondrial F1 complex, delta subunit | |
| SETD2 | SET domain containing 2 | |
| FRMPD2 | FERM and PDZ domain containing 2 | Rare (<1%) |
| FIP1L1 | FIP1 like 1 (S. cerevisiae) | New candidate driver |
| CDKN1B | cyclin-dependent kinase inhibitor 1B | |
| | (p27, Kip1) | |
| EEF1A1 | eukaryotic translation elongation factor | Rare (<1%) |
| | 1 alpha 1 | |
| TRAF2 | TNF receptor-associated factor 2 | |
| SGPP1 | sphingosine-1-phosphate phosphatase 1 | New candidate driver |
| FAM196A | family with sequence similarity 196, | Rare (<1%) |
| | member A | |
| IRF1 | interferon regulatory factor 1 | Rare (<1%) |
| ACTG1 | actin, gamma 1 | |
| TGDS | TDP-glucose 4,6-dehydratase | Rare (<1%) |
| HIST1H1D | histone cluster 1, H1d | |
| HIST1H4E | histone cluster 1, H4e | Rare (<1%) |
| BTG1 | B-cell translocation gene 1, anti- | |
| | proliferative | |
| ANKRD9 | ankyrin repeat domain 9 | Rare (<1%) |
| HOOK1 | hook homolog 1 (Drosophila) | Rare (<1%) |
| RASA2 | RAS p21 protein activator 2 | |
| SP3 | Sp3 transcription factor | New candidate driver |
| NFKBIA | nuclear factor of kappa light | |
| | polypeptide gene enhancer in B-cells | |
| | inhibitor, alpha | |
| EFTUD2 | elongation factor Tu GTP binding | Rare (<1%) |
| | domain containing 2 | |
| KRT2 | keratin 2 (epidermal ichthyosis bullosa | Rare (<1%) |
| | of Siemens) | |
| SF3B1 | splicing factor 3b, subunit 1, 155 kDa | |
| FKBP14 | FK506 binding protein 14, 22 kDa | Rare (<1%) |
| POT1 | POT1 protection of telomeres 1 | |
| | homolog (S. pombe) | |
| MCPH1 | Microcephalin | Found in deletion(8p) (no point |
| | | mutation) |
| CASP3 | Caspase-3 | Found in deletion(4q34.3) (no point |
| | | mutation) |

### Example 2: The “MM-Like” Score Captured Tumor Evolution in Serially Sampled SMM Patients

It was assessed whether the “MIM-like” score of serial samples from SMM patients reflected their clinical course. To that end, 39 serial tumor samples (BM or circulating tumor cells) were collected from 16 patients, taken at various time points (from diagnosis to progression) over a period of up to 54 months. The MNM-like score was calculated for each sample based on whole-genome sequencing (WGS) data and associated with the standard tumor burden measurements and clinical progression. It was observed that the dynamic spectrum of the disease was indeed captured by the MNM-like score (FIG. 2A). For 11 patients who did not progress or increased their tumor burden, the MMN-like score was stable over time. These included 6 patients with a follow up time that was less that the first time to progression (14 months) and likely did not have sufficient time to acquire an additional driver event and expand that subclone to a detectable level. Three patients clinically progressed, and their MM-like score increased at the time of progression.

The two remaining patients increased their MM-like score; however, they did not clinically progress (during the measured intervals), but one of them doubled their free light chain ratio, suggesting advancement towards progression. The increases in the MM-like score were due to different driver events, including acquiring KRAS (G13D) in two progressors, NRAS (G13D) in the third progressor, and del(1p), and hyperdiploidy in two non-progressors, respectively.

To better understand the evolution of the tumor in the two patients (Pts. ML1034 and pM9990) that increased their score but not yet progressed, the subclonal structure of the tumors and their phylogenetic trees were analyzed. Patient ML1034 (FIG. 2B) represented an interesting case of light chain MGUS/Low risk smoldering with a follow-up sample collected after three years. At baseline, a canonical t(11;14) translocation, a clonal del(17p)(TP53) and two subclonal missense mutations in TP53 (p.S241F, CCF: 24%; p.Y205D, CCF: 28%) were detected. After three years, an increase was observed in both tumor burden (free light chain ratio: 4.6 to 9.4) and CCF of the TP53 mutations (CCF: 50% and 36%, respectively), as well as a newly detected subclonal hyperdiploidy (gains of chr3, 9, 11, and 15; FIG. 2B). The phylogenetic structure of these samples was analyzed and hyperdiploidy was identified in the follow-up sample in cluster 5 (FIG. 2B), yielding an increasing MM-like score from 1 to 2. In contrast, patient pM9990 was initially characterized by hyperdiploidy with no other MM driver event (MM-like score of 1; FIG. 2C), but in the follow-up sample, del(1p) was identified, increasing the MM-like score to 2. Phylogenetic analysis (using PhylogicNDT) identified an increasing subclone with the del(1p) (C5 in FIG. 2C) and other subclones in a separate part of the tree that either shrank on were stable over time (C2, C3 and C4 in FIG. 2C). Taken together, these data show the ability of WGS data and the MM-like score to successfully capture the acquisition of new MM drivers and, using phylogeny dynamics, demonstrate the growth of the subclone that acquired the additional driver.

### Example 3: Inferred Timing of Mutations and Evolutionary Trajectory of Precursor Multiple Myeloma (MM)

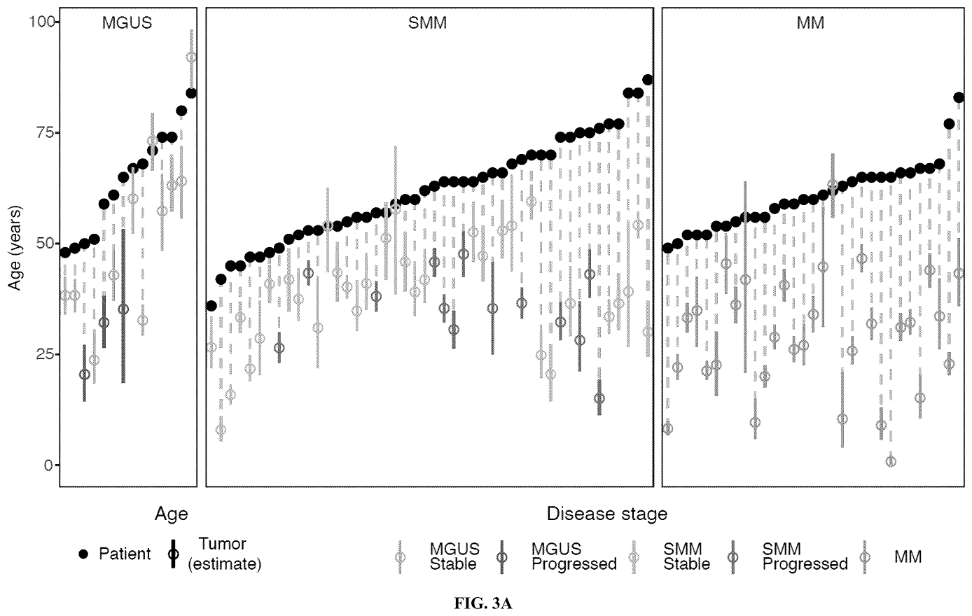

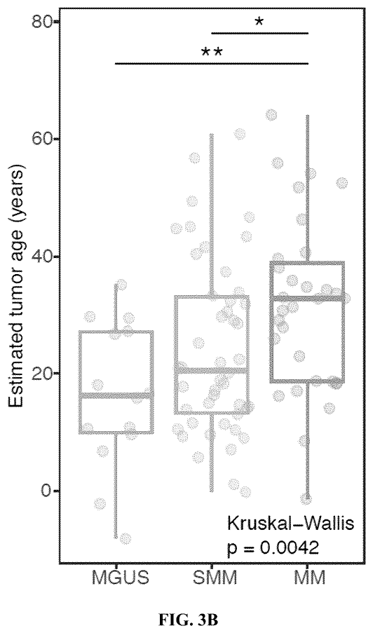

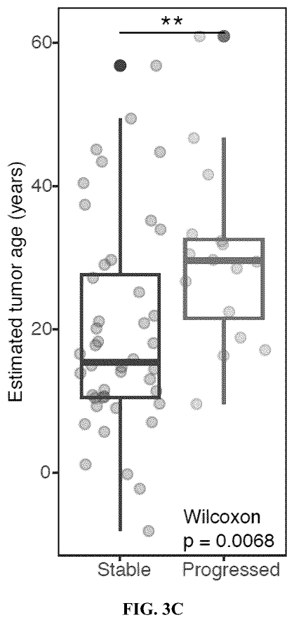

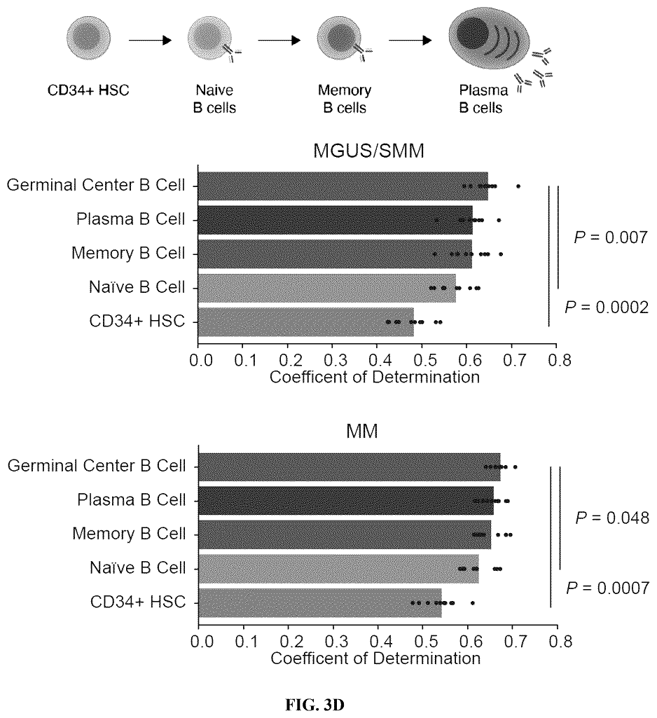

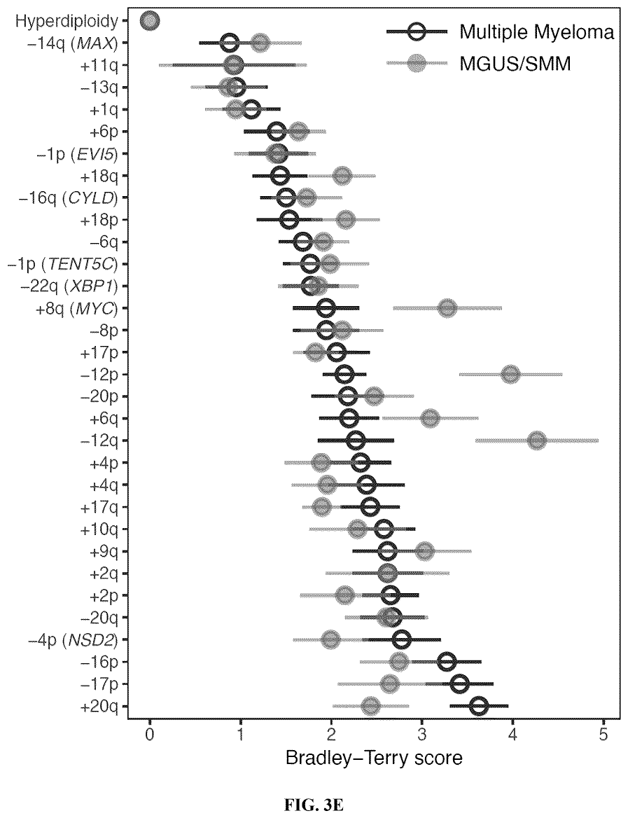

The time of initiation and cell of origin for MM precursor disease are widely debated. To further delineate the origins of the disease, the time of the onset of the disease was estimated based on passenger mutations that accumulate in a clock-like manner. In particular, the 91 deep WGS tumors (14 MGUS, 46 SMM, and 31 MM) that had a clonal hyperdiploidy event were focused upon, where the clonal hyperploidy event was assumed to be the initiating event. By comparing the number of duplicated and non-duplicated mutations (from clock-like mutational signatures SBS1 and SBS5) in the amplified chromosomes, the fraction of time from the zygote to acquiring the hyperdiploidy events could be estimated. Then, this fraction (and its confidence internal) could be converted to actual age at initiation, by multiplying it by the patient's age at the time of biopsy (FIG. 3A). There was a wide range of estimated ages of initiation, from as low as 1 year old up to 92 years old (median of 35). Interestingly, it was found that the patient's age at tumor initiation (i.e., hyperdiploidy) correlated with the stage of the disease; the more advanced stages were initiated at younger age (MGUS: 41 years old, SMM: 38 years old, MM: 31 years old, P=0.0035, FIG. 3B). It was also possible to estimate the age of each tumor by subtracting the tumor initiating age from the patient's age at the time of biopsy, and it was confirmed that MGUS and SMM were younger tumors than MM (MGUS: 16 years old, SMM: 21 years old, MM: 33 years old; P=0.004; FIG. 3C).

Next, in patients with MGUS or SMM, it was endeavored to test whether the tumor's age was associated with progression to MM. Indeed, it was found that progressors had a significantly older tumor than patients with a stable disease (median: 15 years old tumor in stable disease, versus 30 years old in progressors; P=0.007; FIG. 3D). Together, it was shown that WGS-based molecular clocks could be leveraged to differentiate disease severity and risk of progression, and that the earliest oncogenic events of MM could occur in the first decades of life.

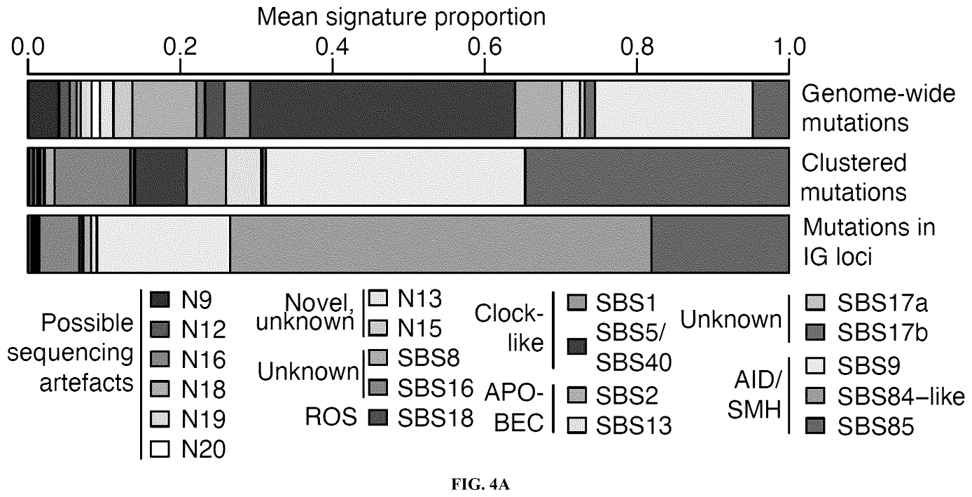

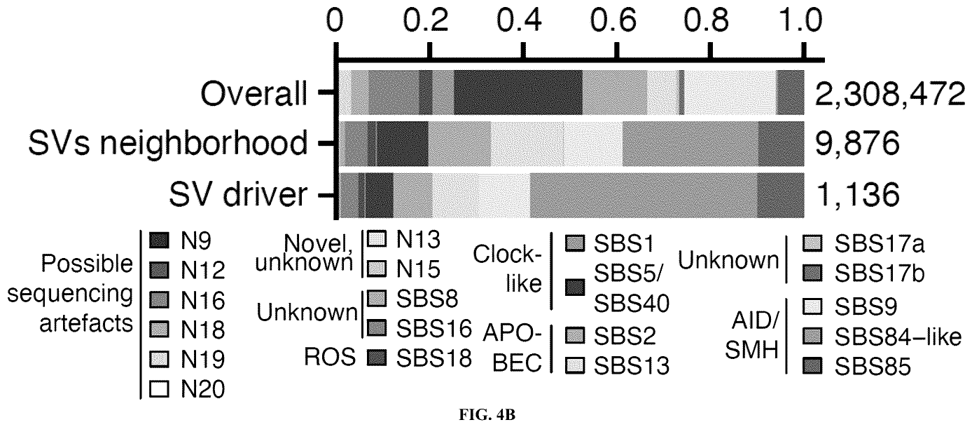

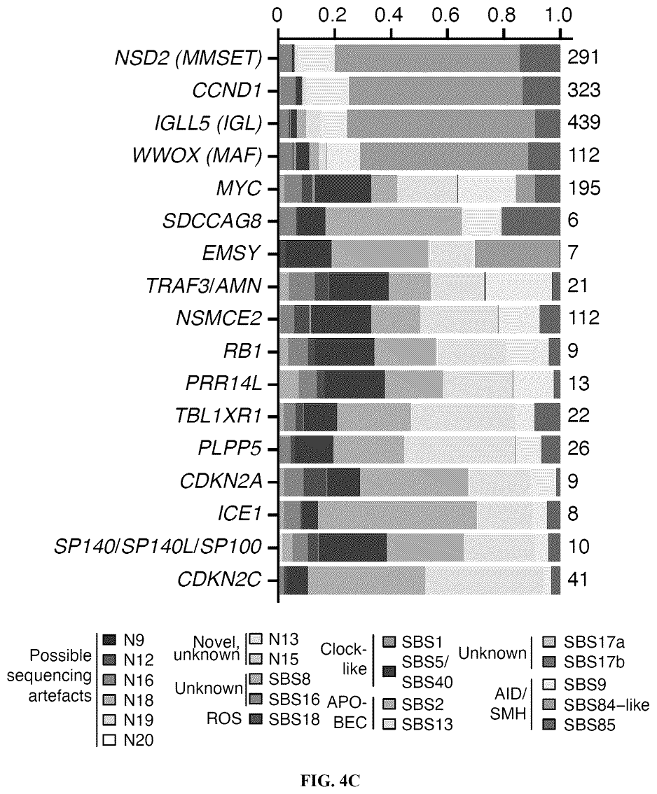

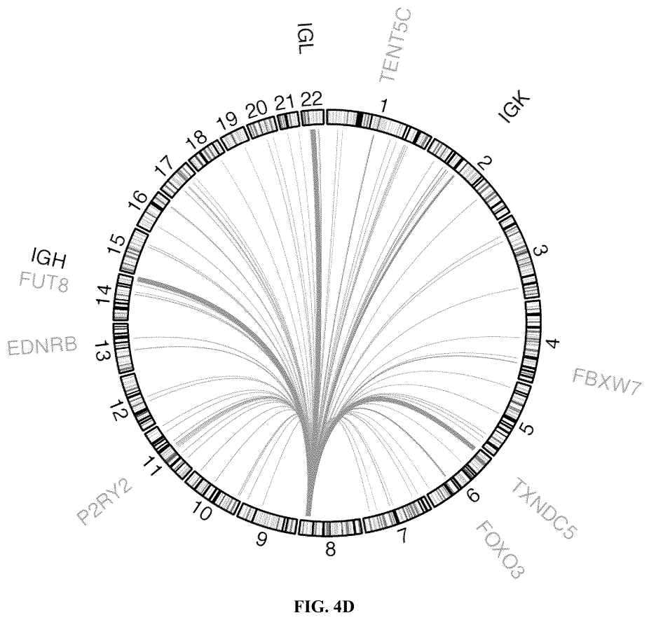

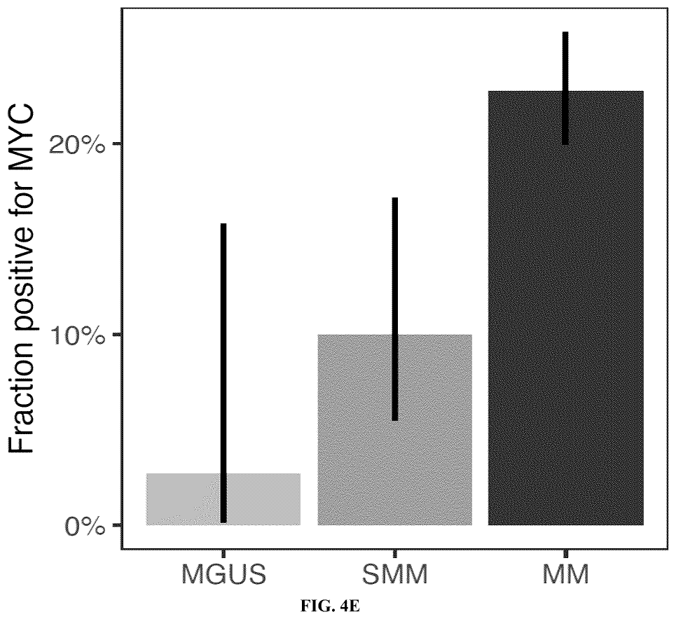

The local distribution of mutations is shaped by epigenetic organization, which varies along a single genome as well as between cell types. This correlation was made use of to further elucidate the B cell differentiation stage at which MGUS/SMM acquired most of its mutations. To this end, the Cell-of-Origin (COO) prediction method was used, which leverages correlations between somatic mutation profiles and epigenetic tracks from healthy tissues (Kübler, et al., “Tumor mutational landscape is a record of the pre-malignant state,” bioRxiv. 2019:517565. doi: 10.1101/517565; and Machado, et al. “Diverse mutational landscapes in human lymphocytes,” Nature, 608:742-32 (2022)). It was found that mature B cells were the healthy cell type with the highest correlation to the mutational landscape of MGUS/SMM. Germinal center (GC) B cells performed best (coefficient of determination [CoD]=0.65), but statistical significance was only reached for comparisons involving early immature B cell types (naïve B cells: P=0.007, HSCs: P=0.0002; corrected t-test), suggesting that most passenger mutations were acquired during the later stages of B cell differentiation, including memory B cells and PCs (FIG. 3D). Of note, the COO predictions were consistent across MM subgroups (all CoD >0.5) and stages (all CoD >0.6) and showed somatic mutation profiles systematically correlate with the epigenetic landscape of mature B cell types. Next, the MM COO analysis was compared with tumor genomic data from non-Hodgkin lymphoma (NHL) and chronic lymphocytic leukemia (CLL) from the International Cancer Genome Consortium (ICGC). It was found that NHL also correlated best with GC B cells (CoD=0.7; P=0.047). In contrast, CLL had comparable correlations with memory B cells (CoD=0.7) and GC B cells (CoD=0.69) as top hits (P=0.02; Extended Data FIG. 4). Overall, without intending to be bound by theory, this suggested that most mutations in these three B cell receptor (BCR)-rearranged neoplasias were acquired during mature B cell differentiation stages, and that only MM involves additional mutation acquired in plasma cells (PCs).

To estimate the relative order of the acquisition of MM driver events from the MGUS/SMM stages, a Bradley-Terry (BT) model was developed based on the clonality levels of MM copy-number drivers that was termed Clonality League Competition (CLC). In this model, each MM driver event was given a score (BT score) that reflected the probability that such an event was found in smaller subclones compared to others. This allowed for the ranking of MM driver events: those that were most often clonal had the lowest BT score, whereas events, which are thought to happen late in cancer development, had higher BT scores. Then, to test whether the order of acquisition of events differed between MGUS/SMM and MM stages, the distributions of BT scores between both models was compared. Overall, a positive correlation of BT scores between MGUS/SMM and MM (R=0.61, P=1.1×10⁵) was found, suggesting the overall sequence of acquisition of driver events was comparable across stages (FIG. 3D). Indeed, in both league competitions, events which are known to happen early had the lowest scores (Hyperdiploidy, del(14q), gain(11q), del(13q), and gain(1q); BT scores <1.4: FIG. 3D). In contrast, deletion 17p (encompassing TP53), which is known to happen late and remain subclonal during disease development, had an elevated score in both leagues (MGUS/SMM: 3.4, MM: 2.6; P=3.3×10⁻⁴), validating that the model captured known patterns of MM oncogenesis.

Next, a search was made for events that would mark the transformation from MGUS/SMM to MM. To identify these events, events were identified which had higher BT scores in the MGUS/SMM league than in the MM one. 20 such events were found: 3 were strongly enriched in MM (more clonal in MM: gain(8q24)(MYC), del(12p), and del(12q)) while the remaining 17 had a difference in BT-score (<1). Two of the events that happened early in MM, but late in MGUS/SMM (MYC and del(12p)) were also identified by the MM-like score, while deletion 12q may be correlated to 12p in cases with whole chromosome deletion. Overall, the Bradley-Terry model further supported the role ofMYC abnormalities in the transformation from MGUS/SMM to MM and proposed that del(12) bearing GPRC5D provided similar proliferation advantage to become clonal at the time of overt disease diagnosis.

### Example 4: Mutational Processes and Structural Variants that Shape Monoclonal Gammopathy of Undetermined Significance/Smoldering Multiple Myeloma (MGUS/SMM)

Mutational processes active before and during MM oncogenesis have been extensively described; however, little is known how accumulating passenger events leads to mutating MM drivers. To map MM 11 drivers and mutational processes, the hdp algorithm was used to identify mutational signatures in WGS data. 19 signatures were identified, of which 12 were already known and made up a fraction of the total mutational burden, which was annotated with the COSMIC catalogue of signatures: somatic hypermutation and activation-induced cytidine deaminase (SHM/AID): SBS9, SBS84, SBS85; APOBEC: SBS2, SBS13; clock-like: SBS1, SBS5/40; reactive oxygen species (ROS) signature SBS18 and signatures of unknown etiology SBS8, SBS16, SBS17a, SBS17b; FIG. 4A). 7 signatures were also detected which were not in the COSMIC catalogue, of which 5 were possible technical artefacts and 2 were annotated as novel, for which a clear etiology was not identified. In line with data from CLL (Kasar, et al., “Whole-genome sequencing reveals activation-induced cytidine deaminase signatures during indolent chronic lymphocytic leukaemia evolution,” Nature Communications, 6:8866 (2015), doi: 10.1038/ncomms9866), it was found that three signatures associated with SHM/AID activity (SBS9, SBS84, and SBS85) were highly enriched in clustered mutations (<1,000 base pairs from each other; FIG. 4A), and the association was even stronger when the immunoglobulin loci were analyzed, which are targets of physiological SHM (FIG. 4A, bottom row). Those mutations associated with AID/SHM and clock-like signatures were mostly clonal (median CCF probability >0.85(cite Landau)). On the contrary, mutations that were associated with APOBEC activity were mostly found in subclones (median CCF <0.85), suggesting APOBEC activity happens later and is still ongoing in many patients. To further clarify this point, the relation between the frequency of mutational signatures in patients and with disease stages and subgroups of the disease was modeled. It was found that molecular subgroups (translocations, hyperdiploidy, etc.), but not disease stages (MGUS/SMM and MM), were associated with mutational signatures. Specifically, it was confirmed that APOBEC signatures SBS2 and SBS3 were increased in the MAF subgroup of patients, and novel positive associations between the ROS signature (SBS18) and the Hyperdiploid subgroup of patients, as well as between the SBS8 of unknown etiology and the Cyclin D subgroup were found. Then, the fraction of clonal mutations that were attributed to APOBEC in patients from the MAF subgroup versus non-MAF was calculated and compared with other mutational signatures. It was found that APOBEC mutations were mostly subclonal in the majority of patients who were not from the MAF subgroup, suggesting that in those patients specifically, APOBEC was a late and ongoing mutational process. On the contrary, other subgroups displayed similar rates of clonal mutations across putative mutational processes.

It was then determined whether local AID mutational signature could be leveraged to identify the cause of structural variant (SV) drivers, which were expected to happen for initiating translocations that were caused by an AID-related double strand break (DSB) repaired in trans. In order to discover novel SV drivers in addition to known initiating events, a method called SVGAR-sf was developed, which, for each gene, analyzed structural variants in their neighborhood and estimated whether SVs were statistically enriched for causing loss-of-function events. The effect of SV hotspots was identified in 659 patients (68%) across all stages of MM, including but not limited to a recurrent subgroup defining immunoglobulin heavy locus (IGH) initiating translocations (NSD2/MMSET in t(4;14), WWOX proximal to MAF in t(14;16); FIGS. 1A-1 to 1A-5). In addition to two common IGH translocations, the method detected 13 candidates that were found significant in MGUS/SMM and MM samples. This list included 6 known drivers (CDKN2A, CDKN2C, SP140, RB1, TBLIXR1, and TRAF3) as well as 7 novel candidates (EMSY, GPR180, ICE1, MFF, PRR14L, PRSS2, and SDCCAG8). With the exception of IGH translocations, SV driver mutations were less common in MGUS/SMM genomes (8%, CI: 4-13%) than in MM (27%, CI: 24-30%, P=4E-6). It was found that the close neighborhood of SVs overall was enriched in AID signature (<10,000 base pairs; FIG. 4B); and the association was even stronger among SV drivers alone. Within SV drivers, two categories could be differentiated: IGH translocations (MMSET, CCND, MAF) and IGL (IGLL5) were highly enriched in AID, suggesting they arose from in trans DSB repairs caused by AID during cell differentiation. On the contrary, all 13 other SV drivers were marked by APOBEC signatures (SBS2 and SBS13), which is recurrent across cancers and not part of the physiological B cell development. Together, this shows that the analysis of WGS data is able to detect new MM candidate drivers from the genomic location of structural variants and can associate them with mutational processes that were likely involved in those structural variants.

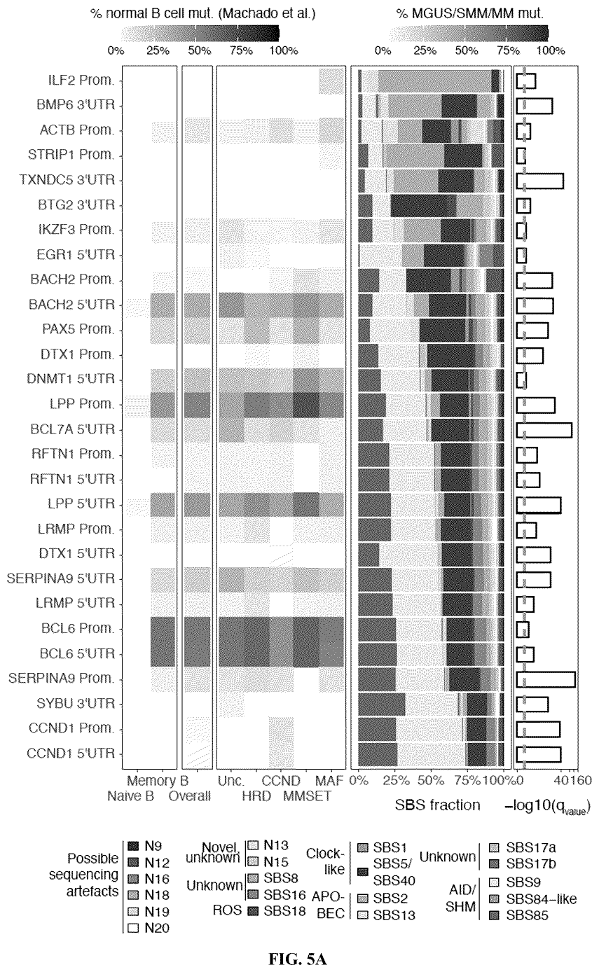

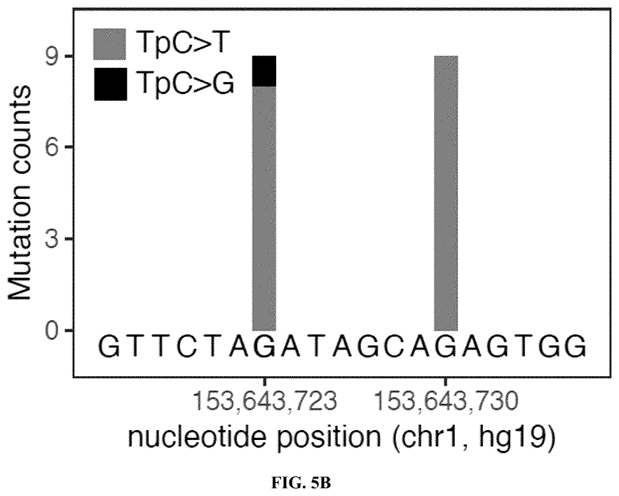

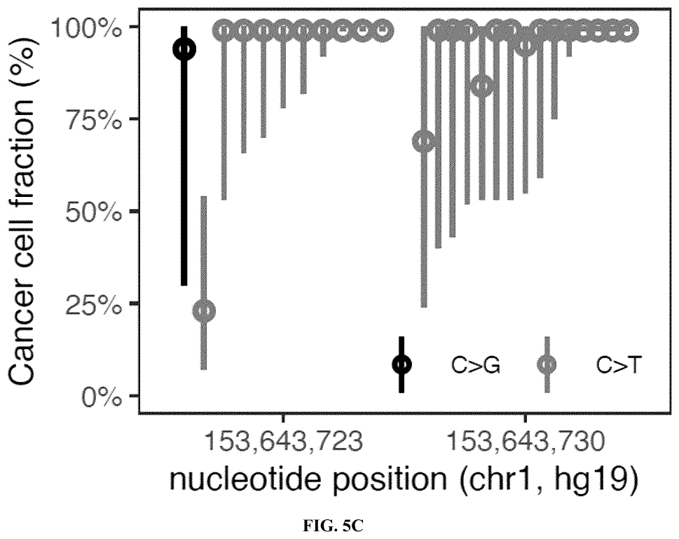

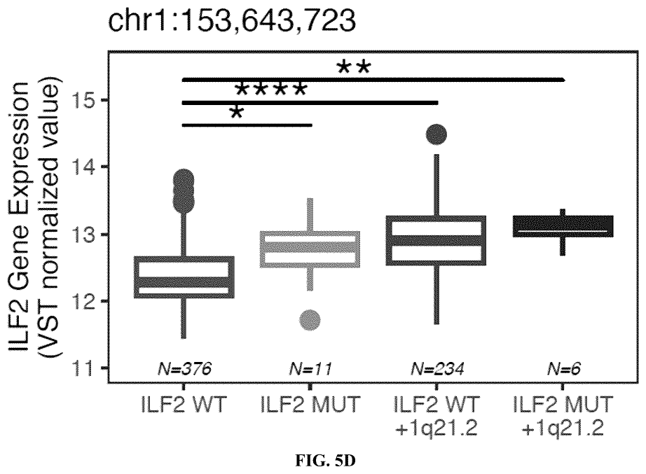

Because the algorithm to discover MM SV drivers focused on loss-of-function events, it did not capture well-known MYC events. MYC SVs are known to influence MM disease progression; however, they have been reported at various frequencies (0 to 35%). In the present Example, SVs were observed affecting MYC in 13 patients in the precursor setting for an estimated frequency of 8% (CI: 5 to 14%). Both Ig (IGH/IGL) (n=4) and non-Ig partner (n=9) genes were found, which is comparable with MM, and the most common non-Ig partner was BMP6/TXNDC5, also comparable with MM (FIG. 4D). However, in the MGUS population, MYC was notably rare (none in the PCROWD study, as compared with 1 case, progressor, from the Oben et al. study) (FIG. 5E). In SMM patients, Ig-MYC translocations also had a shorter TTP/adverse outcome/negative prognosis. Notably, when MYC-SVs occurred at the earliest stages of MGUS/SMM they were frequently cooccurring with RAS mutations (KRAS/NRAS) (and hyperdiploid) (FIGS. 1A-1 to 1A-5). The risk associated withMYC was captured by the MM-like score, which included focal gains and losses around 8q24 in some patients. Overall, this shows that genomic data from WGS can be used to discover new drivers and associate them with mutational processes that are likely to generate them and that are associated with risk and disease stage.

### Example 5: Non-Coding Candidate Drivers of Precursor Multiple Myeloma (MM)

Approximately 98% of the human genome is composed of “non-coding” elements, which include gene elements (3′ and 5′ untranslated regions (UTRs), introns, non-coding exons), regulatory elements (promoters, enhancers, insulators), and other elements (e.g., CpG islands not associated to a gene, transposable elements, etc.). Few non-coding elements such as TERT and FOXA1promoter regions have been shown to drive solid cancers; however, the existence and role of non-coding mutation hotspots in MM is largely undocumented. Here, DIG algorithm (Sherman, “Genome-wide mapping of somatic mutation rates uncovers drivers of cancer,” Nat Biotechnol, 40:1634-43 (2022)) was leveraged to discover non-coding MM drivers. Because the MM mutational landscape is likely to resemble that of normal B cell lineage (SHM, and off-target AID activity), the findings were compared with mutations from naïve and memory B cells obtained from Machado, et al. “Diverse mutational landscapes in human lymphocytes,” Nature, 608:742-32 (2022)(FIG. 5A). 66 candidate regions were found, of which 11 were found in immunoglobulin regions, 19 were in lncRNA/miRNA and enhancers, and 8 were possible technical artefacts after manual review.

Out of the 28 candidates, which were named by their gene's element (3′ UTR, 5′ UTR, Promoter), 18 were also found mutated in mature B cells and across all multiple myeloma (MM) subgroups (BCL6 promoter and 5′ UTR, BCL7A 5′UTR, BACH2 promoter and 5′ UTR, etc.; FIG. 5A, left). Mapping mutational signatures, it was confirmed that most were enriched in AID signatures SBS9, SBS85, consistent with off-target AID activity inherited from the B cell differentiation and were therefore unlikely to drive cancer. Out of the 10 remaining, 5 were found in known MM important genes or drivers (ILF2, BTG2, EGRI, CCND1 x2); 2 candidates overlapped with SV hotspots and were due to hypermutation around breakpoints (BMP6, TXNDC5), which were unlikely to drive cancer, and 3 were not investigated (STRIP1, DTX1, 25 SYBU).

All 10 candidates were mutated in subgroups of MM, which was consistent with known mutation mechanisms. For instance, CCND1 promoter and 5′ UTR, were hypermutated only in the CCND subgroup of patients and were marked by AID/SHM activity (FIG. 5A, bottom rows). This was consistent with off-target AID activity happening during and after the IgH-CCND1 translocation, defining the order of acquisition of initiating events. Conversely, the ILF2 promoter, on chromosome 1q21.2 was exclusively mutated in the MAF subgroup of patients (MAF, MAFA, MAFB translocations). ILF2 promoter was marked by APOBEC signatures, which were specific to MAF subgroups (FIG. 5A, top row). Specifically, APOBEC-induced TpC>T and TpC>G mutations were private to two nucleotide positions (FIG. 5B) which were not in hairpin loop but instead were marked by CTCF binding in ChIP-seq experiments in MM and in the GM12878 cell line. Those mutations were found across all disease stages and were confirmed in both the cohort and in the CoMMpass low-pass genomes. ILF2 promoter mutants were clonal in most cases (FIG. 5C), possibly facilitating their detection in low-pass genomes from CoMMpass. In CoMMpass with gene expression available and genomic coverage at the ILF2 promoter locus, ILF2-promoter mutants had higher expression of ILF2 as well as other genes within the same TAD, suggesting those mutations were selected for their effect beyond that of ILF2 expression (IL6R, S100A4, S100A6, TPM3 independent of 1q status; FIGS. 5D and 5E). Altogether, this provides the first exhaustive list of non-coding elements mutated in MM and its precursor conditions; with a novel non-coding candidate driver (ILF2-promoter), which associated with increased expression of known 1q targets; and a catalog of B-cell-specific off-target mutations marking the history of B to plasma cell differentiation.

The application of next-generation sequencing (NGS) in the multiple myeloma (MM) research setting over the past decade has highlighted marked intraclonal heterogeneity as a reservoir for tumor evolution and resistance. In the above Examples, analyses of the largest cohort of untreated precursor conditions is provided, providing a rich model of genomic landscape in MGUS and SMM demonstrating driver mutations and genetic heterogeneity being universal from the earliest stages and across disease stages. The new insights into precursor biology were possible due to the curation and analysis of 8× more cases spanning the precursor spectrum of MGUS and SMM—in addition to the use of deep unbiased whole genome analyses in selected tumor cells. Furthermore, a large collection of DNA sequencing data was harmonized, including whole genome and exome data, for the characterization and correlation of mutations and clinical outcomes using newly developed algorithms, resulting in a resource that serves as a reference map for the characterization of heterogeneity and genetic driver events present in multiple myeloma and its precursor conditions MGUS/SMM.

While extensive genetic characterization of MM samples has been carried out over the last decade, no mutations yet influence clinical decisions for patient care despite growing evidence of prognostic value in common MM genes. In the Examples provided herein, extensive genomic data was gathered from over 1,000 participants across the MM disease spectrum. A reference is provided for mutation frequencies across stages, which may be used to characterize asymptomatic to symptomatic conditions. In addition to subgroups enriched for asymptomatic patients and point mutations and copy-numbers more common in the overt disease setting, it was tested whether drivers more likely to be found in the MM setting would favor a shorter time to progression in MGUS/SMM. Indeed, it was shown that the “MM-like” score, which sums the number of mutants significantly enriched in MM patients, was associated with progression from SMM, an association that was validated in an independent cohort of SMM with longer follow-up.

A majority of MGUS/SMM cases were found to have an MM driver at the precursor stage. Thus, clinical targeted panel driver screening or WGS methods may be beneficial to understanding a patient's tumor biology and evolution. However, noting that BM biopsy is not standard of care for MGUS/SMM, conceivably it could be added to the routine, or alternatively, novel minimally invasive liquid biopsies that extract this information from circulating tumor cells (CTC)/cell free DNA (cfDNA) could be implemented.

This MM-like signature score not only associated with disease staging, risk classification, and time to progression from asymptomatic condition, but it also varied with time in untreated individuals. Noting patients with repeated longitudinal WGS within two years showed little to no evolution, while acquisition or emergence of MM drivers was exclusive to participants with 2 years waiting time in between samples, WGS may be repeated, and additionally provide insights on causes of progression and candidates drivers causing tumor growth or progression. Thus, this may represent an optimal time interval for bone marrow (BM) sampling and WGS as a method to capture patients evolving toward symptomatic disease. With patients being treated, rates of natural evolution may not hold in the treatment setting and require closer monitoring during periods of detectable residual disease, as well as times when no tumor WGS can be performed (undetectable residual disease). For MM specifically, it suggests that the mutational profile is shaped before the terminal PC differentiation takes place in the bone marrow.

Beyond gene-centric discovery and detection of MM drivers, WGS was used to estimate the absolute time of the last clonal sweep by leveraging the molecular clock which is assumed to accumulate clonal mutations at a constant rate. This approach provides an estimate of the earliest time of onset of the clonal disease in patients at diagnosis. Even though the approach necessitated clonal trisomies, which are less common in the MGUS setting, it was shown that MGUS and SMM, at the time of sampling in the clinic, may be relatively “young” tumors compared to MM. However, this was not the case for patients with SMM who later progressed to MM. SMM progressors, at biopsy, had tumors with comparable ages to overt MM, suggesting molecular timing was a proxy for estimating time before clinical progression. The data suggests that the first oncogenic events occur in the teens and early 20's of a person and that MGUS/SMM can be diagnosed early in the tumor's lifetime of progression, while MM can be diagnosed later in the lifetime of the tumor. Correlation between chronological age and tumor age may be helpful to identify the onset of MM in young individuals vs. those who are usually diagnosed at the age of mid-60's and 70's. Molecular timing leverages clonal trisomies; however, disease shows intratumor heterogeneity reflected by subclonal elements of the disease. Therefore, a clonality-based approach was developed to time events, which was based on the Bradley-Terry League competition model and run independently on MGUS/SMM and MM. The model showed a good correlation of clonality levels across disease stages, which was consistent with shared ontology and clonal history of the disease. Outliers in this model included genomic events that were subclonal in MGUS/SMM, but clonal in MM. Two events, gain(8q)(MYC) and del(12), were found, which were predicted to happen late in the history of MGUS/SMM, but early in MM, which may be interpreted as being events with strong competitive advantage for clonal growth. Indeed, MYC has been shown to be a highly important prognostic factor in MMs.

However, clinically this is usually tested by FISH, which does not have high sensitivity for detection compared to NGS-based methods. MYC SVs were detected in precursor MM cases; however, they were rare if not absent from MGUS disease. MYC had comparable translocation partners between SMM and MM, with separation of immunoglobulin and non-immunoglobulin partners (e.g., TXNDC5, TENT5C). This demonstrates both the utility and importance of transitioning to clinical next-generation sequencing NGS as an effective tool to capture the full resolution of clinically relevant tumor characteristics. Characterizing the full range of molecular events within a tumor may provide a better clinical understanding of the underlying biology and patient-specific progression risk.

Building on a new algorithm to extract candidate gene drivers hit by structural variants, it was confirmed that MGUS/SMM had less structural variation relative to MM except for initiating immunoglobulin translocation. As such, it was shown that immunoglobulin translocation correlated with a distinct mutational profile marked by somatic hypermutation marks, a physiological mechanism that generates B cell receptor sequences, and supported the in trans recombination of on-target effects of somatic hypermutation that causes cancer.

Finally, mutational processes and genomes from the normal B cell differentiation pathway were leveraged to classify non-coding cancer driver candidates into recurrent “off-target” somatic hypermutation versus APOBEC-induced hotspot candidate mutations. As such, the Examples presented herein provide the first reference for non-coding candidate driver elements in MM, which includes promoters and untranslated gene elements. The promoter of ILF2, which is found mutated with APOBEC TpC>[T/G] motif only in the MAF subgroup, and which gene expression correlates with mutation status in an external independent database, was identified as a solid non-coding driver across the MM spectrum.

To date, the application of next-generation sequencing (NGS) in multiple myeloma (MM) has mainly been at the research level with minimal translation to the clinical routine. Currently at the clinical level tumor biomarkers evaluation is carried out using molecular cytogenetics techniques, including FISH, which are based on analyses of a limited set of abnormality probes, which also vary across pathology departments. As whole-genome sequencing (WGS) is a genome-wide unbiased technology, it can also capture structural variants that shape the cancer genome and noncoding events that may affect phenotype and disease progression, which remains unexplored in precursor MM conditions. As such, given the access to comprehensive clinical data on patients studied in the above Examples, a head-to-head comparison of WGS vs FISH testing was performed to determine diagnostic yield. NGS performed better in the detection of actionable mutations informing risk stratification and intervention to prevent disease progression. Indeed, this was also shown in the acute myeloid leukemia (AML) setting with improved impact on detection and prognostics and has now been implemented as a routine clinical test, CHROMOseq. With a decade of genomic application in MM and well-cataloged mutations, it is also time to drive change in diagnostic pathology for MM and improve testing for patients to capture tumor biology early.

Analysis of the largest cohort of MGUS and SMM genomes to date was able to decipher the genomic history of MM, unraveling events including molecular clock signature, APOBEC, complex SVs, and MYC/RAS cooperation. This may further define a genomic risk model of disease progression independent of clinical tumor burden markers such as 2/20/20 in SMM. This highlights the potential of WGS to solve the “missing predictability” of current clinical models of progression.

### Example 6: MM-Like Score Correlated with Overall Survival (OS) and Progression-Free Survival (PFS) in Newly Diagnosed Multiple Myeloma

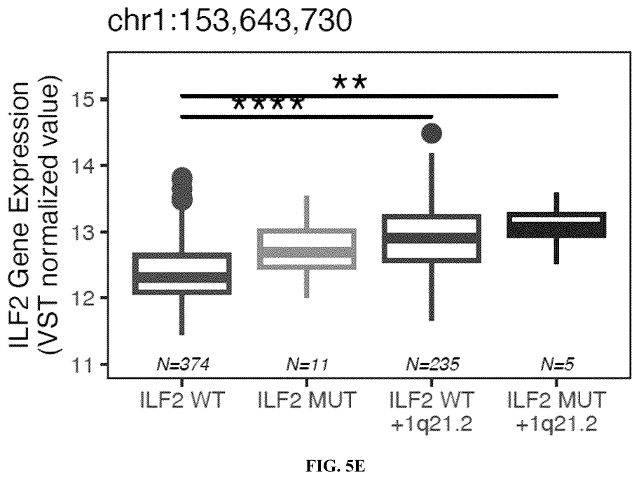

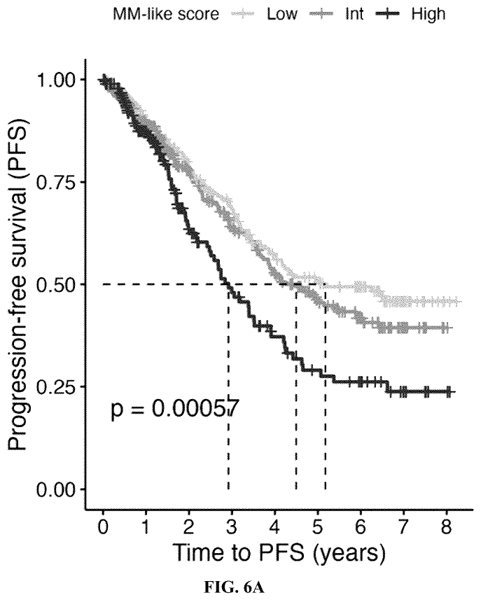

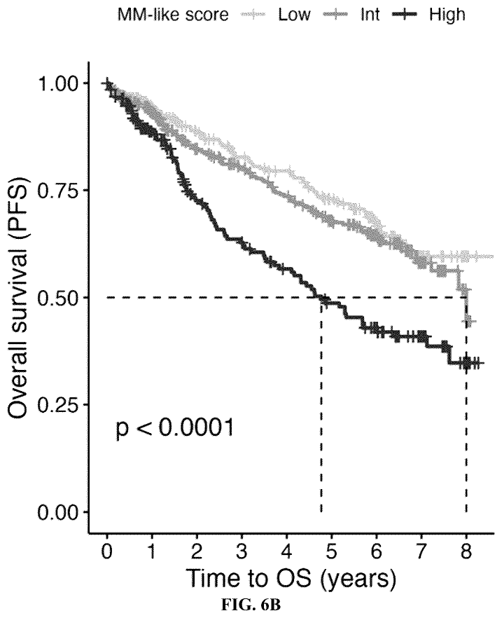

An analysis was undertaken to demonstrate that the MM-like score correlated with OS and PFS in newly diagnosed multiple myeloma patients (FIGS. 6A and 6B). The MM-like score distribution in the MMRF CoMMpass study (n=792) was split by quantiles (low/intermediate (int)/high) by MM-like score values calculated through analysis of the IA20 data release. In newly diagnosed MM patients, low, intermediate, and high scores were defined as follows: low: score of between 0 and 2; int: score of between 3 and 4; high: score of between 5 and 11.

What follows are a description of the calculations that were completed.

### PFS1:

Call:
coxph(formula=Surv(ttcpfsl, censpfsl) sig_class, data=mini.comm.surv.sig)
   coef exp(coef) se(coef) z p
sig_classInt 0.1503 1.1622 0.1366 1.100 0.27113
sig_classHigh 0.5684 1.7655 0.1534 3.706 0.00021
Likelihood ratio test=13.77 on 2 df, p=0.001024
n=792, number of events=300

### OS:

Call:
coxph(formula=Surv(ttcos, censos) sig_class, data=mini.comm.surv.sig)
   coef exp(coef) se(coef) z p
sig_classInt 0.1399 1.1502 0.1540 0.909 0.364
sig_classHigh 0.7159 2.0460 0.1605 4.461 8.15e-06
Likelihood ratio test=21.98 on 2 df, p=1.683e-05
n=792, number of events=259

The following methods were employed in the above examples.

### Calculation of Multiple Myeloma (MM)-Like Scores

MM-like scores were calculated by determining whether or not cancer cells of interest contained any of the markers listed in each row of Table 4 below. For each row of Table 4 containing one or more markers detected in the cancer cells of interest, the scores corresponding to each row were summed to determine the MM-like score. In Table 4, MA and MAFB are referenced because they are genes affected by the translocation.

TABLE 4
Summary of MM-like score calculation.

| | | Putative Drug | |
|---|---|---|---|
| | | Target for | |
| Score | Markers | Treatment | Comments |
| −1 | t(14;16)(MAF) | MAF | |
| −1 | t(14;20)(MAFB) | MAFB | |
| +1 | KRAS mutation (e.g., any | KRAS | |
| | substitution at position G12, | | |
| | G13, Q61, K117, and/or A146) | | |
| +1 | NRAS mutation (e.g., any | NRAS | |
| | substitution at position G12, | | |
| | G13, Q61, A146) | | |
| +1 | FAM46C mutation | FAM46C | FAM46C is also called |
| | | | TENT5C |
| +1 | Hyperdiploidy or gain(3q26.2) | GPR160 and | Hyperdiploidy is defined |
| | | TERC for | as the clonal gain of two |
| | | gain(3q26.2) | or more odd chromosomes |
| | | | among chr3, 5, 7, 9, 11, |
| | | | 15, 17, 19, and 21. |
| +1 | Del(1p) or del(1p22.1) or | FAM46C | 1p22.1, 1p12, 1p32 are |
| | del(1p12) or del(1p32) | (1p12), EVI5 | often redundant and are all |
| | | (1p22.1), | part of 1p |
| | | CDKN2C | |
| | | (1p32.3) | |
| +1 | Gain(1q) or gain(1q21.2) | MCL1, CKS1B, | 1q21.2 and 1q are often |
| | | BCL9 | found together, and |
| | | | 1q21.2 is part of 1q |
| +1 | Del(4p16.3) | FGFR3 and | NSD2 is also called |
| | | NSD2 | MMSET or WHSC1 |
| +1 | Del(4q34.3) | CASP3 | CASP3 is a new potential |
| | | | target in MM |
| +1 | Del(8p) or del(8p23.3) | MCPH1 | MCPH1 is a new potential |
| | | | target in MM |
| +1 | Del(8q24.21) or gain(8q24.21) | MYC | Structural variant of the |
| | or structural variation of MYC | | MYC locus is the most |
| | | | general biomarker, |
| | | | however, is hard to detect. |
| | | | A surrogate, or secondary |
| | | | characterization, is local |
| | | | copy number gain or |
| | | | deletion around 8q24.21. |
| | | | In addition, FISH can |
| | | | recognize “split of MYC |
| | | | probe” which would be |
| | | | considered a MYC |
| | | | structural variant. |
| +1 | Del(16q) or del(16q12.1) | CYLD | |
| +1 | Del(17q21.2) | IKZF3 | IKZF3 is a new potential |
| | | | target in MM (it is a |
| | | | known driver of CLL and |
| | | | DLBCL, two other |
| | | | cancers) |

### Patient Sample Collection and Plasma Cell Enrichment

Bone marrow (BM) samples were collected from the Precursor Crowd (PCROWD) study at the Dana-Farber Cancer Institute (MR #14-174). BM samples were drawn into heparinized EDTA tubes before mononuclear cell isolation (SepMate, STEMCELL Technologies) and subsequent plasma cells (PC) selection using CD138 magnetic-activated cell sorting (MACS) (Miltenyi Biotec). In patients with low tumor burden, a standardized CellSearch platform was used for tumor cell enrichment (Dutta, et al., “MinimuMMIN/-seq: Genome Sequencing of Circulating Tumor Cells for Minimally Invasive Molecular Characterization of Multiple Myeloma Pathology,” Cancer Discov. 13:348-63 (2023), doi: 10.1158/2159-8290.CD-22-0482). Enriched PC counts and viability of MACS-sorted BM samples were assessed by trypan blue exclusion on an automated cell counter (Invitrogen Countess 3, Thermo Fisher Scientific). Each patient also had peripheral blood processed for isolation of peripheral blood mononuclear cells (PBMCs). Samples were cryopreserved for subsequent downstream molecular analyses. Patients with BM biopsies had routine molecular cytogenetics and FISH studies performed at clinical pathology laboratories.

### DNA Isolation and Library Construction

Genomic DNA isolation was carried out using the Monarch Genomic DNA Purification Kit (New England Biolabs), with tumor (PCs) and normal (PBMCs) yields quantified by Qubit 3.0 fluorometer (Thermo Fisher Scientific). 50 ng-100 ng was taken forward for DNA sequencing library preparation using the NEBNext Ultra II FS DNA Library Prep kit (New England Biolabs) with unique dual index adapters (NEBNext Multiplex Oligos) according to manufacturers' instructions. Final library fragment sizes were assessed using the BioAnalyzer 2100 (Agilent Technologies), and yields quantified by Qubit 3.0 fluorometer (Thermo Fisher Scientific) and qPCR (KAPA Library Quantification Kit).

### Whole Genome Sequencing (WGS) and Analyses

Final sample libraries were normalized and pooled for matched tumor/normal whole genome sequencing (WGS). WGS was performed on Illumina Novaseq6000 instrument, with loading on S4 flowcells, 2×150 bp paired-end reads. Data was aligned to GRCh38 reference genome with bwa (Li 2013 BioRxiv), duplicates were marked with Picard MarkDuplicates, and base score recalibration was performed with GATK BQSR. Mutations were detected with MuTect for single nucleotide variants (SNV), and with Strelka2 for small insertions and deletions (indels). Candidate mutants were filtered through a Panel of Normals consisting of PBMCs processed with the same library preparation protocol, for orientation bias inducing artifact (oxoG, FFPE), for mapping artifacts with the BLAT realignment, and for enzymatic shearing cruciform variant artifacts. deTiN was used to estimate tumor-in-normal (TiN) contamination rate and rescue candidate mutations accordingly under 10% TiN. Fingerprinting was used to confirm matching tumor-normal pairing (GATK CrosscheckFingerprints log of the odds metrics), ConEst was used to detect exogenous contamination, and BovoBlat to exclude two samples with bovine DNA contamination likely from bovine fetal serum. Finally, candidate mutations were annotated with GATK Funcotator. Allelic copy number ratios were calculated with the AllelicCapSeg method against this study's Panel of Normals, and ABSOLUTE was used to estimate purity, ploidy, and cancer cell fraction of mutations. Subclonal structures were deciphered with the PhylogicNDT suite of tools. Structural variants (SVs) were detected with Manta, SvABA, and dRanger/BreakPointer. Consensus lists of candidate SVs were then established and manually reviewed on IGV.

### Discovery of Driver Candidates and Mutational Processes

Mutational processes were quantified with the hdp R package and fitted against a list of known mutational processes at stake in hematologic malignancies allowing for hybrid capture of known and novel signatures. Coding drivers were discovered with the MutSig2CV method. Copy-number hotspots and associated genes were discovered with the GISTIC2.0 method. Non-coding hotspots were discovered with the DIG method and filtered for candidate hotspots found with the same method on WGS from normal lymphocytes.

### Mutational Signature Extraction and Deconvolution

Mutational signatures were extracted from SNVs across all donors using a Bayesian hierarchical Dirichlet process (hdp package) (github.com/nicolaroberts/hdp). If a given donor had multiple different tumor samples sequenced, SNVs were grouped into pseudo-samples based on their presence or absence across the different samples, such that a given SNV was only counted once. SNVs were further divided into three categories, each forming a further pseudo-sample: SNVs within immunoglobulin genes, clustered SNVs with an inter-mutational distance less than 1,000 bases, and other SNVs. SNVs were collapsed into their 96 counts of base change within their trinucleotide context. The hierarchy used by hdp consisted of (pseudo-)samples grouped by donor. The hdp algorithm was run in 20 different chains, for 40,000 iterations with a burn-in of 20,000 iterations.

The resulting extracted signatures were further compared to the reference COSMIC 25 signatures (v3.3). Each signature extracted by hdp was modelled as a linear combination of reference signatures previously described in lymphoid cancers or normal lymphocytes using an expectation maximization algorithm, as previously described (Moore, et al., Nature, 597:381-386, 2021). If the cosine similarity of the extracted signature and reconstructed signature exceed 0.85, the extracted hdp signature was replaced with its constituent COSMIC reference signature. If not, the hdp signature was kept as a new mutational signature.

### Timing of Hyperdiploidy

The acquisition of (sub-)chromosomal duplications can be timed using the relative ratio of duplicated and non-duplicated SNVs (Gerstung, et al. Nature, 578:122-128 (2020)). In brief, this ratio can be estimated using a binomial mixture model (Coorens, et al. New England Journal of Medicine, 383:1860-1865 (2020)) on the counts of variant supporting reads and total depth on the chromosomal regions affected by duplications, taking into account the purity of the tumor and the local copy number profile. From these ratios, the relative timing can be calculated:

$$T = \frac{{C}_{M} + {C}_{m}}{{C}_{M} + \frac{{P}_{ND}}{{P}_{D}}}$$

Where C_{M} and Cₘ are the major and minor copy number, respectively, and P_{D} and P_{ND} the proportion of mutations assigned to the duplicated or non-duplicated copy number. The timepoint of duplication will be a number between 0 and 1, the former representing the zygote and the latter the most recent common ancestor to the clone, i.e., MGUS, SMM or MM. For purposes of practicality, the 1 is often taken to represent the time of sampling, which will give an upper bound of the timing of the hyperdiploidy.

This approach assumes a constant mutation rate before and after duplications. Therefore, only SNVs that were most likely due to SBS1 and SBS5 (both clock-like signatures) were used for the timing analysis.

### Timing of Acquisition of Events

Timing of events was performed with two complementary approaches: first, a novel method termed “Clonality League Comparison” (CLC) (github) was developed that performed a Bradley-Terry league model analysis of total copy-number ratios, allowing integration of exome and genome data generated over more than a decade including at lower depth of sequencing and/or lower purity. CLC was run independently on MGUS/SMM and MM cohorts, and Bradley-Terry scores for each copy-number driver were compared with and across runs. Second, for genomes with deep coverage (excluding MMRF CoMMpass cohort), a binomial mixture model was fitted on each patient's clonally gained hyperdiploidy chromosome (3, 5, 7, 9, 11, 15, 19, 21) to estimate the ratio of duplicated versus non-duplicated clonal mutations. The relative age of the last clonal sweep obtained from the mixture model was then linearly scaled to an absolute scale with the age of patients at biopsy.

### Cell-of-Origin Prediction Analysis

A previously described methodology was used with some modifications (Kübler, et al., bioRxiv. 2019:517565. doi: 10.1101/517565; and Machado, et al. Nature, 608:742-32 (2022)). Briefly, aggregated genome-wide somatic tumor mutation density was modeled from ChIP-sequencing reads of normal B cell types using random forest regression with 10-fold cross-validation. For each tumor mutational profile, separate models were trained using all ChIP-sequencing profiles associated with each B cell type, combining the two replicates. Clonal and subclonal tumor mutations were counted in 2,125 1 Mb genomic windows, and, in addition to the previously reported excluded regions, the windows spanning the heavy (IGH) and light (Kappa and Lambda) chains were also excluded. B cell type epigenetic data with replicates were collected from IIHEC and EnCODE (ERS663722, ERS663732, ERS643304, ERS728717, ERS663728, ERS728718, CEMT0032.gDNA, ENCDO412QUR, CEMT0135.gDNA, CEMT0134.gDNA) using the following ChIP-sequencing data: Control, H3K27ac, H3K27me3, H3K36me3, H3K4mel, H3K4me3, H3K9me3. Prediction analysis was made using Julia 1.6.1 in a Jupyter Notebook environment. Statistical significance was determined between each top hit and the subsequent models using a two-tailed corrected t-test on the values from the 10-fold cross-validation, taking into account the interdependence of tests in a cross-validation setting.

### Clonality-Based League Competition

To account for differences in sample preparation and sequencing methods, the CLC method leveraged both total copy number ratio estimates of chromosome arms and focal alterations (significant by GISTIC2). CLC then compared clonality estimates of copy-number events with a Bradley-Terry model to determine a precise ranking of copy-number subclonality.

### Genomic Data Collection

Next-generation sequencing data from previous multiple myeloma (MM) studies were analyzed with the same methods for comparative analyses, including WGS of the MMRF CoMMpass database (dbGAP Accession number phs000748.v1.p1, n=792), such as phs003084.v1, EGAD00001006363, and EGAS00001004467.

### Statistical Analyses

Quantitative variables were described with their median and interquartile range unless stated otherwise. The significance of the average difference between groups was tested with the Kruskal-Wallis test for multiple group testing followed by Dunn posthoc tests. When only two groups were compared, the Wilcoxon test was used. Means estimated from random processes are given with their 95% confidence interval unless stated otherwise. Qualitative variables were described using the frequency of their respective modalities. Distinct distribution between groups was assessed with χ² Pearson test (or Fisher exact test where appropriate). In survival analyses, patients were stratified per the median of quantitative variables, and time-to-event was calculated from the date of sampling to clinical progression to MM or death of any cause, whichever happened first. Patients were censored at the date of last clinical follow-up, or at the start of treatment for MGUS/SMM where appropriate. Significance of Cox progression models was assessed with the Log-rank test. P values were adjusted with the fdr control procedure and were considered significant below 0.05.

### OTHER EMBODIMENTS

From the foregoing description, it will be apparent that variations and modifications may be made to the embodiments and aspects of the disclosure described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-20. (canceled)

21. A method for determining progression of a subject to symptomatic cancer from an asymptomatic state, comprising:
(a) obtaining a biological sample of said subject with said asymptomatic state, wherein said biological sample comprises one or more plasma tumor cells;
(b) determining a multiple myeloma (MM)-like score for said subject, based on analyzing sequenced genomic data of said one or more plasma tumor cells and assigning a plurality of values to mutations of one or more marker genes of said one or more plasma tumor cells identified from said sequenced genomic data; and
(c) determining a progression of said subject to said symptomatic cancer from said asymptomatic state, based at least in part on said MM-like score indicating said symptomatic cancer.

22. The method of claim 21, comprising performing whole genome amplification of one or more nucleic acid molecules of said one or more plasma tumor cells thereby generating an amplified one or more nucleic acid molecules.

23. The method of claim 22, comprising performing whole genome sequencing of said amplified one or more nucleic acid molecules of said one or more plasma tumor cells to determine said mutations of said one or more marker genes of said one or more plasma tumor cells.

24. The method of claim 23, wherein said whole genome sequencing comprises enriching said one or more plasma tumor cells from said biological sample and sequencing polynucleotides from said enriched one or more plasma tumor cells.

25. The method of claim 24, wherein said whole genome sequencing comprises sequencing said polynucleotides to a coverage of at least about 10-fold.

26. The method of claim 21, comprising administering a treatment to treat said symptomatic cancer of said subject.

27. The method of claim 26, wherein said treatment comprises a chemotherapeutic agent, radiation, immunotherapy, or any combination thereof.

28. The method of claim 21, wherein said mutations of said one or more marker genes comprise hyperdiploidy, wherein said hyperdiploidy comprises a clonal gain of two or more odd chromosomes selected from chromosomes 3, 5, 7, 9, 11, 15, 17, 19, and 21.

29. The method of claim 21, wherein said mutations of said one or more marker genes comprise one or more copy number alterations comprising gain(1q), gain(1q21.2), del(1p), del(1p22.1), del(1p12), del(1p32), del(4p16.3), del(4q34.3), del(8p), del(8p23.3), del(8q24.21), gain(8q24.21), del(16q), del(16q12.1), del(17q21.2), or any combination thereof.

30. The method of claim 21, wherein said MM-like score indicating said symptomatic cancer is an MM-like score of greater than or equal to 4.

31. The method of claim 21, wherein said plurality of values assigned to mutations comprises assigning a value of +1 for each of one or more mutations enriched in symptomatic multiple myeloma relative to said asymptomatic state, and assigning a value of −1 for each of one or more mutations enriched in said asymptomatic state relative to symptomatic multiple myeloma.

32. The method of claim 21, wherein said one or more marker genes comprise one or more of KRAS, NRAS, FAM46C, MAF, and MAFB.

33. The method of claim 21, wherein said biological sample is a blood sample or a bone marrow sample.

34. The method of claim 21, wherein said method is performed on a plurality of biological samples obtained from said subject at different time points, and wherein said determining said progression comprises monitoring an increase in said MM-like score over time as indicative of progression toward said symptomatic cancer.

35. The method of claim 34, wherein said plurality of biological samples are obtained at intervals of at least about 24 months, and wherein an increase in said MM-like score between time points is driven by acquisition of one or more additional driver mutations selected from the group consisting of KRAS mutations, NRAS mutations, del(1p), and hyperdiploidy.

36. The method of claim 21, further comprising estimating a molecular age of said one or more plasma tumor cells based on a ratio of duplicated to non-duplicated clock-like somatic mutations in one or more clonally gained chromosomes of said one or more plasma tumor cells, wherein said molecular age of said one or more plasma tumor cells is indicative of a likelihood of progression to said symptomatic cancer.

37. The method of claim 36, wherein said clock-like somatic mutations are attributable to mutational signatures SBS1 and SBS5, and wherein said estimated molecular tumor age is calculated by multiplying a fraction of time from a zygote to acquisition of a clonal hyperdiploidy event by said subject's age at a time of obtaining said biological sample from said subject.

38. The method of claim 21, wherein said MM-like score is validated as independently associated with progression from SMM to symptomatic multiple myeloma with a hazard ratio of at least about 2.8 in a multivariate regression model.

39. The method of claim 21, wherein said one or more plasma tumor cells are enriched from said biological sample by CD138 magnetic-activated cell sorting (MACS) prior to said determining step.

40. The method of claim 21, wherein said mutations of said one or more marker genes further comprise a structural variation of MYC, wherein said structural variation of MYC comprises a translocation of chromosome 8q24 with an immunoglobulin locus partner or a non-immunoglobulin partner, and wherein said structural variation of MYC is detected by whole genome sequencing or fluorescence in situ hybridization (FISH).
